(19) United States
(12) Patent Application Publication
(10) Pub. No.: US 2023/0312698 A1
(43) Pub. Date: Oct. 5, 2023

(54) ANTIBODIES AGAINST HUMAN ANGIOPOIETIN-2 AND USES THEREOF

(71) Applicant: Nanjing GenScript Biotech Co., Ltd., Nanjing (CN)

(72) Inventors: Yi Jin, Nanjing (CN); Zhongdao Li, Nanjing (CN); Wenshuang Jia, Nanjing (CN); Brian Hosung Min, Nanjing (CN); Weiming Wang, Nanjing (CN)

(73) Assignee: Nanjing GenScript Biotech Co., Ltd., Nanjing (CN)

(21) Appl. No.: 18/040,615

(22) PCT Filed: Aug. 6, 2021
(86) PCT No.: PCT/CN2021/111109

§ 371 Date: Feb. 3, 2023

(30) Foreign Application Priority Data
Aug. 7, 2020 (WO) PCT/CN2020/107798
Feb. 20, 2021 (WO) PCT/CN2021/077108

(51) Int. Cl. C07K 16/22; C12N 15/63; A61P 43/00

(52) U.S. Cl. CPC C07K 16/22; C12N 15/63; A61P 43/00; C07K 2317/565; C07K 2317/71; C07K 2317/24; C07K 2317/31

(57) ABSTRACT

The invention relates to an antibody or an antigen-binding portion thereof that specifically binds to Ang2, blocking Ang2 binding to Tie2 or integrins, or triggering Ang2 clusting and Tie2 signaling activation.

### CROSS REFERENCE TO RELATED APPLICATIONS

This is the U.S. National Stage of International Application No. PCT/CN2021/111109, filed Aug. 6, 2021, which was published in English under PCT Article 21(2), which in turn claims the benefit of International Application No. PCT/CN2020/107798, filed Aug. 7, 2020, and International Application No. PCT/CN2021/077108, filed Feb. 20, 2021. International Application No. PCT/CN2021/111109 is incorporated herein by reference in its entirety.

### INCORPORATION BY REFERENCE

All documents cited or referenced herein (including without limitation all literature documents, patents, published patent applications cited herein) (“herein cited documents”), and all documents cited or referenced in herein cited documents, together with any manufacturer's instructions, descriptions, product specifications, and product sheets for any products mentioned herein or in any document incorporated by reference herein, are hereby incorporated herein by reference, and may be employed in the practice of the invention. More specifically, all referenced documents are incorporated by reference to the same extent as if each individual document was specifically and individually indicated to be incorporated by reference. Any Genbank sequences mentioned in this disclosure are incorporated by reference with the Genbank sequence to be that of the earliest effective filing date of this disclosure.

Citation or identification of any document in this application is not an admission that such document is available as prior art to the present invention.

### FIELD OF THE INVENTION

The present disclosure relates to antibodies or antigen-binding portions thereof that specifically bind to angiopoietin-2 (Ang2) or a fragment thereof, and their uses for diagnosing or treating Ang2 related cancers, inflammatory diseases and infectious diseases.

### INCORPORATION OF ELECTRONIC SEQUENCE LISTING

The electronic sequence listing, submitted herewith as a TXT file named Sequence Listing.txt (234,360 bytes), created on Feb. 2, 2023, is herein incorporated by reference in its entirety.

### BACKGROUND OF THE INVENTION

Angiogenesis occurs throughout life and is necessary for e.g., embryologic development and tissue repair. It is a complex process that is driven by extracellular matrix-derived angiogenic inhibitors and/or growth factors including vascular endothelial growth factor (VEGF), fibroblast growth factor (FGF), insulin-like growth factor (IGF), and angiopoietins. The imbalance of the pro- and anti-angiogenic signaling may lead to abnormal vascular network characterized by hyperpermeable vessels, as observed in e.g., cancers, sepsis, and neovascular age-related macular degeneration, leading to endothelial, immune and/or lesion cell migration, deteriorated drug delivery, and the like (Akwii R G et al., (2019) Cells 8(5):471). The underlying molecular mechanisms behind the pathological capillary leakage is under intensive studies.

VEGF is the most widely exploited target for antiangiogenic therapy. The anti-VEGF therapy has been reported to enable “vascular normalization”, bringing vasculature towards a more “normal” phenotype with reduced hyperpermeability (Goel S et al., (2011) Physiological Reviews 91(3):1071-1121). However, not all patients respond to such treatments. For example, for the treatment of retinal vascular diseases, the anti-VEGF medication is effective in a significant number of patients, but cannot prevent the progression to legal blindness in many other patients.

Recently, increased attention has been directed toward the Tie2 pathway. Tie2 is an endothelial cell-specific receptor tyrosine kinase (RTK), and its activation plays a central role in vascular stability. Studies have shown Tie2 is modulated by two secreted proteins, angiopoietin-1 (ANGPT-1, Ang1) and angiopoietin-2 (ANGPT-2, Ang2). Ang1 acts as a Tie2 agonist, phosphorylating Tie2 and leading to activation of the p85 subunit of PI3K and Akt phosphorylation on Ser473. The Ang1-Tie2 interaction induces various protective downstream pathways, including stabilization of vascular endothelial cells and attenuation of vascular permeability. Ang2 was initially reported as an endogenous Tie2 antagonist by competing with Ang1 over Tie2 binding. Recent data demonstrates that Ang2 has context-dependent Tie2 agonistic activities, where it induces Tie2 phosphorylation, resulting in activation of the p85 subunit of PI3K and Akt phosphorylation on Ser473. Ang2 also binds to integrins in a Tie2 independent manner, destabilizing the endothelium and inducing endothelial cell migration (Felcht M et al., (2012) Journal of Clinical Investigation 122(6):1991-2005; Hakanpaa L et al., (2015) Nature Communications 6: 5962). The co-presence of Ang2 and VEGF may even provide an additive effect on vascular permeability.

Ang2 levels are low under normal physiological conditions, but up-regulated during inflammation or cancer. For example, high Ang2 levels have been found in the serum or lesion tissues of subjects with autoimmune diseases, pneumonia, mycoplasma pulmonis infection, sepsis, certain cancers, cardiovascular diseases, and diabetic retinopathy (Akwii R G et al., (2019) supra). Ang2 acts on endothelial cells and pericytes, causing e.g., pericyte detachment from the basement membrane, and immune and/or cancer cell migration (Geranmayeh M H et al., (2019) Cell Communication and Signaling 17: 26).

Ang2 blockade has been found effective in alleviating several conditions in pre-clinical studies, including mycoplasma pulmonis infection, sepsis, lung cancers, cervical cancers, and diabetic retinopathy (Tabruyn S P et al., (2010) The American Journal of Pathology 177(6): 3233-3243; Leligdowicz A et al., (2018) Front Immunol. 9:838; Oliner J et al., (2004) Cancer Cell 6(5): 507-516; Papadopoulos K P et al., (2015) Clinical Cancer Research 22(6): 1348-1355; Yang P et al., (2017) Tumour Biol. 39(7): 1010428317711658; Yun J H et al., (2016) Cell Death Dis. 7(2): e2101), and are currently tested under clinical trials. Trebananib, a fusion protein that blocks both Ang1 and Ang2 binding to Tie2, prolonged the progression-free survival of certain patients having recurrent ovarian cancer in phase 3 trial (Monk B J et al., (2014) Lancet Oncol. 15(8): 799-808). However, MEDI3617, a selective Ang2 inhibitor, showed limited efficacy in patients having ovarian cancer or glioma. Therefore, there is a need for more Ang2 inhibitors such as anti-Ang2 antibodies with better therapeutic characteristics.

Further, in the context of several conditions like sepsis, Ang2 expression is increased, while Ang1 and Tie2 expressions decline. Ang2 targeting therapeutics that not only block Ang2 binding to Tie2 but also have Tie2 agonistic activities may be preferred, as the Tie2 signaling activation is responsible for vascular normalization. ABTAA, an anti-Ang2 antibody that binds Ang2 and activates Tie2 signaling, showed better anti-tumor effects in an animal study, as compared to an anti-Ang2 antibody lack of Tie2 activation activity (Park J S. et al., (2016) Cancer Cell. 30(6):953-967). This antibody was also tested in a neovascular age-related macular degeneration mouse model where it suppressed choroidal neovascularization and vascular leakage, and regenerated the choriocapillaris (Kim J et al., (2019) Science Advances 5(2): eaau6732).

### SUMMARY OF THE INVENTION

The present disclosure provides an isolated monoclonal antibody or an antigen-binding portion thereof, that specifically binds to Ang2 or a fragment thereof, (i) inhibiting Ang2 binding to Tie2 or integrins, or (ii) directly activating Tie2 signaling, such that the antibody or antigen-binding portion thereof may inhibit unrequired angiogenesis and/or restore vascular stability to some extent.

Without wishing to be bound by the theory, the anti-Ang2 antibody or antigen-binding portion thereof of the disclosure may activate Tie2 signaling by (i) blocking Ang2 binding to Tie2 such that Ang1 binds and phosphorates Tie2; or (ii) binding to Ang2 and triggering Ang2 clustering and subsequent Tie2 activation. Without wishing to be bound by the theory, in addition to Tie2 activation, the anti-Ang2 antibody or antigen-binding portion thereof of the disclosure may stabilize the endothelium by blocking Ang2 binding to integrins.

The antibody or the antigen-binding portion thereof may be used in detection of Ang2 proteins in vitro, and diagnosis or treatment of Ang2 related diseases.

Accordingly, in one aspect, the present disclosure pertains to an isolated monoclonal antibody, or an antigen-binding portion thereof, that binds to Ang2 or a fragment thereof, having (a) a heavy chain variable region which may comprise a VH CDR1, a VH CDR2 and a VH CDR3 comprising the amino acid sequences of (1) SEQ ID NOs: 1, 23 and 60, respectively; (2) SEQ ID NOs: 2, 24 and 61, respectively; (3) SEQ ID NOs: 3, 25 and 62, respectively; (4) SEQ ID NOs: 3, 26 and 62, respectively; (5) SEQ ID NOs: 4, 27 and 63, respectively; (6) SEQ ID NOs: 5, 28 and 64, respectively; (7) SEQ ID NOs: 6, 29 and 65, respectively; (8) SEQ ID NOs: 1, 25 and 62, respectively; (9) SEQ ID NOs: 7, 30 and 66, respectively; (10) SEQ ID NOs: 8, 29 and 65, respectively; (11) SEQ ID NOs: 4, 31 and 63, respectively; (12) SEQ ID NOs: 5, 32 and 67, respectively; (13) SEQ ID NOs: 3, 33 and 68, respectively; (14) SEQ ID NOs: 9, 34 and 69, respectively; (15) SEQ ID NOs: 1, 35 and 62, respectively; (16) SEQ ID NOs: 4, 27 and 70, respectively; (17) SEQ ID NOs: 4, 27 and 71, respectively; (18) SEQ ID NOs: 10, 36 and 72, respectively; (19) SEQ ID NOs: 4, 37 and 73, respectively; (20) SEQ ID NOs: 11, 38 and 74, respectively; (21) SEQ ID NOs: 12, 39 and 75, respectively; (22) SEQ ID NOs: 13, 40 and 76, respectively; (23) SEQ ID NOs: 14, 41 and 77, respectively; (24) SEQ ID NOs: 5, 42 and 64, respectively; (25) SEQ ID NOs: 5, 43 and 78, respectively; (26) SEQ ID NOs: 15, 44 and 79, respectively; (27) SEQ ID NOs: 8, 45 and 80, respectively; (28) SEQ ID NOs: 16, 46 and 81, respectively; (29) SEQ ID NOs: 2, 47 and 61, respectively; (30) SEQ ID NOs: 17, 48 and 82, respectively; (31) SEQ ID NOs: 11, 38 and 83, respectively; (32) SEQ ID NOs: 10, 49 and 84, respectively; (33) SEQ ID NOs: 2, 50 and 61, respectively; (34) SEQ ID NOs: 10, 36 and 85, respectively; (35) SEQ ID NOs: 5, 32 and 86, respectively; (36) SEQ ID NOs: 18, 51 and 87, respectively; (37) SEQ ID NOs: 2, 52 and 88, respectively; (38) SEQ ID NOs: 19, 53 and 89, respectively; (39) SEQ ID NOs: 5, 32 and 64, respectively; (40) SEQ ID NOs: 20, 54 and 90, respectively; (41) SEQ ID NOs: 21, 55 and 91, respectively; (42) SEQ ID NOs: 21, 56 and 91, respectively; (43) SEQ ID NOs: 21, 55 and 346, respectively; (44) SEQ ID NOs: 22, 57 and 347, respectively; (45) SEQ ID NOs: 21, 58 and 91, respectively; (46) SEQ ID NOs: 21, 59 and 91, respectively; or (47) SEQ ID NOs: 249, 250 and 251, respectively; and/or (b) a light chain variable region which might comprise a VL CDR1, a VL CDR2 and a VL CDR3 comprising the amino acid sequences of (1) SEQ ID NOs: 92, 114 and 133, respectively; (2) SEQ ID NOs: 93, 115 and 134, respectively; (3) SEQ ID NOs: 92, 116 and 135, respectively; (4) SEQ ID NOs: 92, 116 and 136, respectively; (5) SEQ ID NOs: 92, 117 and 137, respectively; (6) SEQ ID NOs: 94, 118 and 138, respectively; (7) SEQ ID NOs: 95, 119 and 139, respectively; (8) SEQ ID NOs: 96, 116 and 136, respectively; (9) SEQ ID NOs: 97, 120 and 140, respectively; (10) SEQ ID NOs: 98, 121 and 141, respectively; (11) SEQ ID NOs: 99, 122 and 142, respectively; (12) SEQ ID NOs: 100, 123 and 143, respectively; (13) SEQ ID NOs: 101, 124 and 144, respectively; (14) SEQ ID NOs: 97, 120 and 145, respectively; (15) SEQ ID NOs: 102, 115 and 146, respectively; (16) SEQ ID NOs: 103, 115 and 147, respectively; (17) SEQ ID NOs: 104, 125 and 148, respectively; (18) SEQ ID NOs: 93, 115 and 149, respectively; (19) SEQ ID NOs: 105, 126 and 150, respectively; (20) SEQ ID NOs: 106, 116 and 142, respectively; (21) SEQ ID NOs: 97, 120 and 151, respectively; (22) SEQ ID NOs: 97, 120 and 152, respectively; (23) SEQ ID NOs: 103, 115 and 149, respectively; (24) SEQ ID NOs: 107, 127 and 153, respectively; (25) SEQ ID NOs: 108, 128 and 154, respectively; (26) SEQ ID NOs: 109, 116 and 136, respectively; (27) SEQ ID NOs: 110, 129 and 155, respectively; (28) SEQ ID NOs: 111, 130 and 156, respectively; (29) SEQ ID NOs: 112, 131 and 157, respectively; (30) SEQ ID NOs: 111, 132 and 158, respectively; (31) SEQ ID NOs: 111, 130 and 159, respectively; (32) SEQ ID NOs: 113, 115 and 134, respectively; or (33) SEQ ID NOs: 252, 253 and 254, respectively. Also provided are variants of these antibodies or antigen-binding portions that comprise up to about 3 amino acid substitutions (e.g., one, two, or three amino acid substitutions) in each of the CDRs.

The isolated monoclonal antibody, or the antigen-binding portion thereof, of the present disclosure may comprise a VH CDR1, a VH CDR2, a VH CDR3, a VL CDR1, a VL CDR2, and a VL CDR3 which may comprise the amino acid sequences of (1) SEQ ID NOs: 1, 23, 60, 92, 114 and 133, respectively; (2) SEQ ID NOs: 2, 24, 61, 93, 115 and 134, respectively; (3) SEQ ID NOs: 3, 25, 62, 92, 114 and 133, respectively; (4) SEQ ID NOs: 3, 26, 62, 92, 114 and 133, respectively; (5) SEQ ID NOs: 4, 27, 63, 92, 116 and 135, respectively; (6) SEQ ID NOs: 5, 28, 64, 92, 114 and 133, respectively; (7) SEQ ID NOs: 6, 29, 65, 92, 116 and 136, respectively; (8) SEQ ID NOs: 1, 25, 62, 92, 114 and 133, respectively; (9) SEQ ID NOs: 7, 30, 66, 92, 117 and 137, respectively; (10) SEQ ID NOs: 8, 29, 65, 92, 116 and 136, respectively; (11) SEQ ID NOs: 4, 31, 63, 92, 116 and 135, respectively; (12) SEQ ID NOs: 5, 32, 67, 92, 114 and 133, respectively; (13) SEQ ID NOs: 3, 33, 68, 92, 114 and 133, respectively; (14) SEQ ID NOs: 9, 34, 69, 94, 118 and 138, respectively; (15) SEQ ID NOs: 1, 35, 62, 92, 114 and 133, respectively; (16) SEQ ID NOs: 4, 27, 70, 95, 119 and 139, respectively; (17) SEQ ID NOs: 4, 27, 71, 96, 116 and 136, respectively; (18) SEQ ID NOs: 10, 36, 72, 97, 120 and 140, respectively; (19) SEQ ID NOs: 4, 37, 73, 98, 121 and 141, respectively; (20) SEQ ID NOs: 11, 38, 74, 99, 122 and 142, respectively; (21) SEQ ID NOs: 12, 39, 75, 100, 123 and 143, respectively; (22) SEQ ID NOs: 13, 40, 76, 101, 124 and 144, respectively; (23) SEQ ID NOs: 14, 41, 77, 97, 120 and 145, respectively; (24) SEQ ID NOs: 5, 42, 64, 92, 114 and 133, respectively; (25) SEQ ID NOs: 5, 43, 78, 102, 115 and 146, respectively; (26) SEQ ID NOs: 15, 44, 79, 103, 115 and 147, respectively; (27) SEQ ID NOs: 8, 45, 80, 104, 125 and 148, respectively; (28) SEQ ID NOs: 16, 46, 81, 104, 125 and 148, respectively; (29) SEQ ID NOs: 2, 47, 61, 93, 115 and 149, respectively; (30) SEQ ID NOs: 17, 48, 82, 105, 126 and 150, respectively; (31) SEQ ID NOs: 11, 38, 83, 106, 116 and 142, respectively; (32) SEQ ID NOs: 10, 49, 84, 97, 120 and 151, respectively; (33) SEQ ID NOs: 2, 50, 61, 93, 115 and 134, respectively; (34) SEQ ID NOs: 10, 36, 85, 97, 120 and 152, respectively; (35) SEQ ID NOs: 5, 32, 86, 92, 114 and 133, respectively; (36) SEQ ID NOs: 18, 51, 87, 103, 115 and 147, respectively; (37) SEQ ID NOs: 2, 52, 88, 103, 115 and 149, respectively; (38) SEQ ID NOs: 19, 53, 89, 107, 127 and 153, respectively; (39) SEQ ID NOs: 5, 32, 64, 92, 114 and 133, respectively; (40) SEQ ID NOs: 20, 54, 90, 108, 128 and 154, respectively; (41) SEQ ID NOs: 2, 47, 61, 93, 115 and 134, respectively; (42) SEQ ID NOs: 4, 27, 71, 109, 116 and 136, respectively; (43) SEQ ID NOs: 21, 55, 91, 110, 129 and 155, respectively; (44) SEQ ID NOs: 21, 56, 91, 111, 130 and 156, respectively; (45) SEQ ID NOs: 21, 55, 346, 110, 129 and 155, respectively; (46) SEQ ID NOs: 22, 57, 347, 112, 131 and 157, respectively; (47) SEQ ID NOs: 21, 58, 91, 111, 132 and 158, respectively; (48) SEQ ID NOs: 21, 59, 91, 111, 130 and 159, respectively; (49) SEQ ID NOs: 2, 47, 61, 113, 115 and 134, respectively; or (50) SEQ ID NOs: 249, 250, 251, 252, 253 and 254, respectively. Also provided are variants of these antibodies or antigen-binding portions that comprise up to about 3 amino acid substitutions (e.g., one, two, or three amino acid substitutions) in each of the CDRs.

The isolated monoclonal antibody, or the antigen-binding portion thereof, of the present disclosure may comprise a heavy chain variable region which may comprise an amino acid sequence having at least 85%, 86%, 87%, 88%, 89%, 90%, 91%, 92%, 93%, 94%, 95%, 96%, 97%, 98%, 99% or 100% identity to any one of SEQ ID NOs: 160 to 209, 255, 325, 260 to 296 and 326 to 335. The isolated monoclonal antibody, or the antigen-binding portion thereof, of the present disclosure may comprise a light chain variable region which may comprise an amino acid sequence having at least 85%, 86%, 87%, 88%, 89%, 90%, 91%, 92%, 93%, 94%, 95%, 96%, 97%, 98%, 99% or 100% identity to any one of SEQ ID NOs: 210 to 247, 256, 297 to 324 and 336 to 345. The isolated monoclonal antibody, or the antigen-binding portion thereof, of the present disclosure may comprise (a) a heavy chain variable region which may comprise an amino acid sequence having at least 85%, 86%, 87%, 88%, 89%, 90%, 91%, 92%, 93%, 94%, 95%, 96%, 97%, 98%, 99% or 100% identity to any one of SEQ ID NOs: 160 to 209, 255, 325, 260 to 296 and 326 to 335 and/or (b) a light chain variable region which may comprise an amino acid sequence having at least 85%, 86%, 87%, 88%, 89%, 90%, 91%, 92%, 93%, 94%, 95%, 96%, 97%, 98%, 99% or 100% identity to any one of SEQ ID NOs: 210 to 247, 256, 297 to 324 and 336 to 345.

The isolated monoclonal antibody, or the antigen-binding portion thereof, of the present disclosure may comprise a heavy chain variable region and a light chain variable region which may comprise an amino acid sequences having at least 85%, 86%, 87%, 88%, 89%, 90%, 91%, 92%, 93%, 94%, 95%, 96%, 97%, 98%, 99% or 100% identity to (1) SEQ ID NOs: 160 and 210, respectively; (2) SEQ ID NOs: 161 and 211, respectively; (3) SEQ ID NOs: 162 and 212, respectively; (4) SEQ ID NOs: 163 and 210, respectively; (5) SEQ ID NOs: 164 and 213, respectively; (6) SEQ ID NOs: 165 and 210, respectively; (7) SEQ ID NOs: 166 and 214, respectively; (8) SEQ ID NOs: 167 and 215, respectively; (9) SEQ ID NOs: 168 and 216, respectively; (10) SEQ ID NOs: 169 and 217, respectively; (11) SEQ ID NOs: 170 and 218, respectively; (12) SEQ ID NOs: 171 and 210, respectively; (13) SEQ ID NOs: 172 and 210, respectively; (14) SEQ ID NOs: 173 and 219, respectively; (15) SEQ ID NOs: 174 and 210, respectively; (16) SEQ ID NOs: 175 and 220, respectively; (17) SEQ ID NOs: 176 and 221, respectively; (18) SEQ ID NOs: 177 and 222, respectively; (19) SEQ ID NOs: 178 and 223, respectively; (20) SEQ ID NOs: 179 and 224, respectively; (21) SEQ ID NOs: 180 and 225, respectively; (22) SEQ ID NOs: 181 and 226, respectively; (23) SEQ ID NOs: 182 and 227, respectively; (24) SEQ ID NOs: 183 and 210, respectively; (25) SEQ ID NOs: 184 and 228, respectively; (26) SEQ ID NOs: 185 and 229, respectively; (27) SEQ ID NOs: 186 and 230, respectively; (28) SEQ ID NOs: 187 and 230, respectively; (29) SEQ ID NOs: 188 and 231, respectively; (30) SEQ ID NOs: 189 and 232, respectively; (31) SEQ ID NOs: 190 and 233, respectively; (32) SEQ ID NOs: 191 and 234, respectively; (33) SEQ ID NOs: 192 and 235, respectively; (34) SEQ ID NOs: 193 and 236, respectively; (35) SEQ ID NOs: 194 and 212, respectively; (36) SEQ ID NOs: 195 and 229, respectively; (37) SEQ ID NOs: 196 and 237, respectively; (38) SEQ ID NOs: 197 and 248, respectively; (39) SEQ ID NOs: 198 and 215, respectively; (40) SEQ ID NOs: 199 and 238, respectively; (41) SEQ ID NOs: 200 and 239, respectively; (42) SEQ ID NOs: 201 and 239, respectively; (43) SEQ ID NOs: 202 and 240, respectively; (44) SEQ ID NOs: 203 and 241, respectively; (45) SEQ ID NOs: 204 and 242, respectively; (46) SEQ ID NOs: 205 and 243, respectively; (47) SEQ ID NOs: 206 and 242, respectively; (48) SEQ ID NOs: 207 and 244, respectively; (49) SEQ ID NOs: 208 and 245, respectively; (50) SEQ ID NOs: 209 and 246, respectively; (51) SEQ ID NOs: 188 and 247, respectively; or (52) SEQ ID NOs: 255 and 256, respectively.

The isolated monoclonal antibody, or the antigen-binding portion thereof, of the present disclosure may comprise a heavy chain variable region and a light chain variable region which may comprise an amino acid sequences having at least 85%, 86%, 87%, 88%, 89%, 90%, 91%, 92%, 93%, 94%, 95%, 96%, 97%, 98%, 99% or 100% identity to (1) SEQ ID NOs:260 and 297, respectively; (2) SEQ ID NOs: 260 and 298, respectively; (3) SEQ ID NOs: 260 and 299, respectively; (4) SEQ ID NOs: 261 and 297, respectively; (5) SEQ ID NOs: 261 and 298, respectively; (6) SEQ ID NOs: 261 and 299, respectively; (7) SEQ ID NOs: 262 and 297, respectively; (8) SEQ ID NOs: 262 and 298, respectively; (9) SEQ ID NOs: 262 and 299, respectively; (10) SEQ ID NOs: 263 and 300, respectively; (11) SEQ ID NOs: 263 and 301, respectively; (12) SEQ ID NOs: 263 and 302, respectively; (13) SEQ ID NOs: 264 and 300, respectively; (14) SEQ ID NOs: 264 and 301, respectively; (15) SEQ ID NOs: 264 and 302, respectively; (16) SEQ ID NOs: 265 and 300, respectively; (17) SEQ ID NOs: 266 and 303, respectively; (18) SEQ ID NOs: 267 and 303, respectively; (19) SEQ ID NOs: 268 and 303, respectively; (20) SEQ ID NOs: 269 and 303, respectively; (21) SEQ ID NOs: 270 and 304, respectively; (22) SEQ ID NOs: 271 and 305, respectively; (23) SEQ ID NOs: 325 and 324, respectively; (24) SEQ ID NOs: 272 and 306, respectively; (25) SEQ ID NOs: 272 and 307, respectively; (26) SEQ ID NOs: 272 and 308, respectively; (27) SEQ ID NOs: 272 and 309, respectively; (28) SEQ ID NOs: 273 and 306, respectively; (29) SEQ ID NOs: 273 and 307, respectively; (30) SEQ ID NOs: 273 and 308, respectively; (31) SEQ ID NOs: 273 and 309, respectively; (32) SEQ ID NOs: 274 and 310, respectively; (33) SEQ ID NOs: 274 and 311, respectively; (34) SEQ ID NOs: 275 and 310, respectively; (35) SEQ ID NOs: 275 and 311, respectively; (36) SEQ ID NOs: 275 and 312, respectively; (37) SEQ ID NOs: 276 and 310, respectively; (38) SEQ ID NOs: 276 and 311, respectively; (39) SEQ ID NOs: 276 and 312, respectively; (40) SEQ ID NOs: 277 and 313, respectively; (41) SEQ ID NOs: 276 and 314, respectively; (42) SEQ ID NOs: 276 and 315, respectively; (43) SEQ ID NOs: 276 and 316, respectively; (44) SEQ ID NOs: 276 and 317, respectively; (45) SEQ ID NOs: 281 and 310, respectively; (46) SEQ ID NOs: 282 and 310, respectively; (47) SEQ ID NOs: 278 and 310, respectively; (48) SEQ ID NOs: 279 and 310, respectively; (49) SEQ ID NOs: 280 and 310, respectively; (50) SEQ ID NOs: 280 and 311, respectively; (51) SEQ ID NOs: 283 and 319, respectively; (52) SEQ ID NOs: 284 and 319, respectively; (53) SEQ ID NOs: 283 and 318, respectively; (54) SEQ ID NOs: 284 and 318, respectively; (55) SEQ ID NOs: 288 and 310, respectively; (56) SEQ ID NOs: 290 and 310, respectively; (57) SEQ ID NOs: 291 and 310, respectively; (58) SEQ ID NOs: 288 and 311, respectively; (59) SEQ ID NOs: 291 and 311, respectively; (60) SEQ ID NOs: 288 and 312, respectively; (61) SEQ ID NOs: 285 and 320, respectively; (62) SEQ ID NOs: 286 and 320, respectively; (63) SEQ ID NOs: 287 and 320, respectively; (64) SEQ ID NOs: 292 and 323, respectively; (65) SEQ ID NOs: 293 and 321, respectively; (66) SEQ ID NOs: 293 and 322, respectively; (67) SEQ ID NOs: 293 and 323, respectively; (68) SEQ ID NOs: 294 and 321, respectively; (69) SEQ ID NOs: 294 and 322, respectively; (70) SEQ ID NOs: 294 and 323, respectively; (71) SEQ ID NOs: 295 and 321, respectively; (72) SEQ ID NOs: 296 and 322, respectively; (73) SEQ ID NOs: 326 and 336, respectively; (74) SEQ ID NOs: 326 and 337, respectively; (75) SEQ ID NOs: 327 and 338, respectively; (76) SEQ ID NOs: 328 and 338, respectively; (77) SEQ ID NOs: 329 and 341, respectively; (78) SEQ ID NOs: 330 and 339, respectively; (79) SEQ ID NOs: 330 and 340, respectively; (80) SEQ ID NOs: 327 and 337, respectively; (81) SEQ ID NOs: 331 and 342, respectively; (82) SEQ ID NOs: 335 and 337, respectively; (83) SEQ ID NOs: 332 and 343, respectively; (84) SEQ ID NOs: 333 and 345, respectively; (85) SEQ ID NOs: 334 and 345, respectively; or (86) SEQ ID NOs: 334 and 344, respectively.

The isolated monoclonal antibody, or the antigen-binding portion thereof, of the present disclosure may comprise a heavy chain variable region and a light chain variable region which may comprise an amino acid sequences having at least 85%, 86%, 87%, 88%, 89%, 90%, 91%, 92%, 93%, 94%, 95%, 96%, 97%, 98%, 99% or 100% identity to (1) SEQ ID NOs: 261 and 298, respectively; (2) SEQ ID NOs: 267 and 303, respectively; (3) SEQ ID NOs: 276 and 314, respectively; (4) SEQ ID NOs: 279 and 310, respectively; (5) SEQ ID NOs: 291 and 310, respectively; (6) SEQ ID NOs: 285 and 320, respectively; (7) SEQ ID NOs: 287 and 320, respectively; (8) SEQ ID NOs: 294 and 323, respectively; (9) SEQ ID NOs: 295 and 321, respectively; (10) SEQ ID NOs: 335 and 337, respectively; or (11) SEQ ID NOs: 333 and 345, respectively.

The isolated monoclonal antibody, or the antigen-binding portion thereof, of the present disclosure may comprise a heavy chain constant region and/or a light chain constant region, wherein the C terminus of the heavy chain variable region is linked to the N terminus of the heavy chain constant region, and the C terminus of the light chain variable region is linked to the N terminus of the light chain constant region. The heavy chain constant region may be with weak or no FcR/complement system binding affinity/capacity, such as an IgG4 constant region, an IgG1 or IgG2 constant region genetically modified to have reduced binding affinity/capacity to FcRs and proteins from the complement system, or a fragment thereof such as the one with CH2 and CH3 domains. In one embodiment, the heavy chain constant region is a human IgG4 constant region having the amino acid sequence of SEQ ID NO: 257, or a fragment thereof. The light chain constant region may be a kappa constant region, e.g., a human kappa constant region having the amino acid sequence of SEQ ID NO: 258, or a fragment thereof.

The isolated monoclonal antibody, or the antigen-binding portion thereof, of the present disclosure may be mouse, chimeric or humanized.

The isolated monoclonal antibody, or the antigen-binding portion thereof, of the disclosure, (i) binds to e.g., human or monkey Ang2 at comparable or higher binding affinity/activity as compared to prior art anti-Ang2 antibodies such as ABTAA; (ii) inhibits Ang2 binding to Tie2 at comparable or higher activity as compared to prior art anti-Ang2 antibodies such as ABTAA; (iii) activates Tie2 signaling at comparable or higher activity as compared to prior art anti-Ang2 antibodies such as ABTAA; and/or (iv) blocks Ang2 binding to integrines.

In another aspect, the disclosure also provides a bispecific molecule which may comprise the antibody, or the antigen-binding portion thereof, of the disclosure, linked to a second functional moiety (e.g., a second antibody or antigen-binding portion thereof) having a different binding specificity than the antibody, or antigen-binding portion thereof, such as one binding to VEGF.

A nucleic acid molecule encoding the antibody, or the antigen-binding portion thereof, of the disclosure is also encompassed by the disclosure, as well as an expression vector which may comprise such the nucleic acid molecule and a host cells which may comprise the expression vector. A method for preparing the antibody or the antigen-binding portion thereof of the disclosure using the host cell is also provided, which may comprise steps of (i) expressing the antibody or the antigen-binding portion thereof in the host cell and (ii) isolating the antibody or the antigen-binding portion thereof from the host cell or its cell culture.

The present disclosure also provides a composition which may comprise the antibody or the antigen-binding portion thereof, or the bispecific molecule, of the disclosure, and a pharmaceutically acceptable carrier. In some embodiments, the composition may comprise more than one antibody or antigen-binding portion thereof of the disclosure that bind different epitopes on the Ang2 protein. In some embodiments, the composition may comprise vectors expressing more than one antibody or antigen-binding portion thereof of the disclosure. In some embodiments, the composition may comprise bispecific molecules with more than one antibody or antigen-binding portion thereof of the disclosure.

In another aspect, the disclosure provides a method for stabilizing the vasculature or reducing vascular inflammation in a subject in need thereof, comprising administering to the subject a therapeutically effective amount of the composition of the disclosure.

In yet another aspect, the disclosure provides a method for treating an Ang2 related disease in a subject in need thereof, comprising administering to the subject a therapeutically effective amount of the composition of the disclosure.

In certain embodiments, the disease is cancer. In certain embodiments, the cancer is a solid tumor, including, but not limited to, ovarian cancer, lung cancer (e.g., Lewis lung carcinoma, small cell lung cancer, and non-small cell lung cancer), mammary cancer, cervical cancer, and kaposi's sarcoma, whether original or metastatic. The subject may be further administered with an anti-tumor agent, such as an anti-VEGF antibody, an anti-PD-1 antibody, an anti-PD-L1 antibody, an anti-CTLA-4 antibody, or an anti-LAG-3 antibody. In certain embodiments, the subject is human.

In certain embodiments, the disease is an inflammatory disease, such as sepsis, macular edema, diabetic retinopathy and age-related macular degeneration (e.g., neovascular age-related macular degeneration). The subject may be further administered with an anti-inflammatory agent, such as an anti-VEGF antibody. In certain embodiments, the subject is human.

In certain embodiments, the disease is an infectious disease, such as pneumonias caused by infection of bacteria, viruses or mycoplasma. The subject may be further administered with an anti-infectious agent, such as an anti-bacterial agent, an anti-virus agent and an anti-mycoplasma agent. In certain embodiments, the subject is human.

The disclosure further provides a method for diagnosis of a disease related with excessive Ang2 in a subject in need thereof, comprising contacting the antibody or antigen binding portion thereof of the disclosure with a sample collected from the subject, and determining the binding level as compared to that in a control sample. In certain embodiments, the sample is serum or a lesion tissue. In certain embodiments, the subject is human.

Other features and advantages of the instant disclosure will be apparent from the following detailed description and examples which should not be construed as limiting. The contents of all references, GenBank entries, patents and published patent applications cited throughout this application are expressly incorporated herein by reference.

Accordingly, it is an object of the invention not to encompass within the invention any previously known product, process of making the product, or method of using the product such that Applicants reserve the right and hereby disclose a disclaimer of any previously known product, process, or method. It is further noted that the invention does not intend to encompass within the scope of the invention any product, process, or making of the product or method of using the product, which does not meet the written description and enablement requirements of the USPTO (35 U.S.C. § 112, first paragraph) or the EPO (Article 83 of the EPC), such that Applicants reserve the right and hereby disclose a disclaimer of any previously described product, process of making the product, or method of using the product. It may be advantageous in the practice of the invention to be in compliance with Art. 53(c) EPC and Rule 28(b) and (c) EPC. All rights to explicitly disclaim any embodiments that are the subject of any granted patent(s) of applicant in the lineage of this application or in any other lineage or in any prior filed application of any third party is explicitly reserved. Nothing herein is to be construed as a promise.

It is noted that in this disclosure and particularly in the claims and/or paragraphs, terms such as “comprises”, “comprised”, “comprising” and the like can have the meaning attributed to it in U.S. patent law; e.g., they can mean “includes”, “included”, “including”, and the like; and that terms such as “consisting essentially of” and “consists essentially of” have the meaning ascribed to them in U.S. patent law, e.g., they allow for elements not explicitly recited, but exclude elements that are found in the prior art or that affect a basic or novel characteristic of the invention.

### BRIEF DESCRIPTION OF THE DRAWINGS

The following detailed description, given by way of example, but not intended to limit the invention solely to the specific embodiments described, may best be understood in conjunction with the accompanying drawings.

FIGS. 1A-1C show the binding activities of mouse antibodies of the disclosure to human (A, C) and mouse Ang2 proteins (B) in an indirect ELISA.

FIGS. 2A-2C show the binding activities of mouse antibodies of the disclosure to cells expressing membrane-anchored human (A), cyno (B) and mouse (C) Ang2 proteins in a FACS assay.

FIGS. 3A-3E show the effects of mouse antibodies of the disclosure on Tie2 signaling activation in an in vitro phos-Akt bioassay. Antibodies 4D8F2, 5A7B9, 5F7D7 and 46F3H4 (A), 50H8G3 (B), 17F3E8, 7F10B2 and 31E2D4 (C), 50H8E5C5, 25C5D6F3 15H10D12 (D), and 82B10C1 (E) had much lower EC₅₀ and higher spans.

FIGS. 4A-4C shows the mouse antibodies' capacities of blocking Ang2-Tie2 interaction in a cell based blockade assay. Antibodies 45C5A1 (A), 53E8B2, 60C4B9, 73H11G10, 85E10B9 (B), 55D3F10 and 98C7H11 (C) blocked Ang2 binding to Tie2.

FIG. 5 shows the effects of chimeric antibodies of the disclosure on Tie2 signaling activation in an in vitro phos-Akt bioassay.

FIGS. 6A-6C show the effects of humanized antibodies 46H3H3 (A), 42C3A4 (B), and 4D8F2 (C) on Tie2 signaling activation in an in vitro phos-Akt bioassay.

FIGS. 7A-7B show the effects of humanized antibodies 42A2A6 (A) and 5A7B9 (FIG. B), on Tie2 signaling activation in an in vitro phos-Akt bioassay.

FIGS. 8A-8C show the effects of humanized antibodies 5A7B9 (A & B) and 5F7D7 (C) on Tie2 signaling activation in an in vitro phos-Akt bioassay.

FIG. 9 shows the effects of 7F10B2 humanized antibodies of the disclosure on Tie2 signaling activation in an in vitro phos-Akt bioassay.

FIGS. 10A-10C show the capacities of humanized antibodies blocking Ang2-Tie2 interaction in a cell based blockade assay; 53E8B2 (A), 85E10B9 (B), and 55D3F10 (C).

FIGS. 11A-11B shows the binding affinity of humanized antibodies of the disclosure to human Ang2 protein by ELISA.

### DETAILED DESCRIPTION OF THE INVENTION

To ensure that the present disclosure may be more readily understood, certain terms are set forth throughout the detailed description.

The terms “a” and “an” as used herein refer to one or to more than one (i.e., to at least one) of the grammatical object of the article. By way of example, “an antibody” means one antibody or more than one antibody.

The term “Ang2” refers angiopoietin-2, a growth factor belong to the antiopoietin/Tie signaling pathway, one of the main pathways involved in angiogenesis. The term “Ang2” may comprise variants, isoforms, homologs, orthologs and paralogs. For example, an antibody specific for a human Ang2 protein may, in certain cases, cross-react with an Ang2 protein from a species other than human, such as monkey. In other embodiments, an antibody specific for a human Ang2 protein may be completely specific for the human Ang2 protein and exhibit no cross-reactivity to other species or of other types, or may cross-react with Ang2 from certain other species but not all other species.

The term “human Ang2” refers to an Ang2 protein having an amino acid sequence from a human, such as the amino acid sequence of human Ang2 having a Genbank accession number of AAB63190.1 (Maisonpierre P C et al., (1997) Science 277(5322): 55-60). The terms “monkey Ang2” or “cynomolgus Ang2” refer to an Ang2 protein having an amino acid sequence from macaca mulatta, such as the amino acid sequence having NCBI Reference No. XP_001097757.2. The term “mouse Ang2” refers to an Ang2 protein having an amino acid sequence from a mouse, such as the amino acid sequence of mouse Ang2 having a Genbank accession number of AAB63189.1 (Maisonpierre P C et al., (1997) supra).

The term “Tie2”, also known as angiopoietin-1 receptor or CD202B, is a tyrosine kinase with Ig and EGF homology domains. Tie2 is encoded by the TEK gene in human. The term “Ang2” may comprise variants, isoforms, homologs, orthologs and paralogs. The term “human Tie2” refers to a Tie2 protein having an amino acid sequence from a human, such as the amino acid of human Tie2 having a NCBI Reference Sequence: NP_001277006.1 (Drost C C et al., (2019) Thromb. Haemost. 119(11): 1827-1838).

The term “antibody” as used herein refers to an immunoglobulin molecule that recognizes and specifically binds a target, such as Ang2, through at least one antigen-binding site wherein the antigen-binding site is usually within the variable region of the immunoglobulin molecule. As used herein, the term encompasses intact polyclonal antibodies, intact monoclonal antibodies, single-chain Fv (scFv) antibodies, heavy chain antibodies (HCAbs), light chain antibodies (LCAbs), multispecific antibodies, bispecific antibodies, monospecific antibodies, monovalent antibodies, fusion proteins comprising an antigen-binding site of an antibody, and any other modified immunoglobulin molecule comprising an antigen-binding site (e.g., dual variable domain immunoglobulin molecules) as long as the antibodies exhibit the desired biological activity. Antibodies also include, but are not limited to, mouse antibodies, chimeric antibodies, humanized antibodies, and human antibodies. An antibody can be any of the five major classes of immunoglobulins: IgA, IgD, IgE, IgG, and IgM, or subclasses (isotypes) thereof (e.g., IgG1, IgG2, IgG3, IgG4, IgA1 and IgA2), based on the identity of their heavy-chain constant domains referred to as alpha, delta, epsilon, gamma, and mu, respectively. The different classes of immunoglobulins have different and well-known subunit structures and three-dimensional configurations. Antibodies can be naked or conjugated to other molecules, including but not limited to, toxins and radioisotopes. Unless expressly indicated otherwise, the term “antibody” as used herein include “antigen-binding portion” of the intact antibodies. An IgG is a glycoprotein which may comprise two heavy (H) chains and two light (L) chains inter-connected by disulfide bonds. Each heavy chain may be comprised of a heavy chain variable region (abbreviated herein as VH) and a heavy chain constant region. The heavy chain constant region may be comprised of three domains, C_{H1}, C_{H2} and C_{H3}. Each light chain may be comprised of a light chain variable region (abbreviated herein as V_{L}) and a light chain constant region. The light chain constant region may be comprised of one domain, C_{L}. The V_{H} and V_{L} regions can be further subdivided into regions of hypervariability, termed complementarity determining regions (CDR), interspersed with regions that are more conserved, termed framework regions (FR). Each V_{H} and V_{L} is composed of three CDRs and four FRs, arranged from amino-terminus to carboxy-terminus in the following order: FR1, CDR1, FR2, CDR2, FR3, CDR3, FR4. The variable regions of the heavy and light chains contain a binding domain that interacts with an antigen. The constant regions of the antibodies can mediate the binding of the immunoglobulin to host tissues or factors, including various cells of the immune system (e.g., effector cells) and the first component (C1q) of the classical complement system.

The term “antigen-binding portion” or “antigen-binding fragment” as used in connection with an antibody refers to one or more fragments of an antibody that retain the ability to specifically bind to an antigen (e.g., Ang2). It has been shown that the antigen-binding function of an antibody can be performed by fragments of a full-length antibody. Examples of binding fragments encompassed within the term “antigen-binding portion” of an antibody include, but not limited to, (i) a Fab fragment, a monovalent fragment consisting of the V_{L}, V_{H}, C_{L} and C_{H1} domains; (ii) a F(ab′)₂ fragment, a bivalent fragment comprising two Fab fragments linked by a disulfide bridge at the hinge region; (iii) a Fd fragment consisting of the V_{H} and C_{H1} domains; (iv) a Fv fragment consisting of the V_{L} and V_{H} domains of a single arm of an antibody, (v) a dAb fragment (Ward et al., (1989) Nature 341:544-546), which consists of a V_{H} domain; (vi) an isolated complementarity determining region (CDR); and (viii) a nanobody, a heavy chain variable region containing a single variable domain and two constant domains. Furthermore, although the two domains of the Fv fragment, V_{L} and V_{H}, are coded by separate genes, they can be joined, using recombinant methods, by a synthetic linker that enables them to be made as a single protein chain in which the V_{L} and V_{H} regions pair to form monovalent molecules (known as single chain Fv (scFv); see e.g., Bird et al., (1988) Science 242:423-426; and Huston et al., (1988) Proc. Natl. Acad. Sci. USA 85:5879-5883). Such single chain antibodies are also intended to be encompassed within the term “antigen-binding portion” of an antibody. These antibody fragments are obtained using conventional techniques known to those with skill in the art, and the fragments are screened for utility in the same manner as are intact antibodies. The term “single chain variable fragment” or “scFv” refers to a fusion protein of the heavy chain variable region and light chain variable region of immunoglobulins, connected with a short linker peptide of ten to twenty-five amino acids. The linker is usually rich in glycine for flexibility, as well as serine or threonine for solubility. The scFv retains the specificity of the original immunoglobulin. The scFvs can be linkered by linkers of different lengths to form di-scFvs, diabodies, tri-scFvs, triabodies, or tetrabodies, which may show specificity to one or more antigens.

The term “Fc region” of an antibody is the tail region of an antibody that interacts with Fc receptors and some proteins of the complement system to activate the immune system. The IgG, IgA and IgD Fc region is composed of two identical fragments derived from the second and third constant domains (C_{H2} and C_{H3}) of the antibody's heavy chains, while the IgM and IgE Fc regions contain three heavy chain constant domains (C_{H} domains 2-4). The Fc region may bind to the complement component C1q to activate the classical complement cascade, may bind to the Fc receptors on phagocytes (i.e., macrophages, granulocytes and dendritic cells) to induce phagocytosis of cells bound by the antibodies, may bind to the Fc receptors of immune effector cells (mainly natural killer cells) to induce release of cytotoxic granules from the immune effector cells, which cause the death of the antibody-coated cells, and may bind to the Fc receptor of the antigen-presenting cells such as dendritic cells to induce humoral and cellular antiviral immune responses. In the present disclosure, the Fc region is genetically modified to have reduced binding affinity/activity to FcR and complement system proteins.

The term “binding affinity” as used herein generally refers to the strength of the sum total of noncovalent interactions between an antibody or an antigen-binding portion thereof of the disclosure and a target molecule such as Ang2. The binding of the antibody or antigen-binding portion thereof and the target molecule is reversible, and the binding affinity is typically reported as an equilibrium dissociation constant (K_{D}). K_{D} is the ratio of a dissociation rate (k_{off} or k_{d}) to the association rate (kₒₙ or kₐ). The lower the K_{D} of a binding pair, the higher the affinity. A variety of methods of measuring binding affinity are known in the art, any of which can be used for purposes of the present disclosure. Specific illustrative embodiments include the following. In one embodiment, the “K_{D}” or “K_{D} value” can be measured by assays known in the art, for example by a binding assay. The K_{D} may be measured in a radiolabeled antigen binding assay (RIA) (Chen, et al., (1999) J. Mol Biol 293:865-881). The K_{D} or K_{D} value may also be measured by using surface plasmon resonance assays by Biacore, using, for example, a BIAcore™-2000 or a BIAcore™-3000 BIAcore, Inc., Piscataway, NJ), or by biolayer interferometry using, for example, the OctetQK384 system (ForteBio, Menlo Park, CA). When a target molecule containing multiple epitopes come in contact with an antibody or antigen-binding portion containing multiple binding sites that bind the target molecule, the interaction of the binding molecule with the target molecule at one site may increase the probability of a reaction at a second site. The strength of such multiple interactions between a multivalent antibody and an antigen is called avidity. For example, high avidity can compensate for low affinity as is sometimes found for pentameric IgM antibodies, which can have a lower affinity than IgG, but the high avidity of IgM, resulting from its multivalence, enables it to bind antigen effectively.

The term “specifically binds” as used herein, means that an antibody or an antigen-binding portion thereof interacts more frequently, more rapidly, with greater duration, with greater affinity, or with some combination of the above to an antigen, or an epitope than alternative substances, including related and unrelated proteins. An antibody or an antigen-binding portion thereof that specifically binds a target molecule (e.g. Ang2) may be identified, for example, by immunoassays, ELISAs, SPR (e.g., Biacore), or other techniques known to those of skill in the art. Typically, a specific reaction will be at least twice background signal or noise and can be more than 10 times background. See, e.g., Paul, ed., 1989, Fundamental Immunology Second Edition, Raven Press, New York at pages 332-336 for a discussion regarding antibody specificity. An antibody or an antigen-binding portion thereof that specifically binds a target molecule may bind the target molecule at a higher affinity than its affinity for a different molecule. In some embodiments, an antibody or an antigen-binding portion thereof that specifically binds a target molecule may bind the target molecule with an affinity that is at least 20 times greater, at least 30 times greater, at least 40 times greater, at least 50 times greater, at least 60 times greater, at least 70 times greater, at least 80 times greater, at least 90 times greater, or at least 100 times greater, than its affinity for a different molecule. In some embodiments, an antibody or an antigen-binding portion thereof that specifically binds a particular target molecule may bind a different molecule at such a low affinity that the binding cannot be detected using an assay described herein or otherwise known in the art. In some embodiments, “specifically binds” means, for instance, that an antibody or an antigen-binding portion thereof binds a molecule target with a K_{D} of about 5.0E-08 or less. Because of the sequence identity between homologous proteins in different species, an antibody or an antigen-binding portion thereof that specifically recognizes a target molecule may cross-react with the target in other species. It is understood that an antibody or an antigen-binding portion thereof that specifically binds a first target may or may not specifically bind a second target. As such, “specific binding” does not necessarily require (although it can include) exclusive binding, i.e., binding to a single target. Thus, an antibody or an antigen-binding portion thereof, in some embodiments, specifically bind more than one target. For example, an antibody or an antigen-binding portion thereof may, in certain instances, comprise two identical antigen-binding sites, each of which specifically binds a same epitope.

An “isolated antibody”, as used herein, is intended to refer to an antibody that is substantially free of other antibodies having different antigenic specificities (e.g., an isolated antibody that specifically binds the Ang2 is substantially free of antibodies that specifically bind antigens other than Ang2). An isolated antibody that specifically binds the Ang2 may, however, have cross-reactivity to other antigens, such as an Ang2 proteins from another species. Moreover, an isolated antibody can be substantially free of other cellular material and/or chemicals.

The term “monoclonal antibody” as used herein refers to an antibody obtained from a population of substantially homogeneous antibodies, i.e., the individual antibodies comprising the population are identical except for possible naturally occurring mutations and/or post-translation modifications (e.g., isomerizations, amidations) that may be present in minor amounts. Monoclonal antibodies are highly specific, being directed against a single antigenic site. In contrast to polyclonal antibody preparations which typically include different antibodies directed against different determinants (epitopes), each monoclonal antibody is directed against a single determinant on the antigen. In addition to their specificity, the monoclonal antibodies are advantageous in that they are synthesized by the hybridoma culture, uncontaminated by other immunoglobulins. The modifier “monoclonal” indicates the character of the antibody as being obtained from a substantially homogeneous population of antibodies, and is not to be construed as requiring production of the antibody by any particular method. For example, the monoclonal antibodies to be used in accordance with the present invention may be made by a variety of techniques, including, for example, the hybridoma method (e.g., Kohler and Milstein, Nature, 256:495-97 (1975); Hongo et al., Hybridoma, 14 (3): 253-260 (1995), Harlow et al., Antibodies: A Laboratory Manual, (Cold Spring Harbor Laboratory Press, 2^{nd} ed. 1988); Hammerling et al., in: Monoclonal Antibodies and T-Cell Hybridomas 563-681 (Elsevier, N.Y., 1981)), recombinant DNA methods (see, e.g., U.S. Pat. No. 4,816,567), phage-display technologies (see, e.g., Clackson et al., Nature, 352: 624-628 (1991); Marks et al., J. Mol. Biol. 222: 581-597 (1992); Sidhu et al., J. Mol. Biol. 338(2): 299-310 (2004); Lee et al., J. Mol. Biol. 340(5): 1073-1093 (2004); Fellouse, Proc. Natl. Acad. Sci. USA 101(34): 12467-12472 (2004); and Lee et al., J. Immunol. Methods 284(1-2): 119-132 (2004), and technologies for producing human or human-like antibodies in animals that have parts or all of the human immunoglobulin loci or genes encoding human immunoglobulin sequences (see. e.g., WO 1998/24893; WO 1996/34096; WO 1996/33735; WO 1991/10741; Jakobovits et al., Proc. Natl. Acad. Sci. USA 90: 2551 (1993); Jakobovits et al., Nature 362: 255-258 (1993); Bruggemann et al., Year in Immunol. 7:33 (1993); U.S. Pat. Nos. 5,545,807; 5,545,806; 5,569,825; 5,625,126; 5,633,425; and U.S. Pat. No. 5,661,016; Marks et al., Bio/Technology 10: 779-783 (1992); Lonberg et al., Nature 368: 856-859 (1994); Morrison, Nature 368: 812-813 (1994); Fishwild et al., Nature Biotechnol. 14: 845-851 (1996); Neuberger, Nature Biotechnol. 14: 826 (1996); and Lonberg and Huszar, Intern. Rev. Immunol. 13: 65-93 (1995).

The term “mouse antibody”, as used herein, is intended to include antibodies having variable regions in which both the framework and CDR regions are derived from mouse germline immunoglobulin sequences. Furthermore, if the antibody contains a constant region, the constant region also is derived from mouse germline immunoglobulin sequences. The mouse antibodies of the disclosure can include amino acid residues not encoded by mouse germline immunoglobulin sequences (e.g., mutations introduced by random or site-specific mutagenesis in vitro or by somatic mutation in vivo). However, the term “mouse antibody”, as used herein, is not intended to include antibodies in which CDR sequences derived from the germline of another mammalian species have been grafted onto mouse framework sequences.

The term “chimeric antibody” refers to an antibody made by combining genetic material from a nonhuman source with genetic material from a human being. Or more generally, a chimeric antibody is an antibody having genetic material from a certain species with genetic material from another species.

The term “humanized antibody”, as used herein, refers to an antibody from non-human species whose protein sequences have been modified to increase similarity to antibody variants produced naturally in humans.

The term “IC₅₀”, also known as half maximal inhibitory concentration, refers to the concentration of an antibody or an antigen-binding portion thereof which inhibits a specific biological or biochemical function or process, e.g., interaction of Ang2 with Tie2, by 50%, relative to the absence of the antibody or antigen-binding portion thereof.

The term “EC₅₀”, also known as half maximal effective concentration, refers to the concentration of an antibody or an antigen-binding portion thereof that gives half-maximal response, e.g., in an ELISA binding test.

The term “subject” includes any human or nonhuman animal. The term “nonhuman animal” includes all vertebrates, e.g., mammals and non-mammals, such as non-human primates, sheep, dogs, cats, cows, horses, chickens, amphibians, and reptiles, although mammals are preferred, such as non-human primates, sheep, dogs, cats, cows and horses.

The term “therapeutically effective amount” means an amount of the antibody or antigen-binding portions thereof of the present disclosure sufficient to prevent or reduce the symptoms associated with a disease or condition (such as cancer or sepsis) and/or lessen the severity of the disease or condition. A therapeutically effective amount is understood to be in context to the condition being treated, where the actual effective amount is readily discerned by those of skill in the art.

The term “treat” or “treating” as used herein in connection with a disease or a condition, or a subject having a disease or a condition refers to an action that suppresses, eliminates, reduces, and/or ameliorates a symptom, the severity of the symptom, and/or the frequency of the symptom associated with the disease or disorder being treated.

The term “administer,” “administering,” or “administration” as used herein refers to the act of delivering, or causing to be delivered, a therapeutic or a pharmaceutical composition to the body of a subject by a method described herein or otherwise known in the art. Administering a therapeutic or a pharmaceutical composition includes prescribing a therapeutic or a pharmaceutical composition to be delivered into the body of a patient. Exemplary forms of administration include oral dosage forms, such as tablets, capsules, syrups, suspensions; injectable dosage forms, such as intravenous (IV), intramuscular (IM), or intraperitoneal (IP); transdermal dosage forms, including creams, jellies, powders, or patches; buccal dosage forms; inhalation powders, sprays, suspensions, and rectal suppositories.

The terms percent “identity” as used herein in the context of two or more nucleic acids or polypeptides, refer to two or more sequences or subsequences that have a specified percentage of nucleotides or amino acid residues that are the same, when compared and aligned (introducing gaps, if necessary) for maximum correspondence, not considering any conservative amino acid substitutions as part of the sequence identity. The percent identity can be measured using sequence comparison software or algorithms or by visual inspection. Various algorithms and software that can be used to obtain alignments of amino acid or nucleotide sequences are well-known in the art. These include, but are not limited to, BLAST, ALIGN, Megalign, BestFit, GCG Wisconsin Package, and variants thereof.

As used herein, the term “conservative sequence modifications” is intended to refer to amino acid modifications that do not significantly affect or alter the binding characteristics of the antibody containing the amino acid sequence. Such conservative modifications include amino acid substitutions, additions and deletions. A “conservative amino acid substitution” is one in which one amino acid residue is replaced with another amino acid residue having a side chain with similar chemical characteristics. Families of amino acid residues having similar side chains have been generally defined in the art, including basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine). For example, substitution of a phenylalanine for a tyrosine is a conservative substitution. Generally, conservative substitutions in the sequences of the polypeptides, soluble proteins, and/or antibodies of the disclosure do not abrogate the binding of the polypeptide, soluble protein, or antibody containing the amino acid sequence, to the target binding site. Methods of identifying amino acid conservative substitutions which do not eliminate binding are well-known in the art.

The term “variant” as used herein refers to a different antibody or antigen-binding portion thereof that comprises one or more (such as, for example, about 1 to about 25, about 1 to about 20, about 1 to about 15, about 1 to about 10, or about 1 to about 5) amino acid substitutions, deletions, and/or additions as compared to a reference antibody or antigen-binding portion thereof, but remains the antigen binding affinity/capacity as the reference antibody or antigen-binding portion thereof has.

The term “vector” refers to a substance that is used to carry or include a nucleic acid sequences, including for example, in order to introduce a nucleic acid sequence into a host cell. Vectors applicable for use include, for example, expression vectors, plasmids, phage vectors, viral vectors, episomes and artificial chromosomes, which can include selection sequences or markers operable for stable integration into a host cell's chromosome. Additionally, the vectors can include one or more selectable marker genes and appropriate expression control sequences. Selectable marker genes that can be included, for example, provide resistance to antibiotics or toxins, complement auxotrophic deficiencies, or supply critical nutrients not in the culture media. Expression control sequences can include constitutive and inducible promoters, transcription enhancers, transcription terminators, and the like which are well known in the art. When two or more nucleic acid molecules are to be co-expressed (e.g. both an antibody heavy and light chain or an antibody V_{H} and V_{L}), both nucleic acid molecules can be inserted, for example, into a single expression vector or in separate expression vectors. For single vector expression, the encoding nucleic acids can be operationally linked to one common expression control sequence or linked to different expression control sequences, such as one inducible promoter and one constitutive promoter. The introduction of nucleic acid molecules into a host cell can be confirmed using methods well known in the art. It is understood by those skilled in the art that the nucleic acid molecules are expressed in a sufficient amount to produce a desired product (e.g. an antibody as described herein), and it is further understood that expression levels can be optimized to obtain sufficient expression using methods well known in the art.

As herein used, the term “macular degeneration” refers to a condition in which neovascularization abnormally grows, so causes macula damage and affects vision. Macular degeneration occurs mainly in over 50 years of age and is divided into non-exudative (dry type) or exudative (wet type). In particular, in the case of wet AMD, blindness can be caused. The cause of the AMD has not yet been clarified, but it is known that risk factors are age; and environmental factors including smoking, hypertension, obesity, genetic predisposition, excessive UV exposure, low serum antioxidant concentrations and the like.

As herein used, the term “macular edema” refers to the swelling of the macula of the retina, and the swelling occurs due to fluid leakage from the retinal blood vessels. Blood leaks from the weak blood vessel wall, enters the localized area of the retinal macula which is the color-sensing nerve ending and in which the retinal conic is abundant. The image is then faded to the right of the center or center of the center area. Visual acuity decreases gradually over several months. As herein used, the term “diabetic retinopathy” refers to a complication of the eye in which visual acuity is reduced due to disturbance of microcirculation of the retina due to peripheral circulatory disorder caused by diabetes. Initially, it can cause light problems of visual acuity, but eventually it can cause blindness. Diabetic retinopathy can occur in anyone with Type 1 diabetes or Type 2 diabetes.

Various aspects of the disclosure are described below in further detail.

The antibody or antigen-binding portion thereof of the disclosure specifically binds to the Ang2 protein or a fragment thereof at comparable or higher binding affinity/activity as compared to prior art anti-Ang2 antibodies such as ABTAA. The antibody or antigen-binding portion thereof may inhibit the binding of Ang2 to Tie2 at comparable or higher activity as compared to prior art anti-Ang2 antibodies such as ABTAA; activates Tie2 signaling at comparable or higher activity as compared to prior art anti-Ang2 antibodies such as ABTAA; and/or blocks Ang2 binding to integrins.

The antibody of the disclosure is a monoclonal mouse, chimeric or humanized antibody.

The amino acid sequence ID numbers of the heavy/light chain CDRs and variable regions of the antibodies or antigen-binding portions thereof of the disclosure are set forth in Table 1 below. The heavy chain variable region CDRs and the light chain variable region CDRs have been defined by the IMGT numbering system. However, as is well known in the art, CDR regions can also be determined by other systems such as Chothia, and Kabat, AbM, or Contact numbering system/method, based on heavy chain/light chain variable region sequences.

TABLE 1
Amino acid sequence ID numbers of heavy/light chain CDRs and heavy/light
chain variable regions of antibodies of the disclosure

| | | VH- | VH- | VH- | VL- | VL- | VL- | | |
|---|---|---|---|---|---|---|---|---|---|
| | mAb ID | CDR1 | CDR2 | CDR3 | CDR1 | CDR2 | CDR3 | VH | VL |
| 1 | 43F1C10-2 | 1 | 23 | 60 | 92 | 114 | 133 | 160 | 210 |
| 2 | 49E9G10 | 2 | 24 | 61 | 93 | 115 | 134 | 161 | 211 |
| 3 | 50B9C10 | 3 | 25 | 62 | 92 | 114 | 133 | 162 | 212 |
| 4 | 46F3H4 | 3 | 26 | 62 | 92 | 114 | 133 | 163 | 210 |
| 5 | 4D8F2 | 4 | 27 | 63 | 92 | 116 | 135 | 164 | 213 |
| 6 | 50H8G3 | 5 | 28 | 64 | 92 | 114 | 133 | 165 | 210 |
| 7 | 5A7B9 | 6 | 29 | 65 | 92 | 116 | 136 | 166 | 214 |
| 8 | 5F7D7 | 1 | 25 | 62 | 92 | 114 | 133 | 167 | 215 |
| 9 | 7F10B2 | 7 | 30 | 66 | 92 | 117 | 137 | 168 | 216 |
| 10 | 31E2D4 | 8 | 29 | 65 | 92 | 116 | 136 | 169 | 217 |
| 11 | 15H10D12 | 4 | 31 | 63 | 92 | 116 | 135 | 170 | 218 |
| 12 | 25C5D6F3 | 5 | 32 | 67 | 92 | 114 | 133 | 171 | 210 |
| 13 | 17F3E8 | 3 | 33 | 68 | 92 | 114 | 133 | 172 | 210 |
| 14 | 82B10C1 | 9 | 34 | 69 | 94 | 118 | 138 | 173 | 219 |
| 15 | 50H8E5C5 | 1 | 35 | 62 | 92 | 114 | 133 | 174 | 210 |
| 16 | 41E4F2 | 4 | 27 | 70 | 95 | 119 | 139 | 175 | 220 |
| 17 | 41F7H8 | 4 | 27 | 71 | 96 | 116 | 136 | 176 | 221 |
| 18 | 41H8A8 | 10 | 36 | 72 | 97 | 120 | 140 | 177 | 222 |
| 19 | 42A2A6 | 4 | 37 | 73 | 98 | 121 | 141 | 178 | 223 |
| 20 | 42C3A4 | 11 | 38 | 74 | 99 | 122 | 142 | 179 | 224 |
| 21 | 43B2A12 | 12 | 39 | 75 | 100 | 123 | 143 | 180 | 225 |
| 22 | 43C4A11 | 13 | 40 | 76 | 101 | 124 | 144 | 181 | 226 |
| 23 | 43D3A9 | 14 | 41 | 77 | 97 | 120 | 145 | 182 | 227 |
| 24 | 43F1C10 | 5 | 42 | 64 | 92 | 114 | 133 | 183 | 210 |
| 25 | 43G5G3 | 5 | 43 | 78 | 102 | 115 | 146 | 184 | 228 |
| 26 | 45C5A1 | 15 | 44 | 79 | 103 | 115 | 147 | 185 | 229 |
| 27 | 45C9F9 | 8 | 45 | 80 | 104 | 125 | 148 | 186 | 230 |
| 28 | 45C9F9-2 | 16 | 46 | 81 | 104 | 125 | 148 | 187 | 230 |
| 29 | 46H3H3 | 2 | 47 | 61 | 93 | 115 | 149 | 188 | 231 |
| 30 | 49C3B8 | 17 | 48 | 82 | 105 | 126 | 150 | 189 | 232 |
| 31 | 49E4C2 | 11 | 38 | 83 | 106 | 116 | 142 | 190 | 233 |
| 32 | 49E4F1 | 10 | 49 | 84 | 97 | 120 | 151 | 191 | 234 |
| 33 | 49G4D6 | 2 | 50 | 61 | 93 | 115 | 134 | 192 | 235 |
| 34 | 4D11H2 | 10 | 36 | 85 | 97 | 120 | 152 | 193 | 236 |
| 35 | 50B9C10-2 | 5 | 32 | 86 | 92 | 114 | 133 | 194 | 212 |
| 36 | 50C9C2 | 18 | 51 | 87 | 103 | 115 | 147 | 195 | 229 |
| 37 | 50F11D12 | 2 | 52 | 88 | 103 | 115 | 149 | 196 | 237 |
| 38 | 54E4A12 | 19 | 53 | 89 | 107 | 127 | 153 | 197 | 248 |
| 39 | 5F7D7-2 | 5 | 32 | 64 | 92 | 114 | 133 | 198 | 215 |
| 40 | 76B2E6 | 20 | 54 | 90 | 108 | 128 | 154 | 199 | 238 |
| 41 | 41A6A6 | 2 | 47 | 61 | 93 | 115 | 134 | 200 | 239 |
| 42 | 39C1C4 | 2 | 50 | 61 | 93 | 115 | 134 | 201 | 239 |
| 43 | 49B8G4 | 2 | 50 | 61 | 93 | 115 | 134 | 202 | 240 |
| 44 | 27H11B5 | 4 | 27 | 71 | 109 | 116 | 136 | 203 | 241 |
| 45 | 53E8B2 | 21 | 55 | 91 | 110 | 129 | 155 | 204 | 242 |
| 46 | 60C4B9 | 21 | 56 | 91 | 111 | 130 | 156 | 205 | 243 |
| 47 | 73H11G10 | 21 | 55 | 346 | 110 | 129 | 155 | 206 | 242 |
| 48 | 15B4C6 | 22 | 57 | 347 | 112 | 131 | 157 | 207 | 244 |
| 49 | 55D3F10 | 21 | 58 | 91 | 111 | 132 | 158 | 208 | 245 |
| 50 | 98C7H11 | 21 | 59 | 91 | 111 | 130 | 159 | 209 | 246 |
| 51 | 33A3E2 | 2 | 47 | 61 | 113 | 115 | 134 | 188 | 247 |
| 52 | 85E10B9 | 249 | 250 | 251 | 252 | 253 | 254 | 255 | 256 |

The antibodies of the disclosure may contain a heavy chain constant region, such as a human IgG4 heavy chain constant region having an amino acid sequence set forth in, e.g., SEQ ID NO: 257, and/or a light chain constant region such as human kappa constant region having an amino acid sequence set forth in, e.g., SEQ ID NO: 258. The antibodies or antigen-binding portions thereof of the disclosure may also contain other appropriate complete or partial heavy chain constant regions and/or complete or partial light chain constant regions.

The antibody of the disclosure may be a full-length antibody, a heavy chain antibody (HCAb), a Fab, a F(ab′)₂, a Fv, a scFv, or a (scFv)₂. The antibody of the disclosure may be an IgA, IgD, IgE, IgG, or IgM antibody. The IgG antibody may be an IgG1, IgG2, IgG3 or IgG4 antibody, with reduced or no FcR and/or complement system protein binding affinity.

The V_{H} and V_{L} sequences (or CDR sequences) of other antibodies which bind to the Ang2 protein can be “mixed and matched” with the V_{H} and V_{L} sequences (or CDR sequences) of the antibody of the present disclosure. Preferably, when V_{H} and V_{L} chains (or the CDRs within such chains) are mixed and matched, a V_{H} sequence from a particular V_{H}/V_{L} pairing is replaced with a structurally similar V_{H} sequence. Likewise, preferably a V_{L} sequence from a particular V_{H}/V_{L} pairing is replaced with a structurally similar V_{L} sequence.

Modifications can be introduced into an antibody or antigen-binding portion thereof of the disclosure by standard techniques known in the art, such as site-directed mutagenesis and PCR-mediated mutagenesis. One or more amino acid residues within the CDR regions of an antibody or an antigen-binding portion thereof of the disclosure can be replaced with other amino acid residues from the same side chain family and the altered antibody can be tested for retained function (i.e., the functions set forth above) using the functional assays described herein.

Antibodies of the disclosure can be prepared using an antibody having one or more of the V_{H}/V_{L} sequences of the antibody or antigen-binding portion thereof of the present disclosure as starting material to engineer a modified antibody. An antibody can be engineered by modifying one or more residues within one or both variable regions (i.e., V_{H} and/or V_{L}), for example within one or more CDR regions and/or within one or more framework regions. Additionally or alternatively, an antibody can be engineered by modifying residues within the constant region(s), for example to alter the effector function(s) of the antibody.

In certain embodiments, CDR grafting can be used to engineer variable regions of antibodies. Antibodies interact with target antigens predominantly through amino acid residues that are located in the six heavy and light chain complementarity determining regions (CDRs). For this reason, the amino acid sequences within CDRs are more diverse between individual antibodies than sequences outside of CDRs. Because CDR sequences are responsible for most antibody-antigen interactions, it is possible to express recombinant antibodies that mimic the properties of specific naturally occurring antibodies by constructing expression vectors that include CDR sequences from the specific naturally occurring antibody grafted onto framework sequences from a different antibody with different properties. See, e.g., Riechmann et al. (1998) Nature 332:323-327; Jones et al. (1986) Nature 321:522-525; Queen et al. (1989) Proc. Natl. Acad. See also U.S.A. 86:10029-10033; U.S. Pat. Nos. 5,225,539; 5,530,101; 5,585,089; 5,693,762 and 6,180,370.

Accordingly, another embodiment of the disclosure pertains to an isolated monoclonal antibody, or antigen binding portion thereof, which may comprise a heavy chain variable region which may comprise CDR1, CDR2, and CDR3 sequences which may comprise the sequences of the present disclosure, as described above, and/or a light chain variable region which may comprise CDR1, CDR2, and CDR3 sequences which may comprise the sequences of the present disclosure, as described above. While these antibodies contain the V_{H} and V_{L} CDR sequences of the monoclonal antibody of the present disclosure, they can contain different framework sequences.

Framework modification may be used to remove T cell epitopes to thereby reduce the potential immunogenicity of the antibody. This approach is also referred to as “deimmunization” and is described in further detail in U.S. Patent Publication No. 20030153043.

Another type of variable region modification is to mutate amino acid residues within the V_{H} and/or V_{L} CDR1, CDR2 and/or CDR3 regions to thereby improve one or more binding properties (e.g., affinity) of the antibody of interest. Site-directed mutagenesis or PCR-mediated mutagenesis can be performed to introduce the mutation(s) and the effect on antibody binding, or other functional property of interest, can be evaluated in in vitro or in vivo assays as known in the art. Preferably conservative modifications (as known in the art) are introduced. The mutations can be amino acid substitutions, additions or deletions, but are preferably substitutions. Moreover, typically no more than one, two, three, four or five residues within a CDR region are altered.

In addition, or as an alternative to modifications made within the framework or CDR regions, antibodies of the disclosure can be engineered to include modifications within the Fc region, typically to alter one or more functional properties of the antibody, such as serum half-life, complement fixation, Fc receptor binding, and/or antigen-dependent cellular cytotoxicity. Furthermore, an antibody of the disclosure can be chemically modified (e.g., one or more chemical moieties can be attached to the antibody) or be modified to alter its glycosylation, again to alter one or more functional properties of the antibody.

In one embodiment, the hinge region of C_{H1} is modified in such that the number of cysteine residues in the hinge region is altered, e.g., increased or decreased. This approach is described further in U.S. Pat. No. 5,677,425. The number of cysteine residues in the hinge region of C_{H1} is altered to, for example, facilitate assembly of the light and heavy chains or to increase or decrease the stability of the antibody.

In another embodiment, the Fc hinge region of an antibody is mutated to decrease the biological half-life of the antibody. More specifically, one or more amino acid mutations are introduced into the C_{H2}-C_{H3} domain interface region of the Fc-hinge fragment such that the antibody has impaired Staphylococcyl protein A (SpA) binding relative to native Fc-hinge domain SpA binding. This approach is described in further detail in U.S. Pat. No. 6,165,745.

In still another embodiment, the glycosylation of an antibody is modified. For example, a glycosylated antibody can be made (i.e., the antibody lacks glycosylation). Glycosylation can be altered to, for example, increase the affinity of the antibody for antigen. Such carbohydrate modifications can be accomplished by, for example, altering one or more sites of glycosylation within the antibody sequence. For example, one or more amino acid substitutions can be made that result in elimination of one or more variable region framework glycosylation sites to thereby eliminate glycosylation at that site. Such aglycosylation may increase the affinity of the antibody for antigen. See, e.g., U.S. Pat. Nos. 5,714,350 and 6,350,861.

Additionally or alternatively, an antibody can be made that has an altered type of glycosylation, such as a hypofucosylated antibody having reduced amounts of fucosyl residues or an antibody having increased bisecting GlcNac structures. Such carbohydrate modifications can be accomplished by, for example, expressing the antibody in a host cell with altered glycosylation machinery. Cells with altered glycosylation machinery have been described in the art and can be used as host cells in which to express recombinant antibodies of the disclosure to thereby produce an antibody with altered glycosylation. For example, the cell lines Ms704, Ms705, and Ms709 lack the fucosyltransferase gene, FUT8 (α (1,6)-fucosyltransferase), such that antibodies expressed in the Ms704, Ms705, and Ms709 cell lines lack fucose on their carbohydrates. The Ms704, Ms705, and Ms709 FUT8−/− cell lines were created by the targeted disruption of the FUT8 gene in CHO/DG44 cells using two replacement vectors (see U.S. Patent Publication No. 20040110704 and Yamane-Ohnuki et al. (2004) Biotechnol Bioeng 87:614-22). As another example, EP 1,176,195 describes a cell line with a functionally disrupted FUT8 gene, which encodes a fucosyl transferase, such that antibodies expressed in such a cell line exhibit hypofucosylation by reducing or eliminating the α-1, 6 bond-related enzyme. EP 1,176,195 also describes cell lines which have a low enzyme activity for adding fucose to the N-acetylglucosamine that binds to the Fc region of the antibody or does not have the enzyme activity, for example the rat myeloma cell line YB2/0 (ATCC CRL 1662). PCT Publication WO 03/035835 describes a variant CHO cell line, Lec13 cells, with reduced ability to attach fucose to Asn(297)-linked carbohydrates, also resulting in hypofucosylation of antibodies expressed in that host cell (see also Shields et al. (2002) J. Biol. Chem. 277:26733-26740). Antibodies with a modified glycosylation profile can also be produced in chicken eggs, as described in PCT Publication WO 06/089231. Alternatively, antibodies with a modified glycosylation profile can be produced in plant cells, such as Lemna. Methods for production of antibodies in a plant system are disclosed in the U.S. patent application corresponding to Alston & Bird LLP attorney docket No. 040989/314911, filed on Aug. 11, 2006. PCT Publication WO 99/54342 describes cell lines engineered to express glycoprotein-modifying glycosyl transferases (e.g., 0(1,4)-N-acetylglucosaminyltransferase III (GnTIII)) such that antibodies expressed in the engineered cell lines exhibit increased bisecting GlcNac structures which results in increased ADCC activity of the antibodies (see also Umana et al. (1999) Nat. Biotech. 17:176-180). Alternatively, the fucose residues of the antibody can be cleaved off using a fucosidase enzyme; e.g., the fucosidase α-L-fucosidase removes fucosyl residues from antibodies (Tarentino et al. (1975) Biochem. 14:5516-23).

Another modification of the antibodies herein that is contemplated by this disclosure is pegylation. An antibody can be pegylated to, for example, increase the biological (e.g., serum) half-life of the antibody. To pegylate an antibody, the antibody, or fragment thereof, typically is reacted with polyethylene glycol (PEG), such as a reactive ester or aldehyde derivative of PEG, under conditions in which one or more PEG groups become attached to the antibody or antibody fragment. Preferably, the pegylation is carried out via an acylation reaction or an alkylation reaction with a reactive PEG molecule (or an analogous reactive water-soluble polymer). As used herein, the term “polyethylene glycol” is intended to encompass any of the forms of PEG that have been used to derivatize other proteins, such as mono (C₁-C₁₀) alkoxy- or aryloxy-polyethylene glycol or polyethylene glycol-maleimide. In certain embodiments, the antibody to be pegylated is an aglycosylated antibody. Methods for pegylating proteins are known in the art and can be applied to the antibodies of the disclosure. See, e.g., EPO 154 316 and EP0401384.

Antibodies of the disclosure can be characterized by their various physical properties, to detect and/or differentiate different classes thereof.

For example, antibodies can contain one or more glycosylation sites in either the light or heavy chain variable region. Such glycosylation sites may result in increased immunogenicity of the antibody or an alteration of the pK of the antibody due to altered antigen binding (Marshall et al (1972) Annu Rev Biochem 41:673-702; Gala and Morrison (2004) J Immunol 172:5489-94; Wallick et al (1988) J Exp Med 168:1099-109; Spiro (2002) Glycobiology 12:43R-56R; Parekh et al (1985) Nature 316:452-7; Mimura et al. (2000) Mol Immunol 37:697-706). Glycosylation has been known to occur at motifs containing an N-X-S/T sequence. In some instances, it is preferred to have an antibody that does not contain variable region glycosylation. This can be achieved either by selecting antibodies that do not contain the glycosylation motif in the variable region or by mutating residues within the glycosylation region.

In a preferred embodiment, the antibodies do not contain asparagine isomerism sites. The deamidation of asparagine may occur on N-G or D-G sequences and result in the creation of an isoaspartic acid residue that introduces a kink into the polypeptide chain and decreases its stability (isoaspartic acid effect).

Each antibody will have a unique isoelectric point (pI), which generally falls in the pH range between 6 and 9.5. The pI for an IgG1 antibody typically falls within the pH range of 7-9.5 and the pI for an IgG4 antibody typically falls within the pH range of 6-8. There is speculation that antibodies with a pI outside the normal range may have some unfolding and instability under in vivo conditions. Thus, it is preferred to have an antibody that contains a pI value that falls in the normal range. This can be achieved either by selecting antibodies with a pI in the normal range or by mutating charged surface residues.

In another aspect, the disclosure provides nucleic acid molecules that encode heavy and/or light chain variable regions, or CDRs, of the antibodies of the disclosure. The nucleic acids can be present in whole cells, in a cell lysate, or in a partially purified or substantially pure form. A nucleic acid of the disclosure can be, e.g., DNA or RNA and may or may not contain intronic sequences.

Nucleic acids of the disclosure can be obtained using standard molecular biology techniques. For antibodies expressed by hybridomas, cDNAs encoding the light and heavy chains of the antibody made by the hybridoma can be obtained by standard PCR amplification or cDNA cloning techniques. For antibodies obtained from an immunoglobulin gene library (e.g., using phage display techniques), a nucleic acid encoding such antibodies can be recovered from the gene library.

Preferred nucleic acids molecules of the disclosure include those encoding the V_{H} and V_{L} sequences of the antibody or the CDRs. Once DNA fragments encoding V_{H} and V_{L} segments are obtained, these DNA fragments can be further manipulated by standard recombinant DNA techniques, for example to convert the variable region genes to full-length antibody chain genes, to Fab fragment genes or to a scFv gene. In these manipulations, a V_{L}- or V_{H}-encoding DNA fragment is operatively linked to another DNA fragment encoding another protein, such as an antibody constant region or a flexible linker. The term “operatively linked”, as used in this context, is intended to mean that the two DNA fragments are joined such that the amino acid sequences encoded by the two DNA fragments remain in-frame.

The isolated DNA encoding the V_{H} region can be converted to a full-length heavy chain gene by operatively linking the V_{H}-encoding DNA to another DNA molecule encoding heavy chain constant regions (C_{H1}, C_{H2} and C_{H3}). The sequences of mouse/human heavy chain constant region genes are known in the art and DNA fragments encompassing these regions can be obtained by standard PCR amplification. The heavy chain constant region can be an IgG1, IgG2, IgG3, IgG4, IgA, IgE, IgM or IgD constant region, but most preferably may be an IgG1 constant region in the present disclosure. For a Fab fragment heavy chain gene, the V_{H}-encoding DNA can be operatively linked to another DNA molecule encoding only the heavy chain C_{H1} constant region.

The isolated DNA encoding the V_{L} region can be converted to a full-length light chain gene (as well as a Fab light chain gene) by operatively linking the V_{L}-encoding DNA to another DNA molecule encoding the light chain constant region, C_{L}. The sequences of mouse/human light chain constant region genes are known in the art and DNA fragments encompassing these regions can be obtained by standard PCR amplification. In preferred embodiments, the light chain constant region can be a kappa or lambda constant region.

To create a scFv gene, the V_{H}- and V_{L}-encoding DNA fragments are operatively linked to another fragment encoding a flexible linker, such that the V_{H} and V_{L} sequences can be expressed as a contiguous single-chain protein, with the V_{L} and V_{H} regions joined by the flexible linker (see e.g., Bird et al. (1988) Science 242:423-426; Huston et al. (1988) Proc. Natl. Acad. Sci. USA 85:5879-5883; McCafferty et al., (1990) Nature 348:552-554).

The monoclonal antibodies of the disclosure may be isolated from phage display libraries expressing variable domains or CDRs of a desired species. Screening of phage libraries can be accomplished by various techniques known in the art. For example, a human B-cell antibody library in scFv format or a human naive Fab library may be screened for antibodies binding to the Ang2 protein by solution panning with colorimetric plates coated with Ang2 protein over several rounds of selection with increasing stringency. Isolates may be first expressed as scFv or Fab and screened for binding to the receptor binding domain by ELISA, and the selected isolates may then be cloned and expressed as IgG, reanalyzed for binding to Ang2 protein by ELISA and/or SPR and for functional activity in neutralization assays, and transfected in a CHO mammalian cell line for expression of the full IgG antibodies.

The monoclonal antibodies of the disclosure may be also prepared using hybridoma methods known to one of skill in the art. For example, using a hybridoma method, a mouse, rat, rabbit, hamster, or other appropriate host animal, is immunized as described above. In some embodiments, lymphocytes are immunized in vitro. In some embodiments, the immunizing antigen is an Ang2 protein or a fragment thereof. Following immunization, lymphocytes may be isolated and fused with a suitable myeloma cell line using, for example, polyethylene glycol. The hybridoma cells may be selected using specialized media as known in the art and unfused lymphocytes and myeloma cells do not survive the selection process. Hybridomas that produce monoclonal antibodies directed to a chosen antigen can be identified by a variety of methods including, but not limited to, immunoprecipitation, immunoblotting, and in vitro binding assays (e.g., flow cytometry, FACS, ELISA, SPR (e.g., Biacore), and radioimmunoassay). Once hybridoma cells that produce antibodies of the desired specificity, affinity, and/or activity are identified, the clones may be subcloned by limiting dilution or other techniques. The hybridomas can be propagated either in in vitro culture using standard methods or in vivo as ascites tumors in an animal. The monoclonal antibodies can be purified from the culture medium or ascites fluid according to standard methods in the art including, but not limited to, affinity chromatography, ion-exchange chromatography, gel electrophoresis, and dialysis.

Antibodies of the disclosure also can be produced in a host cell transfectoma using, for example, a combination of recombinant DNA techniques and gene transfection methods as is well known in the art (e.g., Morrison, S. (1985) Science 229:1202). In one embodiment, DNA encoding partial or full-length light and heavy chains obtained by standard molecular biology techniques is inserted into one or more expression vectors such that the genes are operatively linked to transcriptional and translational regulatory sequences. In this context, the term “operatively linked” is intended to mean that an antibody gene is ligated into a vector such that transcriptional and translational control sequences within the vector serve their intended function of regulating the transcription and translation of the antibody gene.

The term “regulatory sequence” is intended to include promoters, enhancers and other expression control elements (e.g., polyadenylation signals) that control the transcription or translation of the antibody chain genes. Such regulatory sequences are described, e.g., in Goeddel (Gene Expression Technology. Methods in Enzymology 185, Academic Press, San Diego, Calif. (1990)). Preferred regulatory sequences for mammalian host cell expression include viral elements that direct high levels of protein expression in mammalian cells, such as promoters and/or enhancers derived from cytomegalovirus (CMV), Simian Virus 40 (SV40), adenovirus, e.g., the adenovirus major late promoter (AdMLP) and polyoma. Alternatively, non-viral regulatory sequences can be used, such as the ubiquitin promoter or β-globin promoter. Still further, regulatory elements composed of sequences from different sources, such as the SRα promoter system, which contains sequences from the SV40 early promoter and the long terminal repeat of human T cell leukemia virus type 1 (Takebe et al. (1988) Mol. Cell. Biol. 8:466-472). The expression vector and expression control sequences are chosen to be compatible with the expression host cell used.

The antibody light chain gene and the antibody heavy chain gene can be inserted into the same or separate expression vectors. In preferred embodiments, the variable regions are used to create full-length antibody genes of any antibody isotype by inserting them into expression vectors already encoding heavy chain constant and light chain constant regions of the desired isotype such that the V_{H} segment is operatively linked to the C_{H} segment(s) within the vector and the V_{L} segment is operatively linked to the C_{L} segment within the vector. Additionally or alternatively, the recombinant expression vector can encode a signal peptide that facilitates secretion of the antibody chain from a host cell. The antibody chain gene can be cloned into the vector such that the signal peptide is linked in-frame to the amino terminus of the antibody chain gene. The signal peptide can be an immunoglobulin signal peptide or a heterologous signal peptide (i.e., a signal peptide from a non-immunoglobulin protein).

In addition to the antibody chain genes and regulatory sequences, the recombinant expression vectors of the disclosure can carry additional sequences, such as sequences that regulate replication of the vector in host cells (e.g., origins of replication) and selectable marker genes. The selectable marker gene facilitates selection of host cells into which the vector has been introduced (see, e.g., U.S. Pat. Nos. 4,399,216; 4,634,665 and 5,179,017). For example, typically the selectable marker gene confers resistance to drugs, such as G418, hygromycin or methotrexate, on a host cell into which the vector has been introduced. Preferred selectable marker genes include the dihydrofolate reductase (DHFR) gene (for use in dhfr-host cells with methotrexate selection/amplification) and the neo gene (for G418 selection).

For expression of the light and heavy chains, the expression vector(s) encoding the heavy and light chains is transfected into a host cell by standard techniques. The term “transfection” are intended to encompass a wide variety of techniques commonly used for the introduction of exogenous DNA into a prokaryotic or eukaryotic host cell, e.g., electroporation, calcium-phosphate precipitation, DEAE-dextran transfection and the like. Although it is theoretically possible to express the antibodies of the disclosure in either prokaryotic or eukaryotic host cells, expression of antibodies in eukaryotic cells, and most preferably mammalian host cells, is the most preferred because such eukaryotic cells, and in particular mammalian cells, are more likely than prokaryotic cells to assemble and secrete a properly folded and immunologically active antibody.

Preferred mammalian host cells for expressing the recombinant antibodies of the disclosure include Chinese Hamster Ovary (CHO cells) (including dhfr-CHO cells, described in Urlaub and Chasin, (1980) Proc. Natl. Acad. Sci. USA 77:4216-4220, used with a DHFR selectable marker, e.g., as described in R. J. Kaufman and P. A. Sharp (1982) J. Mol. Biol. 159:601-621), NSO myeloma cells, COS cells and SP2 cells. In particular for use with NSO myeloma cells, another preferred expression system is the GS gene expression system disclosed in WO 87/04462, WO 89/01036 and EP 338,841. When recombinant expression vectors encoding antibody genes are introduced into mammalian host cells, the antibodies are produced by culturing the host cells for a period of time sufficient to allow for expression of the antibody in the host cells or, more preferably, secretion of the antibody into the culture medium in which the host cells are grown. Antibodies can be recovered from the culture medium using standard protein purification methods.

In another aspect, the present disclosure features bispecific molecules which may comprise one or more antibodies of the disclosure linked to at least one other functional molecule, e.g., another peptide or protein (e.g., another antibody or ligand for a receptor) to generate a bispecific molecule that binds to at least two different binding sites or target molecules. For example, the bispecific antibody of the disclosure may be designed to bind two epitopes in the Ang2 protein, or bind one epitope in the Ang2 protein and another protein such as VEGF. The term “bispecific molecule” herein includes molecules that have three or more specificities.

Bispecific molecules can come in many different formats and sizes. At one end of the size spectrum, a bispecific molecule retains the traditional antibody format, except that, instead of having two binding arms of identical specificity, it has two binding arms each having a different specificity. At the other extreme are bispecific molecules consisting of two single-chain antibody fragments (scFv's) linked by a peptide chain, a so-called Bs(scFv) 2 construct. Intermediate-sized bispecific molecules include two different F(ab) fragments linked by a peptidyl linker. Bispecific molecules of these and other formats can be prepared by genetic engineering, somatic hybridization, or chemical methods. See, e.g., Kufer et al, cited supra; Cao and Suresh, Bioconjugate Chemistry, 9 (6), 635-644 (1998); and van Spriel et al., Immunology Today, 21 (8), 391-397 (2000), and the references cited therein.

The composition, e.g., a pharmaceutical composition, of the disclosure may comprise any number of excipients. Excipients that can be used include carriers, surface active agents, thickening or emulsifying agents, solid binders, dispersion or suspension aids, solubilizers, colorants, flavoring agents, coatings, disintegrating agents, lubricants, sweeteners, preservatives, isotonic agents, and combinations thereof. The selection and use of suitable excipients is taught in Gennaro, ed., Remington: The Science and Practice of Pharmacy, 20th Ed. (Lippincott Williams & Wilkins 2003), the disclosure of which is incorporated herein by reference.

Preferably, the pharmaceutical composition is suitable for intravenous, intramuscular, subcutaneous, parenteral, spinal or epidermal administration (e.g., by injection or infusion). The phrase “parenteral administration” as used herein means modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal, epidural and intrasternal injection and infusion. Alternatively, an antibody of the disclosure can be administered via a non-parenteral route, such as a topical, epidermal or mucosal route of administration, e.g., intranasally, orally, vaginally, rectally, sublingually or topically.

Pharmaceutical compositions can be in the form of sterile aqueous solutions or dispersions. They can also be formulated in a microemulsion, liposome, or other ordered structure suitable to high drug concentration.

The amount of active ingredient which can be combined with a carrier material to produce a single dosage form will vary depending upon the subject being treated and the particular mode of administration and will generally be that amount of the composition which produces a therapeutic effect. Generally, out of one hundred percent, this amount will range from about 0.01% to about ninety-nine percent of active ingredient, preferably from about 0.1% to about 70%, most preferably from about 1% to about 30% of active ingredient in combination with a pharmaceutically acceptable carrier.

For administration of the composition, the antibody dose may range from about 0.0001 to 100 mg/kg, and more usually 0.01 to 5 mg/kg, of the host body weight. For example, dosages can be 0.3 mg/kg body weight, 1 mg/kg body weight, 3 mg/kg body weight, 5 mg/kg body weight or 10 mg/kg body weight or within the range of 1-10 mg/kg of the active ingredient. In certain embodiments, the antibody dose may be 0.3 to 30 mg/kg, such as 0.3 mg/kg, 1 mg/kg, 3 mg/kg, 10 mg/kg, and 30 mg/kg. An exemplary treatment regime entails administration once per week, once every two weeks, once every three weeks, once every four weeks, once a month, once every 3 months or once every three to 6 months. Preferred dosage regimens for an anti-Ang2 antibody of the disclosure include 1 mg/kg body weight or 3 mg/kg body weight via intravenous administration, with the antibody being given using one of the following dosing schedules: (i) every four weeks for six dosages, then every three months; (ii) every three weeks; (iii) 3 mg/kg body weight once followed by 1 mg/kg body weight every three weeks. In some methods, dosage is adjusted to achieve a plasma antibody concentration of about 1-1000 μg/ml and in some methods about 25-300 μg/ml.

Dosage regimens are adjusted to provide the optimum desired response (e.g., a therapeutic response). For example, a single dose can be administered, several divided doses can be administered over time or the dose can be proportionally reduced or increased as indicated by the exigencies of the therapeutic situation. It is especially advantageous to formulate parenteral compositions in dosage unit form for ease of administration and uniformity of dosage. Dosage unit form as used herein refers to physically discrete units suited as unitary dosages for the subjects to be treated; each unit contains a predetermined quantity of active ingredient calculated to produce the desired therapeutic effect in association with the required pharmaceutical carrier. Alternatively, antibody can be administered as a sustained release formulation, in which case less frequent administration is required.

In certain embodiments, the compositions of the disclosure can be formulated to ensure proper distribution in vivo. For example, to ensure that the therapeutic antibody of the disclosure cross the blood-brain barrier, they can be formulated in liposomes, which may additionally comprise targeting moieties to enhance selective transport to specific cells or organs. See, e.g. U.S. Pat. Nos. 4,522,811; 5,374,548; 5,416,016; and 5,399,331; V. V. Ranade (1989) J. Clin. Pharmacol. 29:685; Umezawa et al., (1988) Biochem. Biophys. Res. Commun. 153:1038; Bloeman et al. (1995) FEBS Lett. 357:140; M. Owais et al. (1995) Antimicrob. Agents Chemother. 39:180; Briscoe et al. (1995) Am. J. Physiol. 1233:134; Schreier et al. (1994) J. Biol. Chem. 269:9090; Keinanen and Laukkanen (1994) FEBS Lett. 346:123; and Killion and Fidler (1994) Immunomethods 4:273.

The compositions of the present disclosure have numerous in vitro and in vivo utilities involving, for example, detection of Ang2 proteins in vitro, and treatment of Ang2 related diseases. The compositions of the disclosure may be administered to human subjects, e.g., in vivo, to inhibit Ang2 binding to Tie2 or integrins, and/or to activate Tie2 signaling, enabling vascular normalization and/or inhibit unrequired angiogenesis.

In certain embodiments, the compositions of the disclosure may be used to stabilize the vasculature or reduce vascular inflammation. In certain embodiments, the compositions of the disclosure may be used to treat or alleviate cancers, such as ovarian cancer, lung cancer (e.g., Lewis lung carcinoma, small cell lung cancer, and non-small cell lung cancer), mammary cancer, cervical cancer, and kaposi's sarcoma, whether original or metastatic, inflammatory diseases, such as epsis, macular edema, diabetic retinopathy and age-related macular degeneration (e.g., neovascular age-related macular degeneration), and infectious diseases, such as pneumonias caused by infection of bacteria, viruses or mycoplasma.

The compositions of the disclosure comprising comprise one or more antibodies, one or more bispecific molecules, or one or more expression vectors of the disclosure of the present disclosure may be administered in a combination therapy with, for example, another agent for treating the Ang2 related diseases, such as an anti-tumor agent, and anti-inflammatory agent, or an anti-infectious agent.

The combination of therapeutic agents discussed herein can be administered concurrently as a single composition in a pharmaceutically acceptable carrier, or concurrently as separate compositions with each agent in a pharmaceutically acceptable carrier. In another embodiment, the combination of therapeutic agents can be administered sequentially.

Furthermore, if more than one dose of the combination therapy is administered sequentially, the order of the sequential administration can be reversed or kept in the same order at each time point of administration, sequential administrations can be combined with concurrent administrations, or any combination thereof.

Although the present invention and its advantages have been described in detail, it should be understood that various changes, substitutions and alterations can be made herein without departing from the spirit and scope of the invention as defined in the appended claims.

The present invention will be further illustrated in the following Examples which are given for illustration purposes only and are not intended to limit the invention in any way.

### Examples

### Example 1: Generation of Anti-Ang2 Monoclonal Antibodies (mAbs)

### Immunization

Balb/c and A/J mice, as well as Wista rats, were immunized with human Ang2 proteins (R&D systems, catalog #623-AN/CF) under current animal welfare regulations. For immunization, the antigen was administrated in PBS solution or formulated as an emulsion with CFA (Complete Freund's adjuvant; for primary immunization) or IFA (incomplete Freund's adjuvant; for boost immunizations). Each animal received 3-5 doses. Seven days after each administration, 20 μL of blood samples were collected from the animals to monitor the anti-sera titer in an ELISA-based assay with immobilized Ang2 protein as a control, till the fusion criteria were met.

### Selections of Anti-Ang2 mAb Secreting Hybridoma

Three days after the last immunization, splenocytes from selected mice were extracted and fused with sp2/0 cells following standard hybridoma generation protocol in sterile environment. The fused cells were cultured in 1×HAT (hypoxanthine-aminopterin-thymidine) containing DMEM media supplemented with 10% FBS for 7 days. The contents in the supernatant were analyzed for their ability of binding to Ang2 proteins by ELISA. Their specificity to Ang2 target was confirmed with Ang1 protein in ELISA counter screening. The positive parental clones were subcloned by limited dilution and cultured in 1×HT (hypoxanthine-thymidine) containing DMEM media, supplemented with 10% FBS. Cells were cultured for 1 week before a new round of screening for positive monoclones.

The positive clones were further tested in a competition ELISA for their blocking activities on Ang2-Tie2 binding. Briefly, 100 μl of 0.5 μg/ml human Tie2-Fc proteins (Genscript) were pre-coated onto ELISA plates overnight at 4° C. On the next day, wells were incubated with supernatants of hybridoma expressing anti-Ang2 antibodies and 100 ng/ml biotin-labeled Ang2 at room temperature for 2 hours. After washing for five times with PBST, streptavidin-HRP (Genscript) was added into each well to react at room temperature for 1 hour. After washing for five times with PBST, the plates were added with TMB for chromogenic reaction. The ELISA binding OD450 for Ang2-Tie2 binding without antibody addition was set as the baseline. If the addition of one antibody increased the OD450 value compared to the baseline, then this antibody might potentially promote Ang2-Tie2 binding. On the contrary, if the addition of one antibody lowered the OD450 value compared to the baseline, this antibody may potentially block Ang2-Tie2 binding.

ABA (4H10), an anti-Ang2 antibody synthesized according to US patent U.S. Pat. No. 9,902,767B2 with human IgG1/kappa constant regions (SEQ ID NO: 259) that inhibited Ang2-Tie2 binding, and ABTAA (10D6), an anti-Ang2 antibody synthesized according to the same US patent with human IgG4/kappa constant regions that bound Ang2 and promoted Ang2 and Tie2 clustering, were used as controls.

Based on the data collected from the assays, fifty-two hybridomas expressing unique mAbs were selected, including 41E4F2, 41F7H8, 41H8A8, 42A2A6, 42C3A4, 43B2A12, 43C4A11, 43D3A9, 43F1C10, 43F1C10-2, 43G5G3, 45C5A1, 45C9F9, 45C9F9-2, 46F3H4, 46H3H3, 49C3B8, 49E4C2, 49E4F1, 49E9G10, 49G4D6, 4D11H2, 4D8F2, 50B9C10, 50B9C10-2, 50C9C2, 50F11D12, 50H8G3, 54E4A12, 5A7B9, 5F7D7, 5F7D7-2, 76B2E6, 7F10B2, 31E2D4, 41A6A6, 39C1C4, 49B8G4, 27H11B5, 15H10D12, 25C5D6F3, 17F3E8, 82B10C1, 53E8B2, 60C4B9, 73H11G10, 15B4C6, 50H8E5C5, 85E10B9, 55D3F10, 98C7H11 and 33A3E2.

Antibody isotypes were tested (Clonotyping System-HRP, SouthernBiotech) and antibodies were purified with Protein-A magnetic beads, eluted by 0.5M Sodium-citrate solution (pH3.5), neutralized with 0.5M Tris-HCl (pH9.0). The storage buffer was changed into PBS to determine concentration with nanodrops.

### Example 2: Characterization of Anti-Ang2 Mouse Antibodies In Vitro

### Indirect ELISA

The antibodies were tested for their binding activities to Ang2 in an indirect ELISA. Briefly, 100 μl of 0.5 μg/ml human or mouse Ang2 proteins (R&D Systems; 623-AN-01M/CF for human Ang2, 7186-AN-025/CF for mouse Ang2) were pre-coated onto ELISA plates overnight at 4° C. On the next day, the plates were incubated with 100 μ3×serially diluted primary antibodies, with initial concentration at 1 μg/ml. The plates were added with HRP-conjugated goat anti-mouse IgG (H+L) and then TMB for chromogenic reaction. The antibody-antigen binding curves were generated with optical density readings at 450 nm. Raw data was plotted using GraphPad Prism v6.02 software with four parameters, and best-fit values program was used to analyze the EC₅₀.

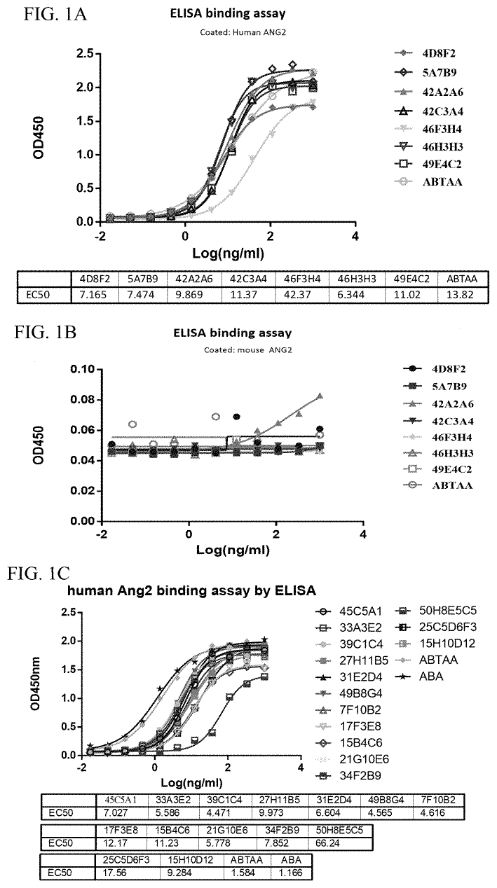

As shown in FIGS. 1A-1C, the antibodies 4D8F2, 5A7B9, 42A2A6, 42C3A4, 46F3H4, 46H3H3 and 49E4C2 bound to human Ang2 proteins (FIG. 1A), most with lower EC₅₀ than ABTAA, but showed undetectable binding activity to mouse Ang2 (FIG. 1B); some other anti-Ang2 antibodies of the disclosure showed binding activities to human Ang2 (FIG. 1C).

### Cell Based Binding Assay

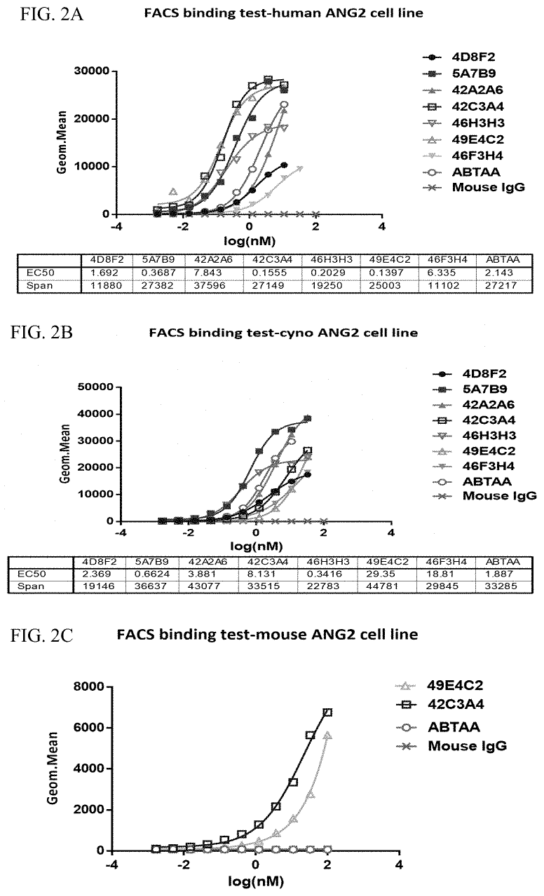

Seven antibodies were further tested for their binding activities to CHO-K1-Ang2 cells by FACS. Briefly, CHO-K1 cells expressing membrane-anchored human, cyno or mouse Ang2 proteins (Custom service by Genscript) were harvested and incubated with 100 μl serially diluted anti-Ang2 mAbs, followed by fluorophore (iFluor 647)-labeled goat anti mouse IgG (H+L) secondary antibodies. The samples were then analyzed with flow cytometry. The binding curves were shown in FIG. 2. Raw data was plotted with GraphPad Prism v6.02 software to analyze the EC₅₀.

As shown in FIGS. 2A-2C, all the 7 antibodies bound to human (FIG. 2A) and cyno (FIG. 2B) Ang2 proteins, some with higher Emax (maximum binding) and/or lower EC₅₀ than ABTAA. Interestingly, two antibodies 49E4C2 and 42C3A4 bound to mouse Ang2 proteins (FIG. 2C) in FACS.

### Cell Based Phos-AKT Assay

The antibodies of the disclosure were tested for their effects on Tie2 signaling activation in a cell based phos-AKT bioassay.

HUVEC cells (ATCC) were cultured in a 10 cm dish with ECM complete medium (Sciencell) at 37° C. and 5% CO₂. When HUVEC cells reached 80%-90% confluency, 2 mL Accutase solution (Gibco) were used to digest cells, and cells were then resuspended in ECM complete culture medium to achieve a concentration of 1×10⁶ cells/mL. The cell suspension of 50 μL was added to each well in 96-well flat-bottom plates and incubated at 37° C. in a 5% CO₂ incubator overnight. After discarding all cell culture medium in the well, 50 μL fresh serum-free media were added and incubated for 16 hours at 37° C. in the 5% CO₂ incubator.

The HUVEC cell containing plates were added with 25 μL serially diluted anti-Ang2 antibodies of the disclosure (5-fold dilution with an initial concentration at 200 μg/mL) and 25 μL of 4 μg/mL Ang2 proteins (R&D Systems; 623-AN-01M/CF). The plates were incubated at 37° C. in the 5% CO₂ incubator for 10 minutes.

PHOSPH-AKT (SER473) kit (Cisbio) was utilized to detect AKT phosphorylation on serine 473. In specific, 50 μL of supplemented lysis buffer was added into each well and incubated for 35 minutes at room temperature with shaking. After that, 16 μL of cell lysate from each well was transferred to detection plates. After added with 4 μL prepared antibody solution (Ciobio) in each well and sealed, the plates were incubated at room temperature for 4 hours. The phosphorylation level of Tie2/AKT was measured by reading the fluorescence emission at 665 nm and 620 nm on PheraStar (a HTRFR plate reader).

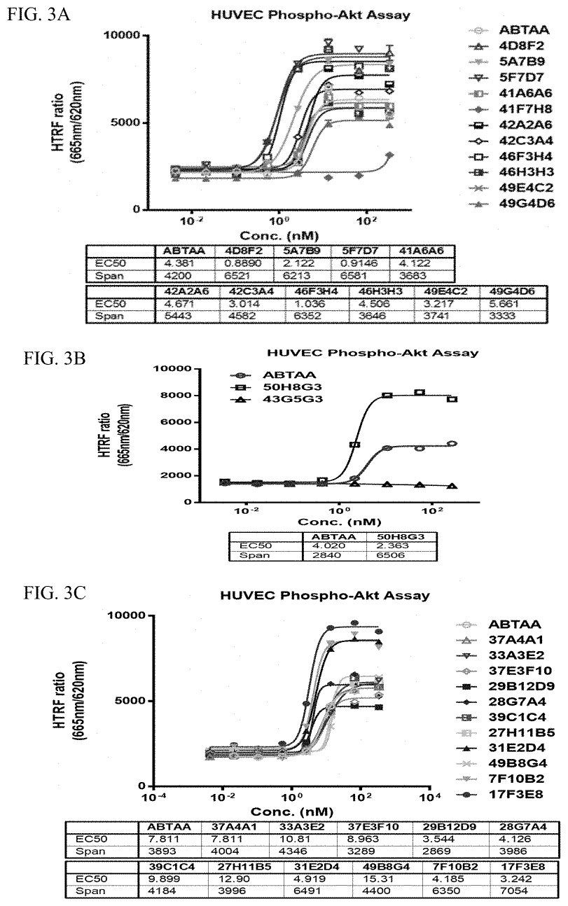

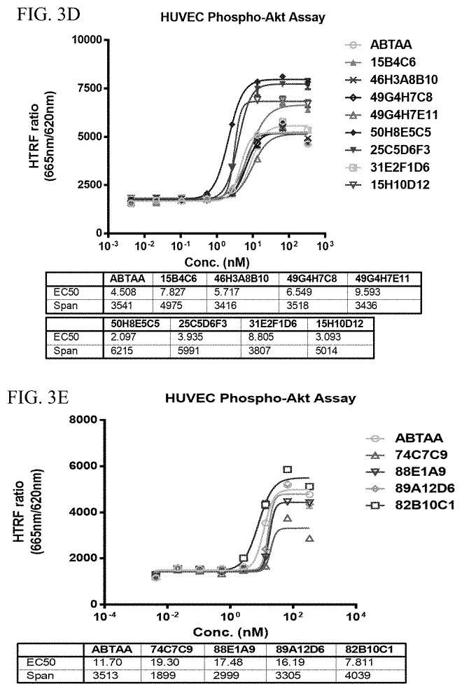

The phos-AKT bioassay results were shown in FIGS. 3A-3E. Compared with ABTAA, several antibodies of the disclosure functioned as better Tie2 agonists, including 4D8F2, 5A7B9, 5F7D7 and 46F3H4 (FIG. 3A), 50H8G3 (FIG. 3B), 17F3E8, 7F10B2 and 31E2D4 (FIG. 3C), 50H8E5C5, 25C5D6F3 and 15H10D12 (FIG. 3D), 82B10C1 (FIG. 3E), with much lower EC₅₀ and higher spans. The remaining antibodies showed lower EC₅₀ or higher span, or comparable agonistic activities.

### Tie2-Ang2 Blockade Assay

The antibodies of the disclosure were tested for blocking activities on Ang2 binding to Tie2, using engineered Tie2 effector cells, which were HT-1080 cells expressing human Tie2 with a luciferase reporter driven by an NFkB response element (NFkB-RE). When these cells are co-cultured with human Ang2 proteins, the Tie2-Ang2 interaction would activate NFkB-RE-mediated luminescence. When anti-Ang2 antibodies that inhibit Ang2-Tie2 binding are added, these cells release inhibitory signals as reflected by decreased NFkB-RE-mediated luminescence.

The Tie2 effector cells were seeded in 6-cm dish, and added and incubated with 2 mL of Accutase solution (Gibco) at 37° C. for 3 minutes. The dishes were added with 3 mL of Tie2 effector cell culture medium (EMEM/10% FBS) and then subjected to centrifugation at 800 rpm for 4 minutes. The cell pellets were gently resuspended in culture medium to achieve a concentration of 5×10⁵ cells/mL. The suspension was then transferred to a sterile reagent reservoir, and the 20 μL aliquots were transferred to 384-well plates and incubated at 37° C. in 5% CO₂ incubator for 20 hours.

The cell containing plates were added with 10 μL serially diluted anti-Ang2 antibodies of the disclosure (3-fold dilution with an initial concentration at 200 μg/mL) and 10 μL of 8 μg/mL Ang2 proteins (R&D Systems; 623-AN-01M/CF). The plates were incubated at 37° C. in the 5% CO₂ incubator for 6 hours. Then, 40 μL of Bio-Glo reagent (Promega) was added into each well. The plates were incubated at room temperature for 5-15 minutes. The luminescence signal was measured by a luminescence plate reader, and the data was analyzed by GraphPad Prism software. Antibody ABA was used as a positive control.

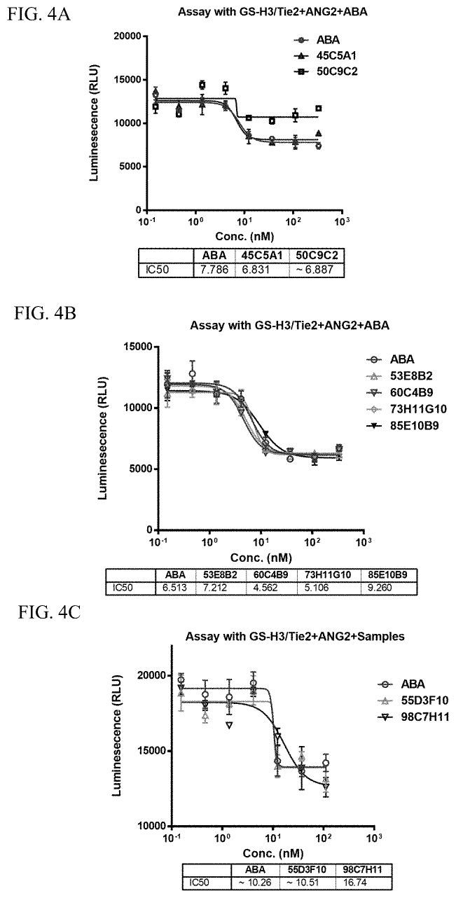

As shown in FIGS. 4A-4C, antibodies 45C5A1 (FIG. 4A), 53E8B2, 60C4B9, 73H11G10, 85E10B9 (FIG. 4B), 55D3F10 and 98C7H11 (FIG. 4C) were capable of blocking Ang2 binding to Tie2.

### Example 3: Chimeric Antibody Production and Characterization

### Construction of Chimeric Antibodies

The heavy chain and light chain variable regions of the mAbs of the disclosures were sequenced, and variable region coding sequences were optimized for human codon biased expression with GenScript online tools. The DNA fragments were synthesized and fused to human IgG4 heavy chain domains (CH1-hinge-CH2-CH3, SEQ ID NO: 257) and kappa light chain constant regions (SEQ ID NO: 258) respectively for transient expression in chimeric formats. The heavy chain and light chain expression constructs were cloned into individual pTT5 based plasmids with signal peptide.

The chimeric antibodies were expressed in CHO-3E7 cells transfected with antibody heavy chain/light pair plasmids using PEImax 40,000 (polysciences). Twenty four hours later, the expression/secretion was boosted with Tryptone N−1 supplement. After 6 days of shaking culture in 37° C. and 5% CO₂, supernatants were collected and antibodies were purified with Protein-A beads as described above. Chimeric antibody products were kept in PBS for analysis.

### Cell Based Phos-AKT Assay

The phos-AKT bioassay was conducted for the chimeric antibodies, following the protocol in Example 2.

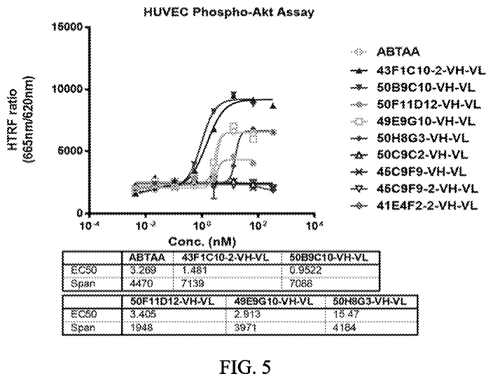

As shown in FIG. 5, chimeric antibodies 43F1C10-2 and 50B9C10 showed much lower EC₅₀ and higher span value than ABTAA, while 49E9G10 showed a bit lower EC₅₀ and span value than ABTAA.

### Tie2-Ang2 Blockade Assay

The Tie2-Ang2 blockade bioassay was performed as described in Example 2.

### Example 4: Antibody Humanization and Characterization

Antibody Humanization and Production

Based on the variable region sequences, the CDRs, HV loops and FRs were analyzed and homology modeling was performed to obtain the modeled structures of the mouse antibodies. The solvent accessible surface area of framework residues were calculated, and framework residues that were buried (i.e. with solvent accessible surface area of <15%) were identified. Three (3) human acceptors for V_{H} and V_{L} were selected that shared the top sequences identical to the mouse counterparts. The CDRs of the mouse antibodies were directed grafted to the human acceptor frameworks. The post translational modifications and chemical degradation in grafted sequences were determined, including deamidation, isomerization oxidation and glycosylation etc. through developability assessment. PTM hotspots like N-glycosylation sites, unusual proline residues, deamidation site, isomerization site, oxidation site and unpaired cysteine residues etc. that may affect the binding activity and manufacturability of the grafted antibody were identified.

DNA sequences encoding the humanized light and heavy chains were synthesized, and antibodies were expressed in CHO-3E7 cells transfected with antibody heavy chain/light pair plasmids using PEImax 40,000 (polysciences). Twenty four hours later, the expression/secretion was boosted with Tryptone N−1 supplement. After 6 days of shaking culture in 37° C. and 5% CO₂, supernatants were collected and the humanized antibodies were purified with Protein-A beads as described above. The humanized antibodies were kept in PBS for analysis.

The humanized heavy chain and light chain of antibodies were combined for antibody production. Humanized 17F3E8 and 5F7D7 light chains share the same protein sequence because they have the same CDRs. Similarly, 31E2D4 and 5A7B9 share the same light chains, and this is also the case for humanized 73H11G10 and 53E8B2 light chains. For each heavy chain and light chain combination, the detailed information is shown in Table 2 and 3.

TABLE 2
humanized antibody production by heavy
chain and light chain pairing

| Antibody name | Heavy chain | Light chain |
|---|---|---|
| 46H3H3-VH1-VL1 | 46H3H3-VH1 | 46H3H3-VL1 |
| | (SEQ ID NO: 260) | (SEQ ID NO: 297) |
| 46H3H3-VH1-VL2 | 46H3H3-VH1 | 46H3H3-VL2 |
| | (SEQ ID NO: 260) | (SEQ ID NO: 298) |
| 46H3H3-VH1-VL3 | 46H3H3-VH1 | 46H3H3-VL3 |
| | (SEQ ID NO: 260) | (SEQ ID NO: 299) |
| 46H3H3-VH2-VL1 | 46H3H3-VH2 | 46H3H3-VL1 |
| | (SEQ ID NO: 261) | (SEQ ID NO: 297) |
| 46H3H3-VH2-VL2 | 46H3H3-VH2 | 46H3H3-VL2 |
| | (SEQ ID NO: 261) | (SEQ ID NO: 298) |
| 46H3H3-VH2-VL3 | 46H3H3-VH2 | 46H3H3-VL3 |
| | (SEQ ID NO: 261) | (SEQ ID NO: 299) |
| 46H3H3-VH3-VL1 | 46H3H3-VH3 | 46H3H3-VL1 |
| | (SEQ ID NO: 262) | (SEQ ID NO: 297) |
| 46H3H3-VH3-VL2 | 46H3H3-VH3 | 46H3H3-VL2 |
| | (SEQ ID NO: 262) | (SEQ ID NO: 298) |
| 46H3H3-VH3-VL3 | 46H3H3-VH3 | 46H3H3-VL3 |
| | (SEQ ID NO: 262) | (SEQ ID NO: 299) |
| 42C3A4-VH1-VL1 | 42C3A4-VH1 | 42C3A4-VL1 |
| | (SEQ ID NO: 263) | (SEQ ID NO: 300) |
| 42C3A4-VH1-VL2 | 42C3A4-VH1 | 42C3A4-VL2 |
| | (SEQ ID NO: 263) | (SEQ ID NO: 301) |
| 42C3A4-VH1-VL3 | 42C3A4-VH1 | 42C3A4-VL3 |
| | (SEQ ID NO: 263) | (SEQ ID NO: 302) |
| 42C3A4-VH2-VL1 | 42C3A4-VH2 | 42C3A4-VL1 |
| | (SEQ ID NO: 264) | (SEQ ID NO: 300) |
| 42C3A4-VH2-VL2 | 42C3A4-VH2 | 42C3A4-VL2 |
| | (SEQ ID NO: 264) | (SEQ ID NO: 301) |
| 42C3A4-VH2-VL3 | 42C3A4-VH2 | 42C3A4-VL3 |
| | (SEQ ID NO: 264) | (SEQ ID NO: 302) |
| 42C3A4-VH3-VL1 | 42C3A4-VH3 | 42C3A4-VL1 |
| | (SEQ ID NO: 265) | (SEQ ID NO: 300) |
| 4D8F2-VH1-VL1 | 4D8F2-VH1 | 4D8F2-VL1 |
| | (SEQ ID NO: 266) | (SEQ ID NO: 303) |
| 4D8F2-VH1.1-VL1 | 4D8F2-VH1.1 | 4D8F2-VL1 |
| | (SEQ ID NO: 267) | (SEQ ID NO: 303) |
| 4D8F2-VH1.2-VL1 | 4D8F2-VH1.2 | 4D8F2-VL1 |
| | (SEQ ID NO: 268) | (SEQ ID NO: 303) |
| 4D8F2-VH1.3-VL1 | 4D8F2-VH1.3 | 4D8F2-VL1 |
| | (SEQ ID NO: 269) | (SEQ ID NO: 303) |
| 4D8F2-VH2-VL2 | 4D8F2-VH2 | 4D8F2-VL2 |
| | (SEQ ID NO: 270) | (SEQ ID NO: 304) |
| 4D8F2-VH3-VL3 | 4D8F2-VH3 | 4D8F2-VL3 |
| | (SEQ ID NO: 271) | (SEQ ID NO: 305) |
| 49E4C2-VH1-VL1 | 49E4C2-VH1 | 49E4C2-VL1 |
| | (SEQ ID NO: 325) | (SEQ ID NO: 324) |
| 42A2A6-VH1-VL1 | 42A2A6-VH1 | 42A2A6-VL1 |
| | (SEQ ID NO: 272) | (SEQ ID NO: 306) |
| 42A2A6-VH1-VL2 | 42A2A6-VH1 | 42A2A6-VL2 |
| | (SEQ ID NO: 272) | (SEQ ID NO: 307) |
| 42A2A6-VH1-VL3 | 42A2A6-VH1 | 42A2A6-VL3 |
| | (SEQ ID NO: 272) | (SEQ ID NO: 308) |
| 42A2A6-VH1-VL4 | 42A2A6-VH1 | 42A2A6-VL4 |
| | (SEQ ID NO: 272) | (SEQ ID NO: 309) |
| 42A2A6-VH2-VL1 | 42A2A6-VH2 | 42A2A6-VL1 |
| | (SEQ ID NO: 273) | (SEQ ID NO: 306) |
| 42A2A6-VH2-VL2 | 42A2A6-VH2 | 42A2A6-VL2 |
| | (SEQ ID NO: 273) | (SEQ ID NO: 307) |
| 42A2A6-VH2-VL3 | 42A2A6-VH2 | 42A2A6-VL3 |
| | (SEQ ID NO: 273) | (SEQ ID NO: 308) |
| 42A2A6-VH2-VL4 | 42A2A6-VH2 | 42A2A6-VL4 |
| | (SEQ ID NO: 273) | (SEQ ID NO: 309) |
| 5A7B9-VH1-VL1 | 5A7B9-VH1 | 5A7B9-VL1 |
| | (SEQ ID NO: 274) | (SEQ ID NO: 310) |
| 5A7B9-VH1-VL2 | 5A7B9-VH1 | 5A7B9-VL2 |
| | (SEQ ID NO: 274) | (SEQ ID NO: 311) |
| 5A7B9-VH2-VL1 | 5A7B9-VH2 | 5A7B9-VL1 |
| | (SEQ ID NO: 275) | (SEQ ID NO: 310) |
| 5A7B9-VH2-VL2 | 5A7B9-VH2 | 5A7B9-VL2 |
| | (SEQ ID NO: 275) | (SEQ ID NO: 311) |
| 5A7B9-VH2-VL3 | 5A7B9-VH2 | 5A7B9-VL3 |
| | (SEQ ID NO: 275) | (SEQ ID NO: 312) |
| 5A7B9-VH3-VL1 | 5A7B9-VH3 | 5A7B9-VL1 |
| | (SEQ ID NO: 276) | (SEQ ID NO: 310) |
| 5A7B9-VH3-VL2 | 5A7B9-VH3 | 5A7B9-VL2 |
| | (SEQ ID NO: 276) | (SEQ ID NO: 311) |
| 5A7B9-VH3-VL3 | 5A7B9-VH3 | 5A7B9-VL3 |
| | (SEQ ID NO: 276) | (SEQ ID NO: 312) |
| 5A7B9-VH4-VL4 | 5A7B9-VH4 | 5A7B9-VL4 |
| | (SEQ ID NO: 277) | (SEQ ID NO: 313) |
| 5A7B9-VH3-VL1.1 | 5A7B9-VH3 | 5A7B9-VL1.1 |
| | (SEQ ID NO: 276) | (SEQ ID NO: 314) |
| 5A7B9-VH3-VL1.2 | 5A7B9-VH3 | 5A7B9-VL1.2 |
| | (SEQ ID NO: 276) | (SEQ ID NO: 315) |
| 5A7B9-VH3-VL1.3 | 5A7B9-VH3 | 5A7B9-VL1.3 |
| | (SEQ ID NO: 276) | (SEQ ID NO: 316) |
| 5A7B9-VH3-VL2.1 | 5A7B9-VH3 | 5A7B9-VL2.1 |
| | (SEQ ID NO: 276) | (SEQ ID NO: 317) |
| 5A7B9-VH3.6-VL1 | 5A7B9-VH3.6 | 5A7B9-VL1 |
| | (SEQ ID NO: 281) | (SEQ ID NO: 310) |
| 5A7B9-VH3.7-VL1 | 5A7B9-VH3.7 | 5A7B9-VL1 |
| | (SEQ ID NO: 282) | (SEQ ID NO: 310) |
| 5A7B9-VH3.1-VL1 | 5A7B9-VH3.1 | 5A7B9-VL1 |
| | (SEQ ID NO: 278) | (SEQ ID NO: 310) |
| 5A7B9-VH3.2-VL1 | 5A7B9-VH3.2 | 5A7B9-VL1 |
| | (SEQ ID NO: 279) | (SEQ ID NO: 310) |
| 5A7B9-VH3.3-VL1 | 5A7B9-VH3.3 | 5A7B9-VL1 |
| | (SEQ ID NO: 280) | (SEQ ID NO: 310) |
| 5A7B9-VH3.3-VL2 | 5A7B9-VH3.3 | 5A7B9-VL2 |
| | (SEQ ID NO: 280) | (SEQ ID NO: 311) |
| 17F3E8-VH1.4-VL2 | 17F3E8-VH1.4 | 5F7D7-VL2 |
| | (SEQ ID NO: 283) | (SEQ ID NO: 319) |
| 17F3E8-VH1.5-VL2 | 17F3E8-VH1.5 | 5F7D7-VL2 |
| | (SEQ ID NO: 284) | (SEQ ID NO: 319) |
| 17F3E8-VH1.4-VL2.1 | 17F3E8-VH1.4 | 5F7D7-VL2.1 |
| | (SEQ ID NO: 283) | (SEQ ID NO: 318) |
| 17F3E8-VH1.5-VL2.1 | 17F3E8-VH1.5 | 5F7D7-VL2.1 |
| | (SEQ ID NO: 284) | (SEQ ID NO: 318) |
| 31E2D4-VH1-VL1 | 31E2D4-VH1 | 5A7B9-VL1 |
| | (SEQ ID NO: 288) | (SEQ ID NO: 310) |
| 31E2D4-VH3-VL1 | 31E2D4-VH3 | 5A7B9-VL1 |
| | (SEQ ID NO: 290) | (SEQ ID NO: 310) |
| 31E2D4-VH4-VL1 | 31E2D4-VH4 | 5A7B9-VL1 |
| | (SEQ ID NO: 291) | (SEQ ID NO: 310) |
| 31E2D4-VH1-VL2 | 31E2D4-VH1 | 5A7B9-VL2 |
| | (SEQ ID NO: 288) | (SEQ ID NO: 311) |
| 31E2D4-VH4-VL2 | 31E2D4-VH4 | 5A7B9-VL2 |
| | (SEQ ID NO: 291) | (SEQ ID NO: 311) |
| 31E2D4-VH1-VL3 | 31E2D4-VH1 | 5A7B9-VL3 |
| | (SEQ ID NO: 288) | (SEQ ID NO: 312) |
| 5F7D7-VH3-VL5 | 5F7D7-VH3 | 5F7D7-VL5 |
| | (SEQ ID NO: 285) | (SEQ ID NO: 320) |
| 5F7D7-VH5-VL5 | 5F7D7-VH5 | 5F7D7-VL5 |
| | (SEQ ID NO: 286) | (SEQ ID NO: 320) |
| 5F7D7-VH6-VL5 | 5F7D7-VH6 | 5F7D7-VL5 |
| | (SEQ ID NO: 287) | (SEQ ID NO: 320) |
| 7F10B2-VH1-VL3 | 7F10B2-VH1 | 7F10B2-VL3 |
| | (SEQ ID NO: 292) | (SEQ ID NO: 323) |
| 7F10B2-VH2-VL1 | 7F10B2-VH2 | 7F10B2-VL1 |
| | (SEQ ID NO: 293) | (SEQ ID NO: 321) |
| 7F10B2-VH2-VL2 | 7F10B2-VH2 | 7F10B2-VL2 |
| | (SEQ ID NO: 293) | (SEQ ID NO: 322) |
| 7F10B2-VH2-VL3 | 7F10B2-VH2 | 7F10B2-VL3 |
| | (SEQ ID NO: 293) | (SEQ ID NO: 323) |
| 7F10B2-VH3-VL1 | 7F10B2-VH3 | 7F10B2-VL1 |
| | (SEQ ID NO: 294) | (SEQ ID NO: 321) |
| 7F10B2-VH3-VL2 | 7F10B2-VH3 | 7F10B2-VL2 |
| | (SEQ ID NO: 294) | (SEQ ID NO: 322) |
| 7F10B2-VH3-VL3 | 7F10B2-VH3 | 7F10B2-VL3 |
| | (SEQ ID NO: 294) | (SEQ ID NO: 323) |
| 7F10B2-VH5-VL1 | 7F10B2-VH5 | 7F10B2-VL1 |
| | (SEQ ID NO: 295) | (SEQ ID NO: 321) |
| 7F10B2-VH6-VL2 | 7F10B2-VH6 | 7F10B2-VL2 |
| | (SEQ ID NO: 296) | (SEQ ID NO: 322) |

TABLE 3
humanized antibody production by heavy
chain and light chain pairing

| Antibody name | Heavy chain | Light chain |
|---|---|---|
| 53E8B2-VH1-VL3 | 53E8B2-VH1 | 53E8B2-VL3 |
| | (SEQ ID NO: 326) | (SEQ ID NO: 336) |
| 53E8B2-VH1-VL6 | 53E8B2-VH1 | 53E8B2-VL6 |
| | (SEQ ID NO: 326) | (SEQ ID NO: 337) |
| 53E8B2-VH2-VL2 | 53E8B2-VH2 | 53E8B2-VL2 |
| | (SEQ ID NO: 327) | (SEQ ID NO: 338) |
| 53E8B2-VH3-VL2 | 53E8B2-VH3 | 53E8B2-VL2 |
| | (SEQ ID NO: 328) | (SEQ ID NO: 338) |
| 85E10B9-VH1-VL6 | 85E10B9-VH1 | 85E10B9-VL6 |
| | (SEQ ID NO: 329) | (SEQ ID NO: 341) |
| 85E10B9-VH3-VL1 | 85E10B9-VH3 | 85E10B9-VL1 |
| | (SEQ ID NO: 330) | (SEQ ID NO: 339) |
| 85E10B9-VH3-VL2 | 85E10B9-VH3 | 85E10B9-VL2 |
| | (SEQ ID NO: 330) | (SEQ ID NO: 340) |
| 53E8B2-VH2-VL6 | 53E8B2-VH2 | 53E8B2-VL6 |
| | (SEQ ID NO: 327) | (SEQ ID NO: 337) |
| 60C4B9-VH1-VL6 | 60C4B9-VH1 | 60C4B9-VL6 |
| | (SEQ ID NO: 331) | (SEQ ID NO: 342) |
| 73H11G10-VH1-VL6 | 73H11G10-VH1 | 73H11G10-VL6 |
| | (SEQ ID NO: 335) | (SEQ ID NO: 337) |
| 98C7H11-VH3-VL2 | 98C7H11-VH3 | 98C7H11-VL2 |
| | (SEQ ID NO: 332) | (SEQ ID NO: 343) |
| 55D3F10-VH1-VL6 | 55D3F10-VH1 | 55D3F10-VL6 |
| | (SEQ ID NO: 333) | (SEQ ID NO: 345) |
| 55D3F10-VH2-VL6 | 55D3F10-VH2 | 55D3F10-VL6 |
| | (SEQ ID NO: 334) | (SEQ ID NO: 345) |
| 55D3F10-VH2-VL2 | 55D3F10-VH2 | 55D3F10-VL2 |
| | (SEQ ID NO: 334) | (SEQ ID NO: 344) |

### Cell Based Phos-AKT Bioassay

The humanized antibodies were tested in the phos-AKT bioassay as described above.

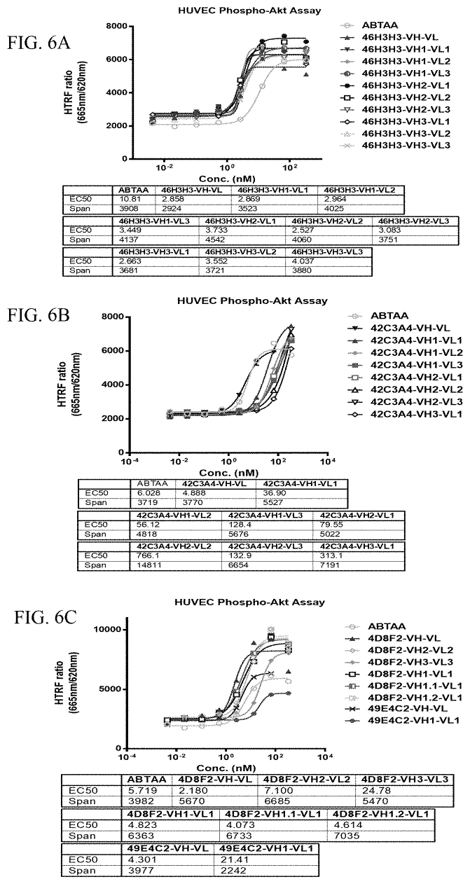

As shown in FIGS. 6A-6C, for 461H31H3 humanization (FIG. 6A), all of humanized 461H31H3 leads showed lower EC₅₀ than ABTAA; for 42C3A4 humanization (FIG. 61B), all of humanized 42C3A4 leads showed higher span value than ABTAA; for 4D8F2 humanization (FIG. 6C), humanized 4D8F2 leads of 4D8F2-VH1-VL1, 4D8F2-VH1.1-VL1 and 4D8F2-VH1.2-VL1 showed lower EC₅₀ and higher span value than ABTAA.

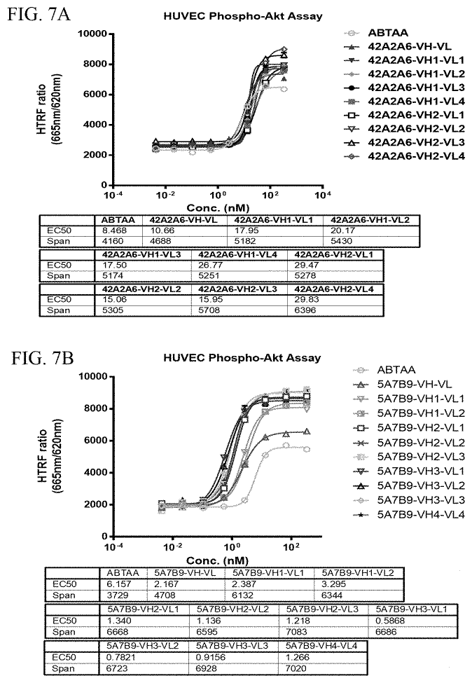

As shown in FIGS. 7A-71B, for 42A2A6 humanization (FIG. 7A), all of humanized 42A2A6 leads showed higher span value than ABTAA; for 5A7B39 humanization (FIG. 71B), all of humanized 5A7B39 leads showed much lower EC₅₀ and higher span value than ABTAA. In particular, humanized 5A7B39 lead of 5A7B39-VH3-VL1 showed 10-fold lower EC₅₀ than ABTAA.

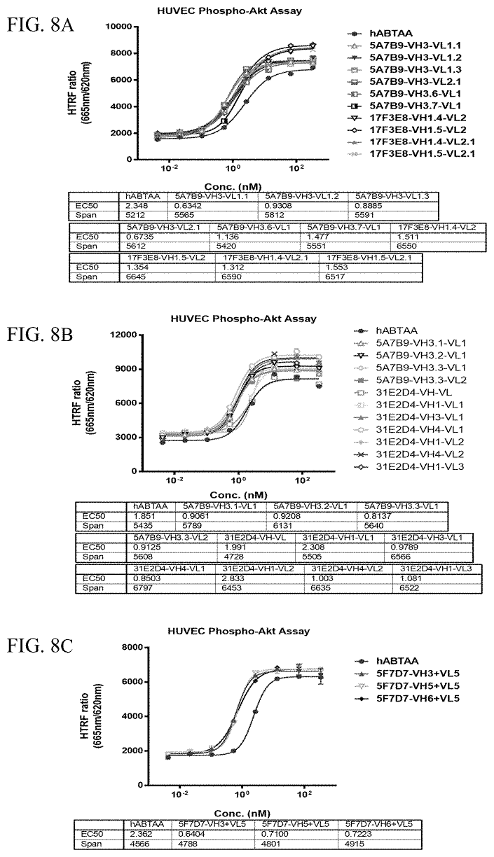

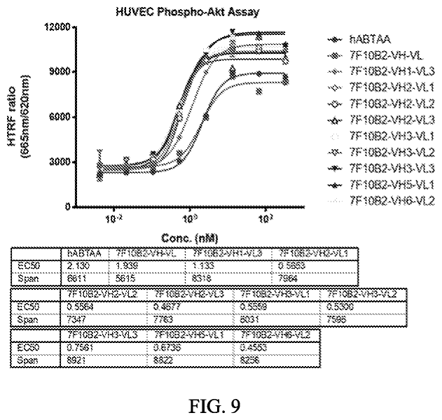

We also utilized the humanized ABTAA (hABTAA from U.S. Pat. No. 9,994,632B2) as reference antibody for comparison. As shown in FIGS. 8A-8C, for 5A7B9 humanization (FIG. 8A & 8B), all of humanized 5A7B9 leads showed lower EC₅₀ than hABTAA. In particular, humanized 5A7B9 leads of 5A7B9-VH3-VL1.1 and 5A7B9-VH3-VL2.1 showed 3-fold lower EC₅₀ than hABTAA. For 17F3E8 humanization (FIG. 8A), all of humanized 17F3E8 leads showed lower EC₅₀ than hABTAA. For 31E2D4 humanization (FIG. 8B), humanized 31E2D4 leads of 31E2D4-VH3-VL1, 31E2D4-VH4-VL1, 31E2D4-VH4-VL2 and 31E2D4-VH1-VL3 showed lower EC₅₀ than hABTAA. For 5F7D7 humanization (FIG. 8C), all of humanized 5F7D7 leads showed lower EC₅₀ than hABTAA. For 7F10B2 humanization (FIG. 9), all of humanized 7F10B2 leads showed lower EC₅₀ than hABTAA.

### Tie2-Ang2 Blockade Assay

The humanized antibodies were tested for the Tie2-Ang2 blockade bioassay as described in Example 2.

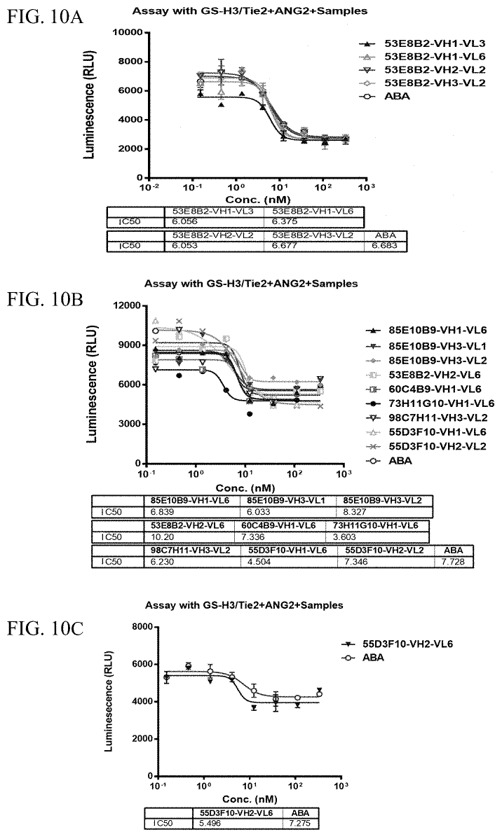

As shown in FIG. 10A, for 53E8B2 humanization, all of humanized 53E8B2 leads showed comparable blocking activity as compared to the reference antibody of ABA.

As shown in FIG. 10B, for 85E10B9 humanization, all of humanized 85E10B9 leads showed similar blocking activity as compared to ABA. For 53E8B2 humanization, the humanized lead of 53E8B2-VH2-VL6 showed similar blocking activity. For 60C4B9 humanization, the humanized lead of 60C4B9-VH1-VL6 showed similar blocking activity as compared to ABA. For 73H11G10 humanization, the humanized lead of 73H11G10-VH1-VL6 showed better blocking activity than the reference antibody of ABA that was revealed by lower EC₅₀ value. For 98C7H11 humanization, the humanized lead of 98C7H11-VH3-VL2 showed similar blocking activity as compared to ABA. For 55D3F10 humanization, one humanized lead of 55D3F10-VH1-VL6 showed better blocking activity than ABA, and the other one of 55D3F10-VH2-VL2 showed comparable blocking activity.

As shown in FIG. 10C, for 55D3F10 humanization, one humanized lead of 5D3F10-VH2-VL6 showed better blocking activity than ABA.

### Binding Affinity Analysis by Indirect ELISA

The humanized antibodies were tested for their binding activities to human Ang2 in an indirect ELISA. Briefly, 100 μl of 0.5 μg/ml human Ang2 proteins (R&D Systems; 623-AN-01M/CF) were pre-coated onto ELISA plates overnight at 4° C. On the next day, the plates were incubated with 100 μl 3× serially diluted primary antibodies, with initial concentration at 1 μg/ml. The plates were added with HRP-conjugated goat anti-human IgG antibody (Jackson, cat #109-605-098) and then TMB for chromogenic reaction. The antibody-antigen binding curves were generated with optical density readings at 450 nm. Antibody ABA and IgG1 were used as a positive and negative control respectively. Raw data was plotted using GraphPad Prism v6.02 software with four parameters, and best-fit values program was used to analyze the EC₅₀.

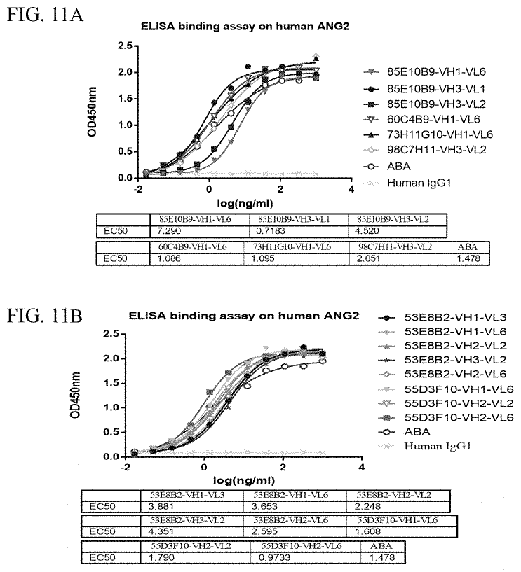

As shown in FIG. 11A and FIG. 11B, the humanized antibodies of 85E10B9-VH3-VL1, 60C4B9-VH1-VL6, 73H11G10-VH1-VL6 and 55D3F10-VH2-VL6 showed higher binding affinity than ABA, the humanized antibodies of 98C7H11-VH3-VL2, 53E8B2-VH1-VL3, 53E8B2-VH1-VL6, 53E8B2-VH2-VL2, 53E8B2-VH2-VL6, 55D3F10-VH1-VL6 and 55D3F10-VH2-VL2 showed comparable binding affinity when compared with ABA.

### SEQUENCES

The sequences of the disclosure were summarized in Table 1 and below.

1 > 43F1C10-2
VH-CDR1:
(SEQ ID NO: 1)
NYYMH
VH-CDR2:
(SEQ ID NO: 23)
YIHPNNGDTSYNQKFKG
VH-CDR3:
(SEQ ID NO: 60)
VSYSHYVAGAMDY
VL-CDR1:
(SEQ ID NO: 92)
KASQSVSNDVA
VL-CDR2:
(SEQ ID NO: 114)
FASNRYT
VL-CDR3:
(SEQ ID NO: 133)
QQDYSSPYT
VH:
(SEQ ID NO: 160)
EVQLHQSGPELVKPGASVKMSCKASGYTFTNYYMHWVKQSHGKSIEWIGYIHPNNGDT
SYNQKFKGKATLTVDKSSSTAYMELRSLTSEDSAVYYCAVVSYSHYVAGAMDYWGQG
TSVTVSS
VL:
(SEQ ID NO: 210)
SIVMTQTPKFLLVSAGDRVTITCKASQSVSNDVAWYQQKPGQSPKLLIYFASNRYTGVP
DRFTGSGYGTDFTFTISTVQAEDLAVYFCQQDYSSPYTFGGGTKLEIK
2 > 49E9G10
VH-CDR1:
(SEQ ID NO: 2)
DYGMV
VH-CDR2:
(SEQ ID NO: 24)
YIGSGRNTIYYVDTVKG
VH-CDR3:
(SEQ ID NO: 61)
EDNGYSYAMDY
VL-CDR1:
(SEQ ID NO: 93)
LASQTIGTWLA
VL-CDR2:
(SEQ ID NO: 115)
AATSLAD
VL-CDR3:
(SEQ ID NO: 134)
QQLYSIPLT
VH:
(SEQ ID NO: 161)
EVKLVESGGGLVQPGGSRKLSCAASGFTFSDYGMVWVRQAPGKGLEWVAYIGSGRNTI
YYVDTVKGRFTISRDNPKNTLFLQMSSLRSEDTAMYYCAREDNGYSYAMDYWGQGTS
VTVSS
VL:
(SEQ ID NO: 211)
DIQMTQSPASQSASLGESVTITCLASQTIGTWLAWYQQKPGKSPQLLIYAATSLADGVPS
RFSGSGSGTKFSFKISSLQAEDFGSYYCQQLYSIPLTFGVGTKLELK
3 > 50B9C10
VH-CDR1:
(SEQ ID NO: 3)
NYYIH
VH-CDR2:
(SEQ ID NO: 25)
YIYPNNGDTSYNQKFKG
VH-CDR3:
(SEQ ID NO: 62)
VSYSNYVAGAMDY
VL-CDR1:
(SEQ ID NO: 92)
KASQSVSNDVA
VL-CDR2:
(SEQ ID NO: 114)
FASNRYT
VL-CDR3:
(SEQ ID NO: 133)
QQDYSSPYT
VH:
(SEQ ID NO: 162)
EVQLHQSGPELVKPGASVKMSCKASGYTFTNYYIHWVKQSHGKSIEWIGYIYPNNGDTS
YNQKFKGKATLTVDKSSSTAYMELRSLTSEDSAVYYCAVVSYSNYVAGAMDYWGQG
TSVTVSS
VL:
(SEQ ID NO: 212)
SIVMTQTPKFLLVSAGDRVTITCKASQSVSNDVAWYQQKPGQSPKLLMYFASNRYTGV
PDRFTGSGYGTDFTFTISTVQAEDLAVYFCQQDYSSPYTFGGGTKLEIK
4 > 46F3H4
VH-CDR1:
(SEQ ID NO: 3)
NYYIH
VH-CDR2:
(SEQ ID NO: 26)
YIYPNNGDTTYNQKFKG
VH-CDR3:
(SEQ ID NO: 62)
VSYSNYVAGAMDY
VL-CDR1:
(SEQ ID NO: 92)
KASQSVSNDVA
VL-CDR2:
(SEQ ID NO: 114)
FASNRYT
VL-CDR3:
(SEQ ID NO: 133)
QQDYSSPYT
VH:
(SEQ ID NO: 163)
EVQLHQSGPELVKPGASVKMSCKASGYTFTNYYIHWVKQSHGKSIEWIGYIYPNNGDTT
YNQKFKGKATLTVDKSSSIAYMELRSLTSEDSAVYYCAVVSYSNYVAGAMDYWGQGT
SVTVSS
VL:
(SEQ ID NO: 210)
SIVMTQTPKFLLVSAGDRVTITCKASQSVSNDVAWYQQKPGQSPKLLIYFASNRYTGVP
DRFTGSGYGTDFTFTISTVQAEDLAVYFCQQDYSSPYTFGGGTKLEIK
5 > 4D8F2
VH-CDR1:
(SEQ ID NO: 4)
SDYAWN
VH-CDR2:
(SEQ ID NO: 27)
YISYSGSTSYNPSLKS
VH-CDR3:
(SEQ ID NO: 63)
GVRGDMDY
VL-CDR1:
(SEQ ID NO: 92)
KASQSVSNDVA
VL-CDR2:
(SEQ ID NO: 116)
YASNRYT
VL-CDR3:
(SEQ ID NO: 135)
QQDYSSPFT
VH:
(SEQ ID NO: 164)
DVQLQESGPGLVKPSQSLSLTCTVTGYSITSDYAWNWIRQFPGNKLEWMGYISYSGSTS
YNPSLKSRISITRDTSKNQFFLQLNSVTTEDTATYYCARGVRGDMDYWGQGTSVTVSS
VL:
(SEQ ID NO: 213)
SIVMTQTPKFLLVSAGDRVTITCKASQSVSNDVAWYQQKPGQSPKLLIYYASNRYTGVP
DRFTGSGYGTDFTFTISTVQAEDLAVYFCQQDYSSPFTFGSGTKLEIK
6 > 50H8G3
VH-CDR1:
(SEQ ID NO: 5)
DFYME
VH-CDR2:
(SEQ ID NO: 28)
ASRNKAKDYTTEYSASVKG
VH-CDR3:
(SEQ ID NO: 64)
GSYSNYTWFAY
VL-CDR1:
(SEQ ID NO: 92)
KASQSVSNDVA
VL-CDR2:
(SEQ ID NO: 114)
FASNRYT
VL-CDR3:
(SEQ ID NO: 133)
QQDYSSPYT
VH:
(SEQ ID NO: 165)
EVNLVESGGGLVQPGGSLRLSCATSGFTFSDFYMEWVRQPPGKRLEWIAASRNKAKDY
TTEYSASVKGRFIVSRDTSQSILYLQMNALRAEDTAIYYCARGSYSNYTWFAYWGQGTL
VTVSA
VL:
(SEQ ID NO: 210)
SIVMTQTPKFLLVSAGDRVTITCKASQSVSNDVAWYQQKPGQSPKLLIYFASNRYTGVP
DRFTGSGYGTDFTFTISTVQAEDLAVYFCQQDYSSPYTFGGGTKLEIK
7 > 5A7B9
VH-CDR1:
(SEQ ID NO: 6)
NYGVN
VH-CDR2:
(SEQ ID NO: 29)
WINSYSGVPTYADDFKG
VH-CDR3:
(SEQ ID NO: 65)
GENNYYGGSYD
VL-CDR1:
(SEQ ID NO: 92)
KASQSVSNDVA
VL-CDR2:
(SEQ ID NO: 116)
YASNRYT
VL-CDR3:
(SEQ ID NO: 136)
QQDYSSPLT
VH:
(SEQ ID NO: 166)
QIQLVQSGPELKKPGETVKISCKASGYTFTNYGVNWVKQAPGKGLKWMGWINSYSGVP
TYADDFKGRFAFSLETSASTAYLQINNLKNEDTATYFCARGENNYYGGSYDWGQGTTL
TVSS
VL:
(SEQ ID NO: 214)
SIVMTQTPKFLLVSAGDRVTITCKASQSVSNDVAWYQQKPGQSPKLLIYYASNRYTGVP
DRFTGSGYGTDFTFTISTVQAEDLAVYFCQQDYSSPLTFGAGTKLELK
8 > 5F7D7
VH-CDR1:
(SEQ ID NO: 1)
NYYMH
VH-CDR2:
(SEQ ID NO: 25)
YIYPNNGDTSYNQKFKG
VH-CDR3:
(SEQ ID NO: 62)
VSYSNYVAGAMDY
VL-CDR1:
(SEQ ID NO: 92)
KASQSVSNDVA
VL-CDR2:
(SEQ ID NO: 114)
FASNRYT
VL-CDR3:
(SEQ ID NO: 133)
QQDYSSPYT
VH:
(SEQ ID NO: 167)
EVQLHQSGPELVKPGASVKMSCKASGYTFTNYYMHWVKQSHGKSIEWIGYIYPNNGDT
SYNQKFKGKATLTVDKSSSTAYMELRSLTSEDSAVYYCAVVSYSNYVAGAMDYWGQG
TSVTVSS
VL:
(SEQ ID NO: 215)
SIVMTQTPKFLLVSAGDRVTVTCKASQSVSNDVAWYQQKPGQSPKLLIYFASNRYTGVP
DRFTGSGYGTDFTFTISTVQAEDLAVYFCQQDYSSPYTFGGGTKLEIK
9 > 7F10B2
VH-CDR1:
(SEQ ID NO: 7)
DYNMD
VH-CDR2:
(SEQ ID NO: 30)
TINPKNGETSDNQKFKA
VH-CDR3:
(SEQ ID NO: 66)
NVDYSNYLFFPMDY
VL-CDR1:
(SEQ ID NO: 92)
KASQSVSNDVA
VL-CDR2:
(SEQ ID NO: 117)
YASNRFT
VL-CDR3:
(SEQ ID NO: 137)
QQDYSSRT
VH:
(SEQ ID NO: 168)
EVQLQQSGPELVKPGSSVKISCKTSGYTFTDYNMDWVKQSHGKSLEWIGTINPKNGETS
DNQKFKAKATLTVDKSSSTAYMELRSLTSEDSAVYYCAKNVDYSNYLFFPMDYWGQG
TSVTVSS
VL:
(SEQ ID NO: 216)
SIVMTQTPKFLLVSAGDRVTITCKASQSVSNDVAWYQQKPGQSPKLLIYYASNRFTGVP
DRFTGSGYGTDFTFTITTVQAEDLAVYFCQQDYSSRTFGGGTKLEIK
10 > 31E2D4
VH-CDR1:
(SEQ ID NO: 8)
NYGMN
VH-CDR2:
(SEQ ID NO: 29)
WINSYSGVPTYADDFKG
VH-CDR3:
(SEQ ID NO: 65)
GENNYYGGSYD
VL-CDR1:
(SEQ ID NO: 92)
KASQSVSNDVA
VL-CDR2:
(SEQ ID NO: 116)
YASNRYT
VL-CDR3:
(SEQ ID NO: 136)
QQDYSSPLT
VH:
(SEQ ID NO: 169)
QIQLVQSGPELKKPGETVKISCKASGYTLTNYGMNWVKQAPGKGLKWMGWINSYSGV
PTYADDFKGRFAFSLETSASTAYLQINNLKNEDTATYFCARGENNYYGGSYDWGQGTT
LTVSS
VL:
(SEQ ID NO: 217)
SIVMTQTPKFLLVSAGDRVTITCKASQSVSNDVAWYQQKPGQSPKLLIYYASNRYTGVP
DRFTGSGYGTDFTFTISTVQAEDLAVYFCQQDYSSPLTFGAGTKLELR
11 > 15H10D12
VH-CDR1:
(SEQ ID NO: 4)
SDYAWN
VH-CDR2:
(SEQ ID NO: 31)
YINYSGSTDYNPSLKS
VH-CDR3:
(SEQ ID NO: 63)
GVRGDMDY
VL-CDR1:
(SEQ ID NO: 92)
KASQSVSNDVA
VL-CDR2:
(SEQ ID NO: 116)
YASNRYT
VL-CDR3:
(SEQ ID NO: 135)
QQDYSSPFT
VH:
(SEQ ID NO: 170)
DVQLQESGPGLVKPSQSLSLTCTVTGYSITSDYAWNWIRQFPGNRLEWMGYINYSGSTD
YNPSLKSRISITRDTSKNQFFLQLNSVTTEDTATYYCARGVRGDMDYWGQGTSVTVSS
VL:
(SEQ ID NO: 218)
SIVMTQTPKFLLVSAGDRVTITCKASQSVSNDVAWYQQKPGQSPKLLIYYASNRYTGVP
DRFTGSGYGTDFTFTISTVQADDLAVYFCQQDYSSPFTFGSGTKLEIK
12 > 25C5D6F3
VH-CDR1:
(SEQ ID NO: 5)
DFYME
VH-CDR2:
(SEQ ID NO: 32)
ASRNKANDYTTEYSASVKG
VH-CDR3:
(SEQ ID NO: 67)
GSYTNYTWFAY
VL-CDR1:
(SEQ ID NO: 92)
KASQSVSNDVA
VL-CDR2:
(SEQ ID NO: 114)
FASNRYT
VL-CDR3:
(SEQ ID NO: 133)
QQDYSSPYT
VH:
(SEQ ID NO: 171)
EVNLVESGGGLVQPGGSLRLSCATSGFTFSDFYMEWVRQPPGKRLEWIAASRNKANDY
TTEYSASVKGRFIVSRDTSQSILYLQMNALRAEDTAIYYCTRGSYTNYTWFAYWGQGTL
VTVSA
VL:
(SEQ ID NO: 210)
SIVMTQTPKFLLVSAGDRVTITCKASQSVSNDVAWYQQKPGQSPKLLIYFASNRYTGVP
DRFTGSGYGTDFTFTISTVQAEDLAVYFCQQDYSSPYTFGGGTKLEIK
13 > 17F3E8
VH-CDR1:
(SEQ ID NO: 3)
NYYIH
VH-CDR2:
(SEQ ID NO: 33)
YIHPNNGDTSYNQKFKD
VH-CDR3:
(SEQ ID NO: 68)
VSYSHYVAGALDN
VL-CDR1:
(SEQ ID NO: 92)
KASQSVSNDVA
VL-CDR2:
(SEQ ID NO: 114)
FASNRYT
VL-CDR3:
(SEQ ID NO: 133)
QQDYSSPYT
VH:
(SEQ ID NO: 172)
EVQLHQSGPELVKPGASVKMSCKASGYTFTNYYIHWVKQSHGKTIEWIGYIHPNNGDTS
YNQKFKDKATLTVDKSSSTAYMDLRSLTSEDSAVYYCSVVSYSHYVAGALDNWGQGT
SVTVSS
VL:
(SEQ ID NO: 210)
SIVMTQTPKFLLVSAGDRVTITCKASQSVSNDVAWYQQKPGQSPKLLIYFASNRYTGVP
DRFTGSGYGTDFTFTISTVQAEDLAVYFCQQDYSSPYTFGGGTKLEIK
14 > 82B10C1
VH-CDR1:
(SEQ ID NO: 9)
DYIIH
VH-CDR2:
(SEQ ID NO: 34)
YINPFSGGTNYNEKFKS
VH-CDR3:
(SEQ ID NO: 69)
WGPYYYSTYMRAYLMDA
VL-CDR1:
(SEQ ID NO: 94)
RTSQNVDNYGITYMY
VL-CDR2:
(SEQ ID NO: 118)
EGSNLAS
VL-CDR3:
(SEQ ID NO: 138)
QQSKDYPWT
VH:
(SEQ ID NO: 173)
QVKLLQSGAALVKPGDSVKMSCKASGYTFTDYIIHWVKQSHGKSLEWIGYINPFSGGTN
YNEKFKSKATLTVDKSSSTAYMEFSRLTSDDSAIYYCARWGPYYYSTYMRAYLMDAW
GQGASVTVSS
VL:
(SEQ ID NO: 219)
DIVLTQSPALAVSLEQRVTISCRTSQNVDNYGITYMYWYQQKSRQQPKLLIYEGSNLAS
GIPARFSGSGSGTDFTLTIDPVEADDIATYYCQQSKDYPWTFGGGTKLELK
15 > 50H8E5C5
VH-CDR1:
(SEQ ID NO: 1)
NYYMH
VH-CDR2:
(SEQ ID NO: 35)
YIYPNNGDTNYNQKFKG
VH-CDR3:
(SEQ ID NO: 62)
VSYSNYVAGAMDY
VL-CDR1:
(SEQ ID NO: 92)
KASQSVSNDVA
VL-CDR2:
(SEQ ID NO: 114)
FASNRYT
VL-CDR3:
(SEQ ID NO: 133)
QQDYSSPYT
VH:
(SEQ ID NO: 174)
EVQLHQSGPELVKPGASVKMSCKASGYTFTNYYMHWVKQSHGKSIEWIGYIYPNNGDT
NYNQKFKGKATLTVDKSSSTAYMELRSLTSEDSAVYSCAVVSYSNYVAGAMDYWGQG
TSVTVSS
VL:
(SEQ ID NO: 210)
SIVMTQTPKFLLVSAGDRVTITCKASQSVSNDVAWYQQKPGQSPKLLIYFASNRYTGVP
DRFTGSGYGTDFTFTISTVQAEDLAVYFCQQDYSSPYTFGGGTKLEIK
16 > 41E4F2
VH-CDR1:
(SEQ ID NO: 4)
SDYAWN
VH-CDR2:
(SEQ ID NO: 27)
YISYSGSTSYNPSLKS
VH-CDR3:
(SEQ ID NO: 70)
VIYYSDYGAMDY
VL-CDR1:
(SEQ ID NO: 95)
RASENIYSYLA
VL-CDR2:
(SEQ ID NO: 119)
NAKTLAE
VL-CDR3:
(SEQ ID NO: 139)
QHHYGTPWT
VH:
(SEQ ID NO: 175)
DVQLQESGPGLVKPSQSLSLTCTVTGYSITSDYAWNWIRQFPGNKLEWMGYISYSGSTS
YNPSLKSRISITRDTSKNQFFLQLNSVTTEDTATYYCASVIYYSDYGAMDYWGQGTSVT
VSS
VL:
(SEQ ID NO: 220)
DIQMTQSPASLSASVGETVTITCRASENIYSYLAWYQQKQGKSPQLLVYNAKTLAEGVP
SRFSGSGSGTQFSLKINSLQPEDFGSYYCQHHYGTPWTFGGGTKLEIK
17 > 41F7H8
VH-CDR1:
(SEQ ID NO: 4)
SDYAWN
VH-CDR2:
(SEQ ID NO: 27)
YISYSGSTSYNPSLKS
VH-CDR3:
(SEQ ID NO: 71)
CVYGNYVGAMDY
VL-CDR1:
(SEQ ID NO: 96)
KAGQSVSNDVA
VL-CDR2:
(SEQ ID NO: 116)
YASNRYT
VL-CDR3:
(SEQ ID NO: 136)
QQDYSSPLT
VH:
(SEQ ID NO: 176)
DVQFQESGPGLVKPSQSLSLTCTVTGYSITSDYAWNWIRQFPGNKLEWMGYISYSGSTS
YNPSLKSRISITRDTSKNQFFLQLNSVTTEDTATYYCARCVYGNYVGAMDYWGQGTSV
TVSS
VL:
(SEQ ID NO: 221)
SIVMTQTPKFLLVSAGDRVTITCKAGQSVSNDVAWYQQKPGQSPKLLIYYASNRYTGVP
DRFTGSGYGTDFTFTISTVQAEDLAVYFCQQDYSSPLTFGAGTKLELK
18 > 41H8A8
VH-CDR1:
(SEQ ID NO: 10)
SNWMH
VH-CDR2:
(SEQ ID NO: 36)
AIYPGNSDTSYNQKFKD
VH-CDR3:
(SEQ ID NO: 72)
EGITLVVATYWCFDV
VL-CDR1:
(SEQ ID NO: 97)
KASQNVGTNVA
VL-CDR2:
(SEQ ID NO: 120)
SASYRYS
VL-CDR3:
(SEQ ID NO: 140)
QQYNNYPYT
VH:
(SEQ ID NO: 177)
EVQLQQSGTELARPGASVKMSCKASGYTFTSNWMHWVKQRPGQGLEWIGAIYPGNSD
TSYNQKFKDKAKLTAVTSASTAYMELSSLTNEDSAVYYCTREGITLVVATYWCFDVWG
TGTTVTVSS
VL:
(SEQ ID NO: 222)
DIVTTQSQKFMSTSVGDRVSVTCKASQNVGTNVAWYQQKPGQSPKALIYSASYRYSGV
PDRFTGSGSGTDFTLTISNVQSEDLAEYFCQQYNNYPYTFGGGTKLEIK
19 > 42A2A6
VH-CDR1:
(SEQ ID NO: 4)
SDYAWN
VH-CDR2:
(SEQ ID NO: 37)
FINYSGTTSYNPSLKS
VH-CDR3:
(SEQ ID NO: 73)
WGYSDFLYYFDY
VL-CDR1:
(SEQ ID NO: 98)
RASSSVSYIH
VL-CDR2:
(SEQ ID NO: 121)
ATSNLAS
VL-CDR3:
(SEQ ID NO: 141)
QQWSNNPWT
VH:
(SEQ ID NO: 178)
DVQLQESGPGLVKPSQSLSLTCTVTGYSITSDYAWNWIRQFPGNKLEWMGFINYSGTTS
YNPSLKSRISLTRDTSKNQFFLQLNSVTTEDTATYYCARWGYSDFLYYFDYWGQGTTLT
VSS
VL:
(SEQ ID NO: 223)
QIVLSQSPAILSASPGEKVTMTCRASSSVSYIHWYQQKPGYSPKPWIYATSNLASGVPAR
FSGGGSGTSYSLTISRVEAEDAATYYCQQWSNNPWTFGGGTKLEIK
20 > 42C3A4
VH-CDR1:
(SEQ ID NO: 11)
NYWMD
VH-CDR2:
(SEQ ID NO: 38)
EIRLKSNNYATHYAESVKG
VH-CDR3:
(SEQ ID NO: 74)
GAPLFGGYYKGVYFDY
VL-CDR1:
(SEQ ID NO: 99)
QASQSVSNEVA
VL-CDR2:
(SEQ ID NO: 122)
YASSRYT
VL-CDR3:
(SEQ ID NO: 142)
QQDYNSPYT
VH:
(SEQ ID NO: 179)
EVKLEESGGGLVQPGGSMKLSCVASGITFSNYWMDWVRQSPEKGLEWVAEIRLKSNNY
ATHYAESVKGRFTISRDDSKNSVYLQMNNLRAEDTGIYYCTRGAPLFGGYYKGVYFDY
WGQGTTLTVSS
VL:
(SEQ ID NO: 224)
SFVMTQTPKSLLASAGDRVTITCQASQSVSNEVAWYQQKPGQSPKLLIYYASSRYTGVP
DRFAGSGYGTDFTFTISTVQAEDLAVYFCQQDYNSPYTFGGGTKLEIK
21 > 43B2A12
VH-CDR1:
(SEQ ID NO: 12)
GYWMH
VH-CDR2:
(SEQ ID NO: 39)
EIDPSDSFTYNNQKFKG
VH-CDR3:
(SEQ ID NO: 75)
SGLLRWFAYW
VL-CDR1:
(SEQ ID NO: 100)
RASSSVNYMY
VL-CDR2:
(SEQ ID NO: 123)
YTSHLAP
VL-CDR3:
(SEQ ID NO: 143)
QQFTSSPYT
VH:
(SEQ ID NO: 180)
QVQLQQPGAELVKPGASVKLSCKASGYNFTGYWMHWVKQRPGQGLEWIGEIDPSDSF
TYNNQKFKGKATLTVDKSSNTAYMQLSSLTSEDSAVYYCARSGLLRWFAYWGQGTLV
TVSA
VL:
(SEQ ID NO: 225)
ENVLTQSPAIMSASLGEKVTMSCRASSSVNYMYWYQQKSYASPKLWIYYTSHLAPGVP
GRFSGSGSGNSYFLTISSMEGEDAATYYCQQFTSSPYTFGGGTKLEIK
22 > 43C4A11
VH-CDR1:
(SEQ ID NO: 13)
SYWMH
VH-CDR2:
(SEQ ID NO: 40)
EIDPSDSYSYYNQKFKG
VH-CDR3:
(SEQ ID NO: 76)
RWGYRYYAMDY
VL-CDR1:
(SEQ ID NO: 101)
KASQNVGTAVA
VL-CDR2:
(SEQ ID NO: 124)
SASNRYT
VL-CDR3:
(SEQ ID NO: 144)
QQYSSYPYT
VH:
(SEQ ID NO: 181)
QVQLQQPGAELVKPGASVKLSCKASGYTFTSYWMHWVKQRPGQGLEWIGEIDPSDSYS
YYNQKFKGKATLTVDKSSSTAYMQLSSLTSEDSAVYYCARRWGYRYYAMDYWGQGT
SVTVSS
VL:
(SEQ ID NO: 226)
DIVMTQSQKFMSTSVGDRVSITCKASQNVGTAVAWYQQKPGQSPKSLIYSASNRYTGV
PDRLTGSGSGTDFTLTISNMQSEDLADYFCQQYSSYPYTFGGGTKLEIK
23 > 43D3A9
VH-CDR1:
(SEQ ID NO: 14)
SNWIH
VH-CDR2:
(SEQ ID NO: 41)
AIYPGNNDTSYNQKFKD
VH-CDR3:
(SEQ ID NO: 77)
EGVTMVVATYWCFDV
VL-CDR1:
(SEQ ID NO: 97)
KASQNVGTNVA
VL-CDR2:
(SEQ ID NO: 120)
SASYRYS
VL-CDR3:
(SEQ ID NO: 145)
QQYNNFPYT
VH:
(SEQ ID NO: 182)
EVQLQQSGAELARPGASVKMSCKASGYTFTSNWIHWVKQRPGQGLEWIGAIYPGNNDT
SYNQKFKDKAKLTAVTSANTAYMELSSLTNEDSAVYYCTREGVTMVVATYWCFDVW
GTGTTVTVSS
VL:
(SEQ ID NO: 227)
DIVMTQSQKFMSTSVGDRVSVTCKASQNVGTNVAWYQQKPGQSPKALIYSASYRYSGV
PDRFTGSGSGTDFTLTISNVQSEDLAEYFCQQYNNFPYTFGGGTKLEIK
24 > 43F1C10
VH-CDR1:
(SEQ ID NO: 5)
DFYME
VH-CDR2:
(SEQ ID NO: 42)
ASRDKANDYTTEYSASVKG
VH-CDR3:
(SEQ ID NO: 64)
GSYSNYTWFAY
VL-CDR1:
(SEQ ID NO: 92)
KASQSVSNDVA
VL-CDR2:
(SEQ ID NO: 114)
FASNRYT
VL-CDR3:
(SEQ ID NO: 133)
QQDYSSPYT
VH:
(SEQ ID NO: 183)
EVNLVESGGGLVQPGGSLRLSCATSGFTFSDFYMEWVRQPPGKRLEWIAASRDKANDY
TTEYSASVKGRFIVSRDTSQSILYLQMNALRAEDTAIYYCARGSYSNYTWFAYWGQGTL
VTVSA
VL:
(SEQ ID NO: 210)
SIVMTQTPKFLLVSAGDRVTITCKASQSVSNDVAWYQQKPGQSPKLLIYFASNRYTGVP
DRFTGSGYGTDFTFTISTVQAEDLAVYFCQQDYSSPYTFGGGTKLEIK
25 > 43G5G3
VH-CDR1:
(SEQ ID NO: 5)
DFYME
VH-CDR2:
(SEQ ID NO: 43)
SSRNKANDYTTEYNASVKG
VH-CDR3:
(SEQ ID NO: 78)
ANDGYWFAY
VL-CDR1:
(SEQ ID NO: 102)
LASQTVGTWLA
VL-CDR2:
(SEQ ID NO: 115)
AATSLAD
VL-CDR3:
(SEQ ID NO: 146)
QQIFSVPYT
VH:
(SEQ ID NO: 184)
EVNLVESGGGLVQPGGSLRLSCATSGFTFSDFYMEWVRQPPGKRLEWIASSRNKANDY
TTEYNASVKGRFIVSRDTSQSILYLQMNALRAEDTAIYYCARANDGYWFAYWGQGTLV
TVSA
VL:
(SEQ ID NO: 228)
DIQMTQSPASQSASLGESVTITCLASQTVGTWLAWYQQKPGKSPQLLIFAATSLADGVPS
RFSGSGSGTKFSFKISSLQAEDFVSYYCQQIFSVPYTFGGGTKLEIK
26 > 45C5A1
VH-CDR1:
(SEQ ID NO: 15)
NYTMS
VH-CDR2:
(SEQ ID NO: 44)
EISNSDGSAYYPDTVKG
VH-CDR3:
(SEQ ID NO: 79)
HGGLRVYYAIDY
VL-CDR1:
(SEQ ID NO: 103)
LASQTIGPWLA
VL-CDR2:
(SEQ ID NO: 115)
AATSLAD
VL-CDR3:
(SEQ ID NO: 147)
QQLYSDPLYT
VH:
(SEQ ID NO: 185)
EVKLVESGGGLVQPGGSLKLSCAASGFTFSNYTMSWVRQTPEKRLELVAEISNSDGSAY
YPDTVKGRFTISRDNAKKTLYLQMSSLKSEDTAMYYCARHGGLRVYYAIDYWGQGTS
VTVSS
VL:
(SEQ ID NO: 229)
DIQMTQSPASQSASLGESVTITCLASQTIGPWLAWYQQKPGKSPQLLIYAATSLADGVPS
RFSGSGSGTKFSFKISSLQAEDFVSYYCQQLYSDPLYTFGGGTKLEMK
27 > 45C9F9
VH-CDR1:
(SEQ ID NO: 8)
NYGMN
VH-CDR2:
(SEQ ID NO: 45)
WISTYSGVPTYADDFKG
VH-CDR3:
(SEQ ID NO: 80)
FGALMGYYVGFAY
VL-CDR1:
(SEQ ID NO: 104)
KASEDIYNRLA
VL-CDR2:
(SEQ ID NO: 125)
GATSLET
VL-CDR3:
(SEQ ID NO: 148)
QQYWSTPPYT
VH:
(SEQ ID NO: 186)
QIQLVQSGPELKKPGETVKISCKASGYTFTNYGMNWVKQAPGKGLKWMGWISTYSGVP
TYADDFKGRFAFSLETSASTAYLQINNLKNEDSATYFCSRFGALMGYYVGFAYWGQGT
LVTVSA
VL:
(SEQ ID NO: 230)
DIQMTQSSSSFSVSLGDRVTITCKASEDIYNRLAWYQQKPGNAPRLLISGATSLETGVPSR
FSGSGSGKDYTLSITSLQTEDVATYYCQQYWSTPPYTFGGGTKLEIK
28 > 45C9F9-2
VH-CDR1:
(SEQ ID NO: 16)
SYGVH
VH-CDR2:
(SEQ ID NO: 46)
VIWAGGSTNYNSALMS
VH-CDR3:
(SEQ ID NO: 81)
DQLDGFDY
VL-CDR1:
(SEQ ID NO: 104)
KASEDIYNRLA
VL-CDR2:
(SEQ ID NO: 125)
GATSLET
VL-CDR3:
(SEQ ID NO: 148)
QQYWSTPPYT
VH:
(SEQ ID NO: 187)
QVQLKESGPGLVAPSQSLSITCTVSGFSLTSYGVHWVRQPPGKGLEWLGVIWAGGSTNY
NSALMSRLSISKDNSKSQVFLKMNSLQTDDTAMYYCARDQLDGFDYWGQGTTLTVSS
VL:
(SEQ ID NO: 230)
DIQMTQSSSSFSVSLGDRVTITCKASEDIYNRLAWYQQKPGNAPRLLISGATSLETGVPSR
FSGSGSGKDYTLSITSLQTEDVATYYCQQYWSTPPYTFGGGTKLEIK
29 > 46H3H3
VH-CDR1:
(SEQ ID NO: 2)
DYGMV
VH-CDR2:
(SEQ ID NO: 47)
YIGSGRSTIYYADTVKG
VH-CDR3:
(SEQ ID NO: 61)
EDNGYSYAMDY
VL-CDR1:
(SEQ ID NO: 93)
LASQTIGTWLA
VL-CDR2:
(SEQ ID NO: 115)
AATSLAD
VL-CDR3:
(SEQ ID NO: 149)
QQLYSTPLT
VH:
(SEQ ID NO: 188)
EVKLVESGGGLVQPGGSRKLSCAASGFTFSDYGMVWVRQAPGKGLEWVAYIGSGRSTI
YYADTVKGRFTISRDNPKNTLFLQMSSLRSEDTAMYYCAREDNGYSYAMDYWGQGTS
VTVSS
VL:
(SEQ ID NO: 231)
DIQMTQSPASQSASLGESVTITCLASQTIGTWLAWYQQKPGKSPQLLIYAATSLADGVPS
RFSGSGSGTKFSFKISSLQAEDFVSYYCQQLYSTPLTFGAGTKLELK
30 > 49C3B8-VH
VH-CDR1:
(SEQ ID NO: 17)
SDNWN
VH-CDR2:
(SEQ ID NO: 48)
YISYSGSSYYNPSLKS
VH-CDR3:
(SEQ ID NO: 82)
GLSHAMDY
VL-CDR1:
(SEQ ID NO: 105)
RSSQSIVHTNGNTYLE
VL-CDR2:
(SEQ ID NO: 126)
KVSNRFS
VL-CDR3:
(SEQ ID NO: 150)
FQGSHVPPT
VH:
(SEQ ID NO: 189)
EVQLQESGPSLVKPSQTLSLTCSVTGDSITSDNWNWIRKFPGNKLEYMGYISYSGSSYYN
PSLKSRISITRDTSKNQYYLQLNSVTSEDTATYYCARGLSHAMDYWGQGTSVTVSS
VL:
(SEQ ID NO: 232)
DVLMTQTPLSLPVSLGDQASISCRSSQSIVHTNGNTYLEWYLQKPGQSPKLLIYKVSNRF
SGVPDRFSGSGSGTDFTLKISRVEAEDLGVYYCFQGSHVPPTFGGGTKLEIK
31 > 49E4C2
VH-CDR1:
(SEQ ID NO: 11)
NYWMD
VH-CDR2:
(SEQ ID NO: 38)
EIRLKSNNYATHYAESVKG
VH-CDR3:
(SEQ ID NO: 83)
GAPLFDGYYKGVYFDY
VL-CDR1:
(SEQ ID NO: 106)
KASQSVSNEVA
VL-CDR2:
(SEQ ID NO: 116)
YASNRYT
VL-CDR3:
(SEQ ID NO: 142)
QQDYNSPYT
VH:
(SEQ ID NO: 190)
EVKLEESGGGLVQPGGSMKLSCVASGITFSNYWMDWVRQSPEKGLEWVAEIRLKSNNY
ATHYAESVKGRFTISRDDSKSSVYLQMNNLRAEDTGIYYCTRGAPLFDGYYKGVYFDY
WGQGTTLTVSS
VL:
(SEQ ID NO: 233)
STVMTQTPKSLLASAGDRVAITCKASQSVSNEVAWYQQKPGLSPKLLIYYASNRYTGVP
DRFTGSGYGTDFTFTISTVQAEDLAVYFCQQDYNSPYTFGGGTKLEIK
32 > 49E4F1
VH-CDR1:
(SEQ ID NO: 10)
SNWMH
VH-CDR2:
(SEQ ID NO: 49)
AIYPGNSDTDYNQKFKD
VH-CDR3:
(SEQ ID NO: 84)
EGITLVVTSYWCFDV
VL-CDR1:
(SEQ ID NO: 97)
KASQNVGTNVA
VL-CDR2:
(SEQ ID NO: 120)
SASYRYS
VL-CDR3:
(SEQ ID NO: 151)
QQYNSYPYT
VH:
(SEQ ID NO: 191)
EVQLQQSGTELARPGASVKMSCKASGYTFTSNWMHWVKQRPGQGLEWIGAIYPGNSD
TDYNQKFKDKAKLTAVTSASTAYMELSSLTDEDSAVYYCTREGITLVVTSYWCFDVWG
TGTTVTVSS
VL:
(SEQ ID NO: 234)
DIVMTQSQKFMSTSVGDRVSVTCKASQNVGTNVAWYQQKPGQSPKALIYSASYRYSGV
PDRFTGSGSGTDFTLTISNVQSEDLAEYFCQQYNSYPYTFGGGTKLEIK
33 > 49G4D6
VH-CDR1:
(SEQ ID NO: 2)
DYGMV
VH-CDR2:
(SEQ ID NO: 50)
YIGSGRNTIYYADTVKG
VH-CDR3:
(SEQ ID NO: 61)
EDNGYSYAMDY
VL-CDR1:
(SEQ ID NO: 93)
LASQTIGTWLA
VL-CDR2:
(SEQ ID NO: 115)
AATSLAD
VL-CDR3:
(SEQ ID NO: 134)
QQLYSIPLT
VH:
(SEQ ID NO: 192)
EVKLVESGGGLVQPGGSRKLSCAASGFTFSDYGMVWVRQAPGKGLEWVAYIGSGRNTI
YYADTVKGRFTISRDNPKNTLFLQMSSLRSEDTAMYYCAREDNGYSYAMDYWGQGTS
VTVSS
VL:
(SEQ ID NO: 235)
DIQMTQSPASQSASLGESVTITCLASQTIGTWLAWYQQKPGKSPQLLIYAATSLADGVPS
RFSSSGSGTKFSFKISSLQAEDFVSYYCQQLYSIPLTFGAGTKLELK
34 > 4D11H2
VH-CDR1:
(SEQ ID NO: 10)
SNWMH
VH-CDR2:
(SEQ ID NO: 36)
AIYPGNSDTSYNQKFKD
VH-CDR3:
(SEQ ID NO: 85)
EGITMVVATYWCFDV
VL-CDR1:
(SEQ ID NO: 97)
KASQNVGTNVA
VL-CDR2:
(SEQ ID NO: 120)
SASYRYS
VL-CDR3:
(SEQ ID NO: 152)
QQYNSFPYT
VH:
(SEQ ID NO: 193)
EVQLQQSGTELARPGASVKMSCKASGYTFTSNWMHWVKQRPGQGLEWIGAIYPGNSD
TSYNQKFKDKAKLTAVTSASTAYMELSSLTNEDSAVYYCTREGITMVVATYWCFDVW
GTGTTVTVSS
VL:
(SEQ ID NO: 236)
DIVMTQSQKFMSTSVGDRVSVTCKASQNVGTNVAWYQQKPGQSPKALIYSASYRYSGV
PDRFTGSGSGTDFTLTISNVQSEDLAEYFCQQYNSFPYTFGGGTKLEIK
35 > 50B9C10-2
VH-CDR1:
(SEQ ID NO: 5)
DFYME
VH-CDR2:
(SEQ ID NO: 32)
ASRNKANDYTTEYSASVKG
VH-CDR3:
(SEQ ID NO: 86)
GSYRNYTWFAY
VL-CDR1:
(SEQ ID NO: 92)
KASQSVSNDVA
VL-CDR2:
(SEQ ID NO: 114)
FASNRYT
VL-CDR3:
(SEQ ID NO: 133)
QQDYSSPYT
VH:
(SEQ ID NO: 194)
EVNLVESGGGLVQPGGSLRLSCATSGFTFSDFYMEWVRQPPGKRLEWIAASRNKANDY
TTEYSASVKGRFIVSRDTSQSILYLQMNALRAEDTAIYYCARGSYSNYTWFAYWGQGT
LVTVSA
VL:
(SEQ ID NO: 212)
SIVMTQTPKFLLVSAGDRVTITCKASQSVSNDVAWYQQKPGQSPKLLMYFASNRYTGV
PDRFTGSGYGTDFTFTISTVQAEDLAVYFCQQDYSSPYTFGGGTKLEIK
36 > 50C9C2
VH-CDR1:
(SEQ ID NO: 18)
NYMMS
VH-CDR2:
(SEQ ID NO: 51)
EISNSDGSTYYPDTVKG
VH-CDR3:
(SEQ ID NO: 87)
HGGLRVYYAMDY
VL-CDR1:
(SEQ ID NO: 103)
LASQTIGPWLA
VL-CDR2:
(SEQ ID NO: 115)
AATSLAD
VL-CDR3:
(SEQ ID NO: 147)
QQLYSDPLYT
VH:
(SEQ ID NO: 195)
EVKLVESGGGLVQPGGSLKLSCAASGFTFSNYMMSWVRQTPEKRLELVAEISNSDGSTY
YPDTVKGRFTISRDNAKNTLYLQMSSLKSEDTAMYYCARHGGLRVYYAMDYWGQGTS
VTVSS
VL:
(SEQ ID NO: 229)
DIQMTQSPASQSASLGESVTITCLASQTIGPWLAWYQQKPGKSPQLLIYAATSLADGVPS
RFSGSGSGTKFSFKISSLQAEDFVSYYCQQLYSDPLYTFGGGTKLEMK
37 > 50F11D12
VH-CDR1:
(SEQ ID NO: 2)
DYGMV
VH-CDR2:
(SEQ ID NO: 52)
YISGGSKTVYYADTVKG
VH-CDR3:
(SEQ ID NO: 88)
EGLRTYYYAMDY
VL-CDR1:
(SEQ ID NO: 103)
LASQTIGPWLA
VL-CDR2:
(SEQ ID NO: 115)
AATSLAD
VL-CDR3:
(SEQ ID NO: 149)
QQLYSTPLT
VH:
(SEQ ID NO: 196)
EVKLVESGGGSVQPGGSRKLSCAASGFTFSDYGMVWVRQAPGKGLEWIGYISGGSKTV
YYADTVKGRFTMSRDNPQNTLFLQMSSLRSEDTAMYYCAREGLRTYYYAMDYWGQG
TSVTVSS
VL:
(SEQ ID NO: 237)
DIQMTQSPASQSASLGESVTITCLASQTIGPWLAWYQQKPGKSPQLLIYAATSLADGVPS
RFSGSGSGTKFSFKISSLQAEDFVSYYCQQLYSTPLTFGGGTKLEIK
38 > 54E4A12
VH-CDR1:
(SEQ ID NO: 19)
TYNVH
VH-CDR2:
(SEQ ID NO: 53)
VIWNTGGTRYNSTLKS
VH-CDR3:
(SEQ ID NO: 89)
GGGETGDY
VL-CDR1:
(SEQ ID NO: 107)
RASQNIYKYLN
VL-CDR2:
(SEQ ID NO: 127)
YTNSLQT
VL-CDR3:
(SEQ ID NO: 153)
FQYSSWYT
VH:
(SEQ ID NO: 197)
QVQLKESGPGLVQPSQTLSLTCTVAGFSLTTYNVHWVRQPPGKGLEWMGVIWNTGGTR
YNSTLKSRLSISKDTSKSQVFLKMNSLQTEDTATYYCARGGGETGDYWGQGVMVTVSS
VL:
(SEQ ID NO: 248)
DIQMTQSPSLLSASVGDRVTLSCRASQNIYKYLNWYQQKLGEAPKLLIHYTNSLQTGIPS
RFSGSGSGTDFTLTISSLQPEDVAIYFCFQYSSWYTFGAGTKLELK
39 > 5F7D7-2
VH-CDR1:
(SEQ ID NO: 5)
DFYME
VH-CDR2:
(SEQ ID NO: 32)
ASRNKANDYTTEYSASVKG
VH-CDR3:
(SEQ ID NO: 64)
GSYSNYTWFAY
VL-CDR1:
(SEQ ID NO: 92)
KASQSVSNDVA
VL-CDR2:
(SEQ ID NO: 114)
FASNRYT
VL-CDR3:
(SEQ ID NO: 133)
QQDYSSPYT
VH:
(SEQ ID NO: 198)
EVNLVESGGGLVQPGGSLRLSCATSGFTFSDFYMEWVRQPPGKRLEWIAASRNKANDY
TTEYSASVKGRFIVSRDTSQSILYLQMNALRAEDTAIYYCARGSYSNYTWFAYWGQGTL
VTVSA
VL:
(SEQ ID NO: 215)
SIVMTQTPKFLLVSAGDRVTVTCKASQSVSNDVAWYQQKPGQSPKLLIYFASNRYTGVP
DRFTGSGYGTDFTFTISTVQAEDLAVYFCQQDYSSPYTFGGGTKLEIK
40 > 76B2E6
VH-CDR1:
(SEQ ID NO: 20)
RFYIY
VH-CDR2:
(SEQ ID NO: 54)
YIYPGNGDTDYSEKFKG
VH-CDR3:
(SEQ ID NO: 90)
RTTAGIRFAY
VL-CDR1:
(SEQ ID NO: 108)
RSSQSFVHSDGNTYLN
VL-CDR2:
(SEQ ID NO: 128)
NISNRLS
VL-CDR3:
(SEQ ID NO: 154)
GQASKIPLT
VH:
(SEQ ID NO: 199)
QVHLQQSGAELATPGTSVKLSCKASGYSFTRFYIYWVKQRPGQGLEWIGYIYPGNGDTD
YSEKFKGKATLTTDTSSSTAYLLLGSLTPEDSAYYFCATRTTAGIRFAYWGQGTLVTVSS
VL:
(SEQ ID NO: 238)
DVVMTQTPVSLSVSLGGQVSISCRSSQSFVHSDGNTYLNWFLQKPGQSPQLLIYNISNRL
SGVPDRFSGSGSGTDFTLKISRVEHDDLGVFYCGQASKIPLTFGSGTKLEIK
41 > 41A6A6
VH-CDR1:
(SEQ ID NO: 2)
DYGMV
VH-CDR2:
(SEQ ID NO: 47)
YIGSGRSTIYYADTVKG
VH-CDR3:
(SEQ ID NO: 61)
EDNGYSYAMDY
VL-CDR1:
(SEQ ID NO: 93)
LASQTIGTWLA
VL-CDR2:
(SEQ ID NO: 115)
AATSLAD
VL-CDR3:
(SEQ ID NO: 134)
QQLYSIPLT
VH:
(SEQ ID NO: 200)
EVKLVESGGGLVQPGGSRKLSCAASGFTFSDYGMVWVRQAPGKGLEWVAYIGSGRSTI
YYADTVKGRFTISRDNPKNTLFLQMSSLGSEDTAMYYCAREDNGYSYAMDYWGQGTS
VTVSS
VL:
(SEQ ID NO: 239)
DIQMTQSPASQSASLGESVTITCLASQTIGTWLAWYQQKPGKSPQLLIYAATSLADGVPS
RFSGSGSGTKFSFKISSLQAEDFVSYYCQQLYSIPLTFGAGTKLELK
42 > 39C1C4
VH-CDR1:
(SEQ ID NO: 2)
DYGMV
VH-CDR2:
(SEQ ID NO: 50)
YIGSGRNTIYYADTVKG
VH-CDR3:
(SEQ ID NO: 61)
EDNGYSYAMDY
VL-CDR1:
(SEQ ID NO: 93)
LASQTIGTWLA
VL-CDR2:
(SEQ ID NO: 115)
AATSLAD
VL-CDR3:
(SEQ ID NO: 134)
QQLYSIPLT
VH:
(SEQ ID NO: 201)
EVKLVESGGGLVQPGGSRKLSCAASGFTFSDYGMVWVRQAPGKGLEWVAYIGSGRNTI
YYADTVKGRFTISRDNPKNTLFLQMSSLRSEDTAMYYCVREDNGYSYAMDYWGQGTS
VTVSS
VL:
(SEQ ID NO: 239)
DIQMTQSPASQSASLGESVTITCLASQTIGTWLAWYQQKPGKSPQLLIYAATSLADGVPS
RFSGSGSGTKFSFKISSLQAEDFVSYYCQQLYSIPLTFGAGTKLELK
43 > 49B8G4
VH-CDR1:
(SEQ ID NO: 2)
DYGMV
VH-CDR2:
(SEQ ID NO: 50)
YIGSGRNTIYYADTVKG
VH-CDR3:
(SEQ ID NO: 61)
EDNGYSYAMDY
VL-CDR1:
(SEQ ID NO: 93)
LASQTIGTWLA
VL-CDR2:
(SEQ ID NO: 115)
AATSLAD
VL-CDR3:
(SEQ ID NO: 134)
QQLYSIPLT
VH:
(SEQ ID NO: 202)
EVKLVESGGGLVQPGGSRKLSCAASGFTFSDYGMVWVRQAPGKGLEWIAYIGSGRNTI
YYADTVKGRFTISRDNPKNTLFLQMSSLRSEDTAMYYCAREDNGYSYAMDYWGQGTS
VTVSS
VL:
(SEQ ID NO: 240)
DIQMTQSPASQSASLGESVTVTCLASQTIGTWLAWYQQKPGKSPQLLIYAATSLADGVP
SRFSGSGSGTKFSFKISSLQAEDFGSYYCQQLYSIPLTFGVGTKLELK
44 > 27H11B5
VH-CDR1:
(SEQ ID NO: 4)
SDYAWN
VH-CDR2:
(SEQ ID NO: 27)
YISYSGSTSYNPSLKS
VH-CDR3:
(SEQ ID NO: 71)
CVYGNYVGAMDY
VL-CDR1:
(SEQ ID NO: 109)
KASQSVTNDVA
VL-CDR2:
(SEQ ID NO: 116)
YASNRYT
VL-CDR3:
(SEQ ID NO: 136)
QQDYSSPLT
VH-
(SEQ ID NO: 203)
DVQLQESGPGLVKPSQSLSLTCTVTGYSITSDYAWNWIRQFPGNKLEWMGYISYSGSTS
YNPSLKSRISITRDTSKNQFFLQLNSVTTEDTATYYCARCVYGNYVGAMDYWGQGTSV
TVSS
VL:
(SEQ ID NO: 241)
SIVMTQTPKFLLVSAGDRVTITCKASQSVTNDVAWYQQKPGQSPKLLIYYASNRYTGVP
DRFTGSGYGTDFTFTISTVQTEDLAVYFCQQDYSSPLTFGAGTKLELK
45 > 53E8B2
VH-CDR1:
(SEQ ID NO: 21)
DYAMA
VH-CDR2:
(SEQ ID NO: 55)
TIIYDGSSTYYRDSVKG
VH-CDR3:
(SEQ ID NO: 91)
HGRVFGFGYFDY
VL-CDR1:
(SEQ ID NO: 110)
QASQDIGDWVA
VL-CDR2:
(SEQ ID NO: 129)
GADRLAD
VL-CDR3:
(SEQ ID NO: 155)
LQDYSPPFT
VH:
(SEQ ID NO: 204)
EVQLVESGGGLVQPGRSLKFSCAASGFTFSDYAMAWVRQAPKKGLEWVATIIYDGSST
YYRDSVKGRFTISRDNAKSTLYLQMDSLRSEDTATYYCSRHGRVFGFGYFDYWGQGV
MVTVSS
VL:
(SEQ ID NO: 242)
DIQMTQSPASLSASLEEIVTITCQASQDIGDWVAWFQQRPGKSPQLLIFGADRLADGVPS
RFSGSRSGTQFLLKINRLQVEDPGIYYCLQDYSPPFTFGPGTQLELK
46 > 60C4B9
VH-CDR1:
(SEQ ID NO: 21)
DYAMA
VH-CDR2:
(SEQ ID NO: 56)
TIIHNGGTTFYRDSVKG
VH-CDR3:
(SEQ ID NO: 91)
HGRVFGFGYFDY
VL-CDR1:
(SEQ ID NO: 111)
QASQDIGDWLA
VL-CDR2:
(SEQ ID NO: 130)
GATSLAD
VL-CDR3:
(SEQ ID NO: 156)
LQDSDPPYT
VH:
(SEQ ID NO: 205)
EVQLVESGGGLVQPGRSLKFSCAASGFTFSDYAMAWVRQAPKKGLEWVATIIHNGGTT
FYRDSVKGRFTISRDNAKTTLYLQMDILRSEDTATYYCVRHGRVFGFGYFDYWGQGVM
VTVSS
VL:
(SEQ ID NO: 243)
DIQMTQSPASLSASLEEIVTITCQASQDIGDWLAWFHQKPGKSPHLLIYGATSLADGVPS
RFSGSGSGTQFSLRISGLQVEDPGIYYCLQDSDPPYTFGAGTKLELK
47 > 73H11G10
VH-CDR1:
(SEQ ID NO: 21)
DYAMA
VH-CDR2:
(SEQ ID NO: 55)
TIIYDGSSTYYRDSVKG
VH-CDR3:
(SEQ ID NO: 346)
HGRIFGFGYFDY
VL-CDR1:
(SEQ ID NO: 110)
QASQDIGDWVA
VL-CDR2:
(SEQ ID NO: 129)
GADRLAD
VL-CDR3:
(SEQ ID NO: 155)
LQDYSPPFT
VH:
(SEQ ID NO: 206)
EVQLVESGGGLVQPGRSLKFSCAASGFTFSDYAMAWVRQAPKKGLEWVATIIYDGSST
YYRDSVKGRFTISRDNAKSTLYLQMESLRPEDTATYHCSRHGRIFGFGYFDYWGQGVM
VTVSS
VL:
(SEQ ID NO: 242)
DIQMTQSPASLSASLEEIVTITCQASQDIGDWVAWFQQRPGKSPQLLIFGADRLADGVPS
RFSGSRSGTQFLLKINRLQVEDPGIYYCLQDYSPPFTFGPGTQLELK
48 > 15B4C6
VH-CDR1:
(SEQ ID NO: 22)
GYGVN
VH-CDR2:
(SEQ ID NO: 57)
LIWNNGSTDYNSALKS
VH-CDR3:
(SEQ ID NO: 347)
DPLYLLRGAMDY
VL-CDR1:
(SEQ ID NO: 112)
RASKSVSSSGYNFIY
VL-CDR2:
(SEQ ID NO: 131)
LASNLES
VL-CDR3:
(SEQ ID NO: 157)
QHSRDLPLT
VH:
(SEQ ID NO: 207)
QVQLKESGPGLVAPSQSLSITCTVSGFSLNGYGVNWVRQPPGKGLEWLGLIWNNGSTD
YNSALKSRLTISKDNSKSLVFLKMNSLQTDDTARYYCARDPLYLLRGAMDYWGQGTSV
TVSS
VL:
(SEQ ID NO: 244)
DIVLTQSPASLTVSLGQRATISCRASKSVSSSGYNFIYWYQQKPGQPPKLLIYLASNLESG
VPARFSGSGSGTDFTLNIHPVEEEDAATYYCQHSRDLPLTFGAGTKLELK
49 > 55D3F10
VH-CDR1:
(SEQ ID NO: 21)
DYAMA
VH-CDR2:
(SEQ ID NO: 58)
SIIYDGSSTDYRDSVK
VH-CDR3:
(SEQ ID NO: 91)
HGRVFGFGYFDY
VL-CDR1:
(SEQ ID NO: 111)
QASQDIGDWLA
VL-CDR2:
(SEQ ID NO: 132)
GADTLAD
VL-CDR3:
(SEQ ID NO: 158)
LQDSSAPYT
VH:
(SEQ ID NO: 208)
EVQLVESGGGLVQPGRSLKFSCVVSGFTFSDYAMAWVRQAPKKGLEWVASIIYDGSST
DYRDSVKGRFTISRDNAKSTLYLQMDGLRSEDTATYYCARHGRVFGFGYFDYWGQGV
MVTVSS
VL:
(SEQ ID NO: 245)
NIQMTQSPASLSASLEEVVTITCQASQDIGDWLAWFQQKPGKSPQLLIYGADTLADGVP
SRFSGSRSGTQFSLKISRLQVEDPGIYYCLQDSSAPYTFGAGTKLELK
50 > 98C7H11
VH-CDR1:
(SEQ ID NO: 21)
DYAMA
VH-CDR2:
(SEQ ID NO: 59)
TIIHDGSTTYYRDSVKG
VH-CDR3:
(SEQ ID NO: 91)
HGRVFGFGYFDY
VL-CDR1:
(SEQ ID NO: 111)
QASQDIGDWLA
VL-CDR2:
(SEQ ID NO: 130)
GATSLAD
VL-CDR3:
(SEQ ID NO: 159)
LQDYDPPYT
VH:
(SEQ ID NO: 209)
EVQLVESGGGLVQPGRSLKFSCAASGFTFSDYAMAWVRQAPKKGLEWVATIIHDGSTT
YYRDSVKGRFTISRDNAKTTLYLQMDILRSEDTATYYCVRHGRVFGFGYFDYWGQGV
MVTVSS
VL:
(SEQ ID NO: 246)
DIQMTQSPASLSASLEEIVTITCQASQDIGDWLAWFHQKPGKSPHLLIYGATSLADGVPS
RFSGSGSGTQFSLKISGLQVEDPGIYYCLQDYDPPYTFGAGTELELK
51 > 33A3E2
VH-CDR1:
(SEQ ID NO: 2)
DYGMV
VH-CDR2:
(SEQ ID NO: 47)
YIGSGRSTIYYADTVKG
VH-CDR3:
(SEQ ID NO: 61)
EDNGYSYAMDY
VL-CDR1:
(SEQ ID NO: 113)
LTSQTIGTWLA
VL-CDR2:
(SEQ ID NO: 115)
AATSLAD
VL-CDR3:
(SEQ ID NO: 134)
QQLYSIPLT
VH:
(SEQ ID NO: 188)
EVKLVESGGGLVQPGGSRKLSCAASGFTFSDYGMVWVRQAPGKGLEWVAYIGSGRSTI
YYADTVKGRFTISRDNPKNTLFLQMSSLRSEDTAMYYCAREDNGYSYAMDYWGQGTS
VTVSS
VL:
(SEQ ID NO: 247)
DIQMTQSPASQSASLGESVTITCLTSQTIGTWLAWYQQKPGKSPQLLIYAATSLADGVPS
RFSGSGSGTKFSFKISSLQAEDFVSYYCQQLYSIPLTFGAGTKLELK
52 > 85E10B9
VH-CDR1:
(SEQ ID NO: 249)
NHYLH
VH-CDR2:
(SEQ ID NO: 250)
WIGWIGPTNGDTSYAQKFKD
VH-CDR3:
(SEQ ID NO: 251)
EGYFTNYFDY
VL-CDR1:
(SEQ ID NO: 252)
KASQNVIKYLA
VL-CDR2:
(SEQ ID NO: 253)
NASSLQT
VL-CDR3:
(SEQ ID NO: 254)
LQYNSWWT
VH:
(SEQ ID NO: 255)
QVQLQQSGAELVKPAASVTLSCKASGYTFTNHYLHWVKQSPSQGLEWIGWIGPTNGDT
SYAQKFKDKATMTVDKSASTTYLQLSSLTSEDSAVYYCAREGYFTNYFDYWGQGVMV
TVSS
VL:
(SEQ ID NO: 256)
DIHMTQSPSFLSASVGDRVTLSCKASQNVIKYLAWYQQKLGEAPKLLIYNASSLQTGIPS
RFSGSGSGADFTLTISSLQPEDVATYFCLQYNSWWTFGGGTKLELK
Human IgG4 heavy chain constant region:
(SEQ ID NO: 257)
ASTKGPSVFPLAPCSRSTSESTAALGCLVKDYFPEPVTVSWNSGALTSGVHTFPAVLQSS
GLYSLSSVVTVPSSSLGTKTYTCNVDHKPSNTKVDKRVESKYGPPCPPCPAPEFLGGPSV
FLFPPKPKDTLMISRTPEVTCVVVDVSQEDPEVQFNWYVDGVEVHNAKTKPREEQFNST
YRVVSVLTVLHQDWLNGKEYKCKVSNKGLPSSIEKTISKAKGQPREPQVYTLPPSQEEM
TKNQVSLTCLVKGFYPSDIAVEWESNGQPENNYKTTPPVLDSDGSFFLYSRLTVDKSRW
QEGNVFSCSVMHEALHNHYTQKSLSLSLGK
Human kappa light chain constant region:
(SEQ ID NO: 258)
RTVAAPSVFIFPPSDEQLKSGTASVVCLLNNFYPREAKVQWKVDNALQSGNSQESVTEQ
DSKDSTYSLSSTLTLSKADYEKHKVYACEVTHQGLSSPVTKSFNRGEC
Human IgG1 heavy chain constant region:
(SEQ ID NO: 259)
ASTKGPSVFPLAPSSKSTSGGTAALGCLVKDYFPEPVTVSWNSGALTSGVHTFPAVLQSS
GLYSLSSVVTVPSSSLGTQTYICNVNHKPSNTKVDKKVEPKSCDKTHTCPPCPAPELLGG
PSVFLFPPKPKDTLMISRTPEVTCVVVDVSHEDPEVKFNWYVDGVEVHNAKTKPREEQY
NSTYRVVSVLTVLHQDWLNGKEYKCKVSNKALPAPIEKTISKAKGQPREPQVYTLPPSR
DELTKNQVSLTCLVKGFYPSDIAVEWESNGQPENNYKTTPPVLDSDGSFFLYSKLTVDK
SRWQQGNVFSCSVMHEALHNHYTQKSLSLSPGK

The following protein sequences are humanized mAb sequences:

>46H3H3-VH1
(SEQ ID NO: 260)
EVQLVESGGGLVQPGGSLRLSCAASGFTFSDYGMVWVRQAPGKGLEWVSY
IGSGRSTIYYADTVKGRFTISRDNAKNSLYLQMNSLRAEDTAVYYCARED
NGYSYAMDYWGQGTLVTVSS
>46H3H3-VH2
(SEQ ID NO: 261)
QVQLVESGGGLVKPGGSLRLSCAASGFTFSDYGMVWIRQAPGKGLEWVSY
IGSGRSTIYYADTVKGRFTISRDNAKNSLYLQMNSLRAEDTAVYYCARED
NGYSYAMDYWGQGTLVTVSS
>46H3H3-VH3
(SEQ ID NO: 262)
EVQLVESGGGLVKPGGSLRLSCAASGFTFSDYGMVWVRQAPGKGLEWVSY
IGSGRSTIYYADTVKGRFTISRDNAKNSLYLQMNSLRAEDTAVYYCARED
NGYSYAMDYWGQGTLVTVSS
>42C3A4-VH1
(SEQ ID NO: 263)
EVQLVESGGGLVQPGGSLKVSCAASGITFSNYWMDWVRQASGKGLEWVGE
IRLKSNNYATHYAESVKGRFTISRDDSKNTAYLQMNSLKTEDTAVYYCTR
GAPLFGGYYKGVYFDYWGQGTLVTVSS
>42C3A4-VH2
(SEQ ID NO: 264)
EVQLVESGGGLVQPGGSLRLSCAASGITFSNYWMDWVRQAPGKGLEWVAE
IRLKSNNYATHYAESVKGRFTISRDNAKNSLYLQMNSLRAEDTAVYYCAR
GAPLFGGYYKGVYFDYWGQGTLVTVSS
>42C3A4-VH3
(SEQ ID NO: 265)
EVQLVESGGGLVKPGGSLRLSCAASGITFSNYWMDWVRQAPGKGLEWVGE
IRLKSNNYATHYAESVKGRFTISRDDSKNTLYLQMNSLKTEDTAVYYCTT
GAPLFGGYYKGVYFDYWGQGTLVTVSS
>4D8F2-VH1
(SEQ ID NO: 266)
QVQLQESGPGLVKPSGTLSLTCAVSGYSITSDYAWNWVRQPPGKGLEWIG
YISYSGSTSYNPSLKSRVTISVDKSKNQFSLKLSSVTAADTAVYYCARGV
RGDMDYWGQGTLVTVSS
>4D8F2-VH1.1
(SEQ ID NO: 267)
QVQLQESGPGLVKPSGTLSLTCAVSGYSITSDYAWNWVRQPPGKGLEWIG
YISYSGSTSYNPSLKSRVTISVDKSKNQFSLKLSSVTAADTAVYYCARGV
RADMDYWGQGTLVTVSS
>4D8F2-VH1.2
(SEQ ID NO: 268)
QVQLQESGPGLVKPSGTLSLTCAVSGYSITSDYAWNWVRQPPGKGLEWIG
YISYSGSTSYNPSLKSRVTISVDKSKNQFSLKLSSVTAADTAVYYCARGV
RSDMDYWGQGTLVTVSS
>4D8F2-VH1.3
(SEQ ID NO: 269)
QVQLQESGPGLVKPSGTLSLTCAVSGYSITSDYAWNWVRQPPGKGLEWIG
YISYSGSTSYNPSLKSRVTISVDKSKNQFSLKLSSVTAADTAVYYCARGV
RGEMDYWGQGTLVTVSS
>4D8F2-VH2
(SEQ ID NO: 270)
QVQLQESGPGLVKPSQTLSLTCTVSGYSITSDYAWNWIRQPPGKGLEWIG
YISYSGSTSYNPSLKSRVTISVDTSKNQFSLKLSSVTAADTAVYYCARGV
RGDMDYWGQGTLVTVSS
>4D8F2-VH3
(SEQ ID NO: 271)
QVQLQQWGAGLLKPSETLSLTCAVYGYSITSDYAWNWIRQPPGKGLEWIG
YISYSGSTSYNPSLKSRVTISVDTSKNQFSLKLSSVTAADTAVYYCARGV
RGDMDYWGQGTLVTVSS
>42A2A6-VH1
(SEQ ID NO: 272)
QVQLQESGPGLVKPSQTLSLTCTVSGYSITSDYAWNWIRQPPGKGLEWIG
FINYSGTTSYNPSLKSRVTISVDTSKNQFSLKLSSVTAADTAVYYCARWG
YSDFLYYFDYWGQGTLVTVSS
>42A2A6-VH2
(SEQ ID NO: 273)
QVQLQESGPGLVKPSETLSLTCTVSGYSITSDYAWNWIRQPPGKGLEWIG
FINYSGTTSYNPSLKSRVTISVDTSKNQFSLKLSSVTAADTAVYYCARWG
YSDFLYYFDYWGQGTLVTVSS
>5A7B9-VH1
(SEQ ID NO: 274)
EVQLVESGSELKKPGASVKVSCKASGYTFTNYGVNWVRQAPGQGLEWMGW
INSYSGVPTYADDFKGRFVFSLDTSVSTAYLQISSLKAEDTAVYYCARGE
NNYYGGSYDWGQGTTVTVSS
>5A7B9-VH2
(SEQ ID NO: 275)
QVQVVQSGAEVKQPGASVKVSCKASGYTFTNYGVNWVRQAPGQGLEWMGW
INSYSGVPTYADDFKGRVTMTRDTSTSTAYMELRSLRSDDTAVYYCARGE
NNYYGGSYDWGQGTLVTVSS
>5A7B9-VH3
(SEQ ID NO: 276)
QVQLVQSGAEVKKPGASVKVSCKASGYTFTNYGVNWVRQAPGQRLEWMGW
INSYSGVPTYADDFKGRVTITRDTSASTAYMELSSLRSEDTAVYYCARGE
NNYYGGSYDWGQGTLVTVSS
>5A7B9-VH4
(SEQ ID NO: 277)
QVQLVQSGAEVKKPGASVKVSCKASGYTFTNYGVNWVRQATGQGLEWMGW
INSYSGVPTYADDFKGRVTMTRNTSISTAYMELSSLRSEDTAVYYCARGE
NNYYGGSYDWGQGTTVTVSS
>5A7B9-VH3.1
(SEQ ID NO: 278)
QVQLVQSGAEVKKPGASVKVSCKASGYTFTNYGVNWVRQAPGQRLEWMGW
INSYSGVPTYADDFKGRVTITRDTSASTAYLELSSLRSEDTAVYYCARGE
NNYYGGSYDWGQGTLVTVSS
>5A7B9-VH3.2
(SEQ ID NO: 279)
QVQLVQSGAEVKKPGASVKVSCKASGYTFTNYGVNWVRQAPGQRLEWMGW
INSYSGVPTYADDFKGRFTITRDTSASTAYMELSSLRSEDTAVYYCARGE
NNYYGGSYDWGQGTLVTVSS
>5A7B9-VH3.3
(SEQ ID NO: 280)
QVQLVQSGAEVKKPGASVKVSCKASGYTFTNYGVNWVRQAPGQRLEWMGW
INSYSGVPTYADDFKGRFTITRDTSASTAYLELSSLRSEDTAVYYCARGE
NNYYGGSYDWGQGTLVTVSS
>5A7B9-VH3.6
(SEQ ID NO: 281)
QVQLVQSGAEVKKPGASVKVSCKASGYTFTNYGVNWVRQAPGQRLEWMGW
INSYSGVPTYADDFKGRFTFTRDTSASTAYMELSSLRSEDTAVYYCARGE
NNYYGGSYDWGQGTLVTVSS
>5A7B9-VH3.7
(SEQ ID NO: 282)
QVQLVQSGAEVKKPGASVKVSCKASGYTFTNYGVNWVRQAPGQRLEWMGW
INSYSGVPTYADDFKGRFTFTLDTSASTAYMELSSLRSEDTAVYYCARGE
NNYYGGSYDWGQGTLVTVSS
>17F3E8-VH1.4
(SEQ ID NO: 283)
EVQLVESGAEVKKPGASVKVSCKASGYTFTNYYIHWVRQAPGQGLEWIGY
IHPNNGDTSYNQKFKDRATLTVDTSTSTVYMELSSLRSEDTAVYYCAVVS
YSHYVAGALDNWGQGTLVTVSS
>17F3E8-VH1.5
(SEQ ID NO: 284)
EVQLVESGAEVKKPGASVKVSCKASGYTFTNYYIHWVRQAPGQGIEWIGY
IHPNNGDTSYNQKFKDRATLTVDTSTSTVYMELSSLRSEDTAVYYCAVVS
YSHYVAGALDNWGQGTLVTVSS
>5F7D7-VH3
(SEQ ID NO: 285)
QVQLVQSGAEVKKPGASVKVSCKASGYTFTNYYMHWVRQAPGQRLEWIGY
IYPNNGDTSYNQKFKGRVTITVDTSASTAYMELSSLRSEDTAVYYCAVVS
YSNYVAGAMDYWGQGTLVTVSS
>5F7D7-VH5
(SEQ ID NO: 286)
QVQLVQSGAEVKKPGASVKVSCKASGYTFTNYYMHWVRQAHGQSLEWIGY
IYPNNGDTSYNQKFKGRVTITVDTSASTAYMELSSLRSEDTAVYYCAVVS
YSNYVAGAMDYWGQGTLVTVSS
>5F7D7-VH6
(SEQ ID NO: 287)
QVQLVQSGAEVKKPGASVKVSCKASGYTFTNYYMHWVRQAHGKSLEWIGY
IYPNNGDTSYNQKFKGRATITVDTSASTAYMELSSLRSEDTAVYYCAVVS
YSNYVAGAMDYWGQGTLVTVSS
>31E2D4-VH1
(SEQ ID NO: 288)
EVQLVESGSELKKPGASVKVSCKASGYTLTNYGMNWVRQAPGQGLEWMGW
INSYSGVPTYADDFKGRFVFSLDTSVSTAYLQISSLKAEDTAVYYCARGE
NNYYGGSYDWGQGTTVTVSS
>31E2D4-VH2
(SEQ ID NO: 289)
QVQVVQSGAEVKQPGASVKVSCKASGYTLTNYGMNWVRQAPGQGLEWMGW
INSYSGVPTYADDFKGRVTMTRDTSTSTAYMELRSLRSDDTAVYYCARGE
NNYYGGSYDWGQGTLVTVSS
>31E2D4-VH3
(SEQ ID NO: 290)
QVQLVQSGAEVKKPGASVKVSCKASGYTLTNYGMNWVRQAPGQRLEWMGW
INSYSGVPTYADDFKGRVTITRDTSASTAYMELSSLRSEDTAVYYCARGE
NNYYGGSYDWGQGTLVTVSS
>31E2D4-VH4
(SEQ ID NO: 291)
QVQLVQSGAEVKKPGASVKVSCKASGYTLTNYGMNWVRQATGQGLEWMGW
INSYSGVPTYADDFKGRVTMTRNTSISTAYMELSSLRSEDTAVYYCARGE
NNYYGGSYDWGQGTTVTVSS
>7F10B2-VH1
(SEQ ID NO: 292)
EVQLVESGAEVRKPGASVKVSCKASGYTFTDYNMDWVRQAPGQGLEWMGT
INPKNGETSDNQKFKARVTMTRDTSTSTVYMELSSLRSEDTAVYYCARNV
DYSNYLFFPMDYWGQGTLVTVSS
>7F10B2-VH2
(SEQ ID NO: 293)
QVQLVQSGAEVKKPGASVKVSCKASGYTFTDYNMDWVRQAPGQRLEWMGT
INPKNGETSDNQKFKARVTITRDTSASTAYMELSSLRSEDTAVYYCARNV
DYSNYLFFPMDYWGQGTLVTVSS
>7F10B2-VH3
(SEQ ID NO: 294)
QVQLVQSGAEVKKPGASVKVSCKASGYTFTDYNMDWVRQAPGQGLEWMGT
INPKNGETSDNQKFKARVTMTRDTSISTAYMELSRLRSDDTAVYYCARNV
DYSNYLFFPMDYWGQGTLVTVSS
>7F10B2-VH5
(SEQ ID NO: 295)
QVQLVQSGAEVKKPGASVKVSCKASGYTFTDYNMDWVRQAPGQGLEWMGT
INPKNGETSDNQKFKARVTVTTDTSTSTAYMELRSLRSDDTAVYYCARNV
DYSNYLFFPMDYWGQGTTVTVSS
>7F10B2-VH6
(SEQ ID NO: 296)
QVQLVQSGAEVKKPGSSVKVSCKASGYTFTDYNMDWVRQAPGQGLEWMGT
INPKNGETSDNQKFKARVTITADKSTSTAYMELSSLRSEDTAVYYCARNV
DYSNYLFFPMDYWGQGTTVTVSS
>46H3H3-VL1
(SEQ ID NO: 297)
DIQMTQSPSSLSASVGDRVTITCLASQTIGTWLAWYQQKPGKAPKLLIYA
ATSLADGVPSRFSGSGSGTDFTLTISSLQPEDFATYYCQQLYSTPLTFGG
GTKLEIK
>46H3H3-VL2
(SEQ ID NO: 298)
DIQMTQSPSSVSASVGDRVTITCLASQTIGTWLAWYQQKPGKAPKLLIYA
ATSLADGVPSRFSGSGSGTDFTLTISSLQPEDFATYYCQQLYSTPLTFGG
GTKVEIK
>46H3H3-VL3
(SEQ ID NO: 299)
DIQMTQSPSTLSASVGDRVTITCLASQTIGTWLAWYQQKPGKAPKLLIYA
ATSLADGVPSRFSGSGSGTEFTLTISSLQPDDFATYYCQQLYSTPLTFGG
GTKVEIK
>42C3A4-VL1
(SEQ ID NO: 300)
DIQMTQSPSSLSASVGDRVTITCQASQSVSNEVAWYQQKPGKAPKLLIYY
ASSRYTGVPSRFSGSGSGTDFTFTISSLQPEDIATYYCQQDYNSPYTFGQ
GTKLEIK
>42C3A4-VL2
(SEQ ID NO: 301)
DIQMTQSPSSLSASVGDRVTITCQASQSVSNEVAWYQQKPGKAPKLLIYY
ASSRYTGVPSRFSGSGSGTDFTLTISSLQPEDFATYYCQQDYNSPYTFGQ
GTKLEIK
>42C3A4-VL3
(SEQ ID NO: 302)
DIQMTQSPSSLSASVGDRVTITCQASQSVSNEVAWYQQKPGKVPKLLIYY
ASSRYTGVPSRFSGSGSGTDFTLTISSLQPEDVATYYCQQDYNSPYTFGQ
GTKLEIK
>4D8F2-VL1
(SEQ ID NO: 303)
DIQMTQSPSSLSASVGDRVTITCKASQSVSNDVAWYQQKPGKAPKLLIYY
ASNRYTGVPSRFSGSGSGTDFTLTISSLQPEDFATYYCQQDYSSPFTFGQ
GTKLEIK
>4D8F2-VL2
(SEQ ID NO: 304)
DIQLTQSPSSLSASVGDRVTITCKASQSVSNDVAWYQQKPGKAPKLLIYY
ASNRYTGVPSRFSGSGSGTDFTFTISSLQPEDFATYYCQQDYSSPFTFGQ
GTKVEIK
>4D8F2-VL3
(SEQ ID NO: 305)
EIVMTQSPATLSVSPGERATLSCKASQSVSNDVAWYQQKPGQAPRLLIYY
ASNRYTGIPARFSGSGSGTEFTLTINSLQSEDFAVYYCQQDYSSPFTFGQ
GTKLEIK
>42A2A6-VL1
(SEQ ID NO: 306)
EIVLTQSPATLSLSPGERATLSCRASSSVSYIHWYQQKPGQAPRLLIYAT
SNLASGIPARFSGSGSGTDFTLTISSLEPEDFAVYYCQQWSNNPWTFGQG
TKVEIK
>42A2A6-VL2
(SEQ ID NO: 307)
EIVLTQSPGTLSLSPGERATLSCRASSSVSYIHWYQQKPGQAPRLLIYAT
SNLASGIPDRFSGSGSGTDFTLTISRLEPEDFAVYYCQQWSNNPWTFGGG
TKVEIK
>42A2A6-VL3
(SEQ ID NO: 308)
DIQMTQSPSSLSASVGDRVTITCRASSSVSYIHWYQQKPGKAPKLLIYAT
SNLASGVPSRFSGSGSGTDFTLTISSLQPEDFATYYCQQWSNNPWTFGGG
TKLEIK
42A2A6-VL4
(SEQ ID NO: 309)
EIVMTQSPATLSVSPGERATLSCRASSSVSYIHWYQQKPGQAPRLLIYAT
SNLASGIPARFSGSGSGTEFTLTINSLQSEDFAVYYCQQWSNNPWTFGQG
TKLEIK
>5A7B9-VL1
(SEQ ID NO: 310)
DIQMTQSPSSLSASVGDRVTITCKASQSVSNDVAWYQQKPGKAPKLLIYY
ASNRYTGVPSRFSGSGSGTDFTLTISSLQPEDFATYYCQQDYSSPLTFGG
GTKLEIK
>5A7B9-VL2
(SEQ ID NO: 311)
DIQLTQSPSSLSASVGDRVTITCKASQSVSNDVAWYQQKPGKAPKLLIYY
ASNRYTGVPSRFSGSGSGTDFTFTISSLQPEDFATYYCQQDYSSPLTFGQ
GTKVEIK
>5A7B9-VL3
(SEQ ID NO: 312)
EIVMTQSPATLSVSPGERATLSCKASQSVSNDVAWYQQKPGQAPRLLIYY
ASNRYTGIPARFSGSGSGTEFTLTINSLQSEDFAVYYCQQDYSSPLTFGQ
GTKLEIK
>5A7B9-VL4
(SEQ ID NO: 313)
EIVLTQSPATLSLSPGERATLSCKASQSVSNDVAWYQQKPGQAPRLLIYY
ASNRYTGIPARFSGSGSGTDFTLTISSLEPEDFAVYYCQQDYSSPLTFGG
GTKVEIK
>5A7B9-VL1.1
(SEQ ID NO: 314)
DIQMTQSPSSLSASVGDRVTITCKASQSVSNDVAWYQQKPGKAPKLLIYY
ASNRYTGVPSRFSGSGSGTDFTLTISSLQPEDFATYFCQQDYSSPLTFGG
GTKLEIK
>5A7B9-VL1.2
(SEQ ID NO: 315)
DIQMTQSPSSLSASVGDRVTITCKASQSVSNDVAWYQQKPGKAPKLLIYY
ASNRYTGVPSRFSGSGSGTDFTFTISSLQPEDFATYFCQQDYSSPLTFGG
GTKLEIK
>5A7B9-VL1.3
(SEQ ID NO: 316)
DIQMTQSPSSLSASVGDRVTITCKASQSVSNDVAWYQQKPGKAPKLLIYY
ASNRYTGVPSRFSGSGSGTDFTFTISSLQPEDFATYYCQQDYSSPLTFGG
GTKLEIK
>5A7B9-VL2.1
(SEQ ID NO: 317)
DIQLTQSPSSLSASVGDRVTITCKASQSVSNDVAWYQQKPGKAPKLLIYY
ASNRYTGVPSRFSGSGSGTDFTFTISSLQPEDFATYFCQQDYSSPLTFGQ
GTKVEIK
>5F7D7-VL2.1
(SEQ ID NO: 318)
DIQLTQSPSSLSASVGDRVTITCKASQSVSNDVAWYQQKPGKAPKLLIYF
ASNRYTGVPSRFSGSGSGTDFTFTISSLQPEDFATYFCQQDYSSPYTFGQ
GTKVEIK
>5F7D7-VL2
(SEQ ID NO: 319)
DIQLTQSPSSLSASVGDRVTITCKASQSVSNDVAWYQQKPGKAPKLLIYF
ASNRYTGVPSRFSGSGSGTDFTFTISSLQPEDFATYYCQQDYSSPYTFGQ
GTKVEIK
>5F7D7-VL5
(SEQ ID NO: 320)
DIQMTQSPSSLSASVGDRVTITCKASQSVSNDVAWYQQKPGKAPKLLIYF
ASNRYTGVPSRFSGSGYGTDFTFTISSLQPEDIATYFCQQDYSSPYTFGG
GTKLEIK
>7F10B2-VL1
(SEQ ID NO: 321)
DIQMTQSPSSLSASVGDRVTITCKASQSVSNDVAWYQQKPGKAPKLLIYY
ASNRFTGVPSRFSGSGSGTDFTFTISSLQPEDIATYYCQQDYSSRTFGGG
TKVEIK
>7F10B2-VL2
(SEQ ID NO: 322)
EIVMTQSPATLSVSPGERATLSCKASQSVSNDVAWYQQKPGQAPRLLIYY
ASNRFTGIPARFSGSGSGTDFTLTISSLQSEDFAVYYCQQDYSSRTFGQG
TKVEIK
>7F10B2-VL3
(SEQ ID NO: 323)
DIQMTQSPSSLSASVGDRVTITCKASQSVSNDVAWYQQKPGKAPKLLIYY
ASNRFTGVPSRFSGSGSGTDFTLTISSLQPEDFATYYCQQDYSSRTFGGG
TKVEIK
>49E4C2-VL1
(SEQ ID NO: 324)
DIQMTQSPSSLSASVGDRVTITCKASQSVSNEVAWYQQKPGKAPKLLIYY
ASNRYTGVPSRFSGSGSGTDFTFTISSLQPEDIATYYCQQDYNSPYTFGG
GTKLEIK
>49E4C2-VH1
(SEQ ID NO: 325)
EVQLVESGGGLVQPGGSLKVSCAASGITFSNYWMDWVRQASGKGLEWVGE
IRLKSNNYATHYAESVKGRFTISRDDSKNTAYLQMNSLKTEDTAVYYCTR
GAPLFDGYYKGVYFDYWGQGTLVTVSS
>53E8B2-VH1
(SEQ ID NO: 326)
QVQLVESGGGVVQPGRSLRLSCAASGFTFSDYAMAWVRQAPGKGLEWVAT
IIYDGSSTYYRDSVKGRFTISRDNSKNTLYLQMNSLRAEDTAVYYCARHG
RVFGFGYFDYWGQGTLVTVSS
>53E8B2-VH2
(SEQ ID NO: 327)
EVQLVESGGGLVQPGGSLRLSCAASGFTFSDYAMAWVRQAPGKGLEWVST
IIYDGSSTYYRDSVKGRFTISRDNSKNTLYLQMNSLRAEDTAVYYCAKHG
RVFGFGYFDYWGQGTLVTVSS
>53E8B2-VH3
(SEQ ID NO: 328)
EVQLVESGGGLVQPGGSLRLSCAASGFTFSDYAMAWVRQAPGKGLEWVAT
IIYDGSSTYYRDSVKGRFTISRDNAKNSLYLQMNSLRAEDTAVYYCARHG
RVFGFGYFDYWGQGTLVTVSS
>53E8B2-VL3
(SEQ ID NO: 336)
DIQMTQSPSTLSASVGDRVTITCQASQDIGDWVAWYQQKPGKAPKVLIYG
ADRLADGVPSRFSGSGSGTEFTLTISSLQPDDFATYYCLQDYSPPFTFGP
GTKLEIK
>53E8B2-VL6
(SEQ ID NO: 337)
DIQMTQSPSSLSASVGDRVTITCQASQDIGDWVAWYQQKPGKAPKRLIYG
ADRLADGVPSRFSGSGSGTEFTLTISSLQPEDFATYYCLQDYSPPFTFGQ
GTKVEIK
>53E8B2-VL2
(SEQ ID NO: 338)
DIQMTQSPSSVSASVGDRVTITCQASQDIGDWVAWYQQKPGRAPKLLIYG
ADRLADGVPSRFSGSGSGTDFTLTISSLQPEDFATYYCLQDYSPPFTFGQ
GTKLEIK
>85E10B9-VH1
(SEQ ID NO: 329)
QVQLVQSGAEVKKPGASVKVSCKASGYTFTNHYLHWVRQAPGQGLEWMGW
IGPTNGDTSYAQKFKDRVTMTRDTSISTAYMELSRLRSDDTAVYYCAREG
YFTNYFDYWGQGTLVTVSS
>85E10B9-VH3
(SEQ ID NO: 330)
QVQLVQSGAEVKKPGASVKVSCKASGYTFTNHYLHWVRQAPGQRLEWMGW
IGPTNGDTSYAQKFKDRVTITRDTSASTAYMELSSLRSEDTAVYYCAREG
YFTNYFDYWGQGTLVTVSS
>85E10B9-VL1
(SEQ ID NO: 339)
DIQMTQSPSSLSASVGDRVTITCKASQNVIKYLAWYQQKPGKVPKLLIYN
ASSLQTGVPSRFSGSGSGTDFTLTISSLQPEDVATYYCLQYNSWWTFGGG
TKVEIK
>85E10B9-VL2
(SEQ ID NO: 340)
DIQLTQSPSFLSASVGDRVTITCKASQNVIKYLAWYQQKPGKAPKLLIYN
ASSLQTGVPSRFSGSGSGTEFTLTISSLQPEDFATYYCLQYNSWWTFGGG
TKVEIK
>85E10B9-VL6
(SEQ ID NO: 341)
DIQMTQSPSSLSASVGDRVTITCKASQNVIKYLAWYQQKPGKAPKRLIYN
ASSLQTGVPSRFSGSGSGTEFTLTISSLQPDDFATYYCLQYNSWWTFGQG
TKVEIK
>60C4B9-VH1
(SEQ ID NO: 331)
QVQLVESGGGVVQPGRSLRLSCAASGFTFSDYAMAWVRQAPGKGLEWVAT
IIHNGGTTFYRDSVKGRFTISRDNSKNTLYLQMNSLRAEDTAVYYCARHG
RVFGFGYFDYWGQGTLVTVSS
>60C4B9-VL6
(SEQ ID NO: 342)
DIQMTQSPSSLSASVGDRVTITCQASQDIGDWLAWYQQKPGKAPKRLIYG
ATSLADGVPSRFSGSGSGTEFTLTISSLQPEDFATYYCLQDSDPPYTFGQ
GTKVEIK
>98C7H11-VH3
(SEQ ID NO: 332)
EVQLVESGGGLVQPGGSLRLSCAASGFTFSDYAMAWVRQAPGKGLEWVAT
IIHDGSTTYYRDSVKGRFTISRDNAKNSLYLQMNSLRAEDTAVYYCARHG
RVFGFGYFDYWGQGTLVTVSS
>98C7H11-VL2
(SEQ ID NO: 343)
DIQMTQSPSTLSASVGDRVTITCQASQDIGDWLAWYQQKPGKAPKLLIYG
ATSLADGVPSRFSGSGSGTEFTLTISSLQPDDFATYYCLQDYDPPYTFGQ
GTKLEIK
>55D3F10-VH1
(SEQ ID NO: 333)
QVQLVESGGGVVQPGRSLRLSCAASGFTFSDYAMAWVRQAPGKGLEWVAS
IIYDGSSTDYRDSVKGRFTISRDNSKNTLYLQMNSLRAEDTAVYYCARHG
RVFGFGYFDYWGQGTLVTVSS
>55D3F10-VH2
(SEQ ID NO: 334)
EVQLVESGGGLVQPGGSLRLSCAASGFTFSDYAMAWVRQAPGKGLEWVSS
IIYDGSSTDYRDSVKGRFTISRDNSKNTLYLQMNSLRAEDTAVYYCAKHG
RVFGFGYFDYWGQGTLVTVSS
>55D3F10-VL2
(SEQ ID NO: 344)
DIQMTQSPSSLSASVGDRVTITCQASQDIGDWLAWYQQKPGKAPKLLIYG
ADTLADGVPSRFSGSGSGTDFTLTISSLQPEDFATYYCLQDSSAPYTFGQ
GTKVEIK
>55D3F10-VL6
(SEQ ID NO: 345)
DIQMTQSPSSLSASVGDRVTITCQASQDIGDWLAWYQQKPGKAPKRLIYG
ADTLADGVPSRFSGSGSGTEFTLTISSLQPEDFATYYCLQDSSAPYTFGQ
GTKVEIK
>73H11G10-VH1
(SEQ ID NO: 335)
QVQLVESGGGVVQPGRSLRLSCAASGFTFSDYAMAWVRQAPGKGLEWVAT
IIYDGSSTVTVSS
>73H11G10-VL6
73H11G10-VL6 and 53E8B2-VL6 share the
same sequence

Having thus described in detail preferred embodiments of the present invention, it is to be understood that the invention defined by the above paragraphs is not to be limited to particular details set forth in the above description as many apparent variations thereof are possible without departing from the spirit or scope of the present invention.

1. (canceled)

2. An isolated monoclonal antibody, or an antigen-binding portion thereof that specifically binds to angiopoietin 2 comprising a VH CDR1, a VH CDR2, a VH CDR3, a VL CDR1, a VL CDR2 and a VL CDR3, wherein the VH CDR1, VH CDR2, VH CDR3, VL CDR1, VL CDR2 and VL CDR3 comprise the amino acid sequences of (1) SEQ ID NOs: 8, 29, 65, 92, 116 and 136, respectively; (2) SEQ ID NOs: 2, 24, 61, 93, 115 and 134, respectively; (3) SEQ ID NOs: 3, 25, 62, 92, 114 and 133, respectively; (4) SEQ ID NOs: 3, 26, 62, 92, 114 and 133, respectively; (5) SEQ ID NOs: 4, 27, 63, 92, 116 and 135, respectively; (6) SEQ ID NOs: 5, 28, 64, 92, 114 and 133, respectively; (7) SEQ ID NOs: 6, 29, 65, 92, 116 and 136, respectively; (8) SEQ ID NOs: 1, 25, 62, 92, 114 and 133, respectively; (9) SEQ ID NOs: 7, 30, 66, 92, 117 and 137, respectively; (10) SEQ ID NOs: 1, 23, 60, 92, 114 and 133, respectively; (11) SEQ ID NOs: 4, 31, 63, 92, 116 and 135, respectively; (12) SEQ ID NOs: 5, 32, 67, 92, 114 and 133, respectively; (13) SEQ ID NOs: 3, 33, 68, 92, 114 and 133, respectively; (14) SEQ ID NOs: 9, 34, 69, 94, 118 and 138, respectively; (15) SEQ ID NOs: 1, 35, 62, 92, 114 and 133, respectively; (16) SEQ ID NOs: 4, 27, 70, 95, 119 and 139, respectively; (17) SEQ ID NOs: 4, 27, 71, 96, 116 and 136, respectively; (18) SEQ ID NOs: 10, 36, 72, 97, 120 and 140, respectively; (19) SEQ ID NOs: 4, 37, 73, 98, 121 and 141, respectively; (20) SEQ ID NOs: 11, 38, 74, 99, 122 and 142, respectively; (21) SEQ ID NOs: 12, 39, 75, 100, 123 and 143, respectively; (22) SEQ ID NOs: 13, 40, 76, 101, 124 and 144, respectively; (23) SEQ ID NOs: 14, 41, 77, 97, 120 and 145, respectively; (24) SEQ ID NOs: 5, 42, 64, 92, 114 and 133, respectively; (25) SEQ ID NOs: 5, 43, 78, 102, 115 and 146, respectively; (26) SEQ ID NOs: 15, 44, 79, 103, 115 and 147, respectively; (27) SEQ ID NOs: 8, 45, 80, 104, 125 and 148, respectively; (28) SEQ ID NOs: 16, 46, 81, 104, 125 and 148, respectively; (29) SEQ ID NOs: 2, 47, 61, 93, 115 and 149, respectively; (30) SEQ ID NOs: 17, 48, 82, 105, 126 and 150, respectively; (31) SEQ ID NOs: 11, 38, 83, 106, 116 and 142, respectively; (32) SEQ ID NOs: 10, 49, 84, 97, 120 and 151, respectively; (33) SEQ ID NOs: 2, 50, 61, 93, 115 and 134, respectively; (34) SEQ ID NOs: 10, 36, 85, 97, 120 and 152, respectively; (35) SEQ ID NOs: 5, 32, 86, 92, 114 and 133, respectively; (36) SEQ ID NOs: 18, 51, 87, 103, 115 and 147, respectively; (37) SEQ ID NOs: 2, 52, 88, 103, 115 and 149, respectively; (38) SEQ ID NOs: 19, 53, 89, 107, 127 and 153, respectively; (39) SEQ ID NOs: 5, 32, 64, 92, 114 and 133, respectively; (40) SEQ ID NOs: 20, 54, 90, 108, 128 and 154, respectively; (41) SEQ ID NOs: 2, 47, 61, 93, 115 and 134, respectively; (42) SEQ ID NOs: 4, 27, 71, 109, 116 and 136, respectively; (43) SEQ ID NOs: 21, 55, 91, 110, 129 and 155, respectively; (44) SEQ ID NOs: 21, 56, 91, 111, 130 and 156, respectively; (45) SEQ ID NOs: 21, 55, 346, 110, 129 and 155, respectively; (46) SEQ ID NOs: 22, 57, 347, 112, 131 and 157, respectively; (47) SEQ ID NOs: 21, 58, 91, 111, 132 and 158, respectively; (48) SEQ ID NOs: 21, 59, 91, 111, 130 and 159, respectively; (49) SEQ ID NOs: 2, 47, 61, 113, 115 and 134, respectively; or (50) SEQ ID NOs: 249, 250, 251, 252, 253 and 254, respectively.

3. The isolated monoclonal antibody, or the antigen-binding portion thereof, of claim 2, wherein the heavy chain variable region comprises an amino acid sequence having at least 85%, 86%, 87%, 88%, 89%, 90%, 91%, 92%, 93%, 94%, 95%, 96%, 97%, 98%, 99% or 100% identity to any one of SEQ ID NOs: 160 to 209, 255, 325, 260 to 296 and 326 to 335; and wherein the light chain variable region comprises an amino acid sequence having at least 85%, 86%, 87%, 88%, 89%, 90%, 91%, 92%, 93%, 94%, 95%, 96%, 97%, 98%, 99% or 100% identity to any one of SEQ ID NOs: 210 to 247, 256, 297 to 324 and 336 to 345.

4. (canceled)

5. The isolated monoclonal antibody, or the antigen-binding portion thereof, of claim 2, wherein the heavy chain variable region and the light chain variable region comprise amino acid sequences having at least 85%, 86%, 87%, 88%, 89%, 90%, 91%, 92%, 93%, 94%, 95%, 96%, 97%, 98%, 99% or 100% identity to (1) SEQ ID NOs: 160 and 210, respectively; (2) SEQ ID NOs: 161 and 211, respectively; (3) SEQ ID NOs: 162 and 212, respectively; (4) SEQ ID NOs: 163 and 210, respectively; (5) SEQ ID NOs: 164 and 213, respectively; (6) SEQ ID NOs: 165 and 210, respectively; (7) SEQ ID NOs: 166 and 214, respectively; (8) SEQ ID NOs: 167 and 215, respectively; (9) SEQ ID NOs: 168 and 216, respectively; (10) SEQ ID NOs: 169 and 217, respectively; (11) SEQ ID NOs: 170 and 218, respectively; (12) SEQ ID NOs: 171 and 210, respectively; (13) SEQ ID NOs: 172 and 210, respectively; (14) SEQ ID NOs: 173 and 219, respectively; (15) SEQ ID NOs: 174 and 210, respectively; (16) SEQ ID NOs: 175 and 220, respectively; (17) SEQ ID NOs: 176 and 221, respectively; (18) SEQ ID NOs: 177 and 222, respectively; (19) SEQ ID NOs: 178 and 223, respectively; (20) SEQ ID NOs: 179 and 224, respectively; (21) SEQ ID NOs: 180 and 225, respectively; (22) SEQ ID NOs: 181 and 226, respectively; (23) SEQ ID NOs: 182 and 227, respectively; (24) SEQ ID NOs: 183 and 210, respectively; (25) SEQ ID NOs: 184 and 228, respectively; (26) SEQ ID NOs: 185 and 229, respectively; (27) SEQ ID NOs: 186 and 230, respectively; (28) SEQ ID NOs: 187 and 230, respectively; (29) SEQ ID NOs: 188 and 231, respectively; (30) SEQ ID NOs: 189 and 232, respectively; (31) SEQ ID NOs: 190 and 233, respectively; (32) SEQ ID NOs: 191 and 234, respectively; (33) SEQ ID NOs: 192 and 235, respectively; (34) SEQ ID NOs: 193 and 236, respectively; (35) SEQ ID NOs: 194 and 212, respectively; (36) SEQ ID NOs: 195 and 229, respectively; (37) SEQ ID NOs: 196 and 237, respectively; (38) SEQ ID NOs: 197 and 248, respectively; (39) SEQ ID NOs: 198 and 215, respectively; (40) SEQ ID NOs: 199 and 238, respectively; (41) SEQ ID NOs: 200 and 239, respectively; (42) SEQ ID NOs: 201 and 239, respectively; (43) SEQ ID NOs: 202 and 240, respectively; (44) SEQ ID NOs: 203 and 241, respectively; (45) SEQ ID NOs: 204 and 242, respectively; (46) SEQ ID NOs: 205 and 243, respectively; (47) SEQ ID NOs: 206 and 242, respectively; (48) SEQ ID NOs: 207 and 244, respectively; (49) SEQ ID NOs: 208 and 245, respectively; (50) SEQ ID NOs: 209 and 246, respectively; (51) SEQ ID NOs: 188 and 247, respectively; or (52) SEQ ID NOs: 255 and 256, respectively.

6. The isolated monoclonal antibody, or the antigen-binding portion thereof, of claim 2, wherein the heavy chain variable region and the light chain variable region comprise amino acid sequences having at least 85%, 86%, 87%, 88%, 89%, 90%, 91%, 92%, 93%, 94%, 95%, 96%, 97%, 98%, 99% or 100% identity to (1) SEQ ID NOs: 291 and 310, respectively; (2) SEQ ID NOs: 260 and 298, respectively; (3) SEQ ID NOs: 260 and 299, respectively; (4) SEQ ID NOs: 261 and 297, respectively; (5) SEQ ID NOs: 261 and 298, respectively; (6) SEQ ID NOs: 261 and 299, respectively; (7) SEQ ID NOs: 262 and 297, respectively; (8) SEQ ID NOs: 262 and 298, respectively; (9) SEQ ID NOs: 262 and 299, respectively; (10) SEQ ID NOs: 263 and 300, respectively; (11) SEQ ID NOs: 263 and 301, respectively; (12) SEQ ID NOs: 263 and 302, respectively; (13) SEQ ID NOs: 264 and 300, respectively; (14) SEQ ID NOs: 264 and 301, respectively; (15) SEQ ID NOs: 264 and 302, respectively; (16) SEQ ID NOs: 265 and 300, respectively; (17) SEQ ID NOs: 266 and 303, respectively; (18) SEQ ID NOs: 267 and 303, respectively; (19) SEQ ID NOs: 268 and 303, respectively; (20) SEQ ID NOs: 269 and 303, respectively; (21) SEQ ID NOs: 270 and 304, respectively; (22) SEQ ID NOs: 271 and 305, respectively; (23) SEQ ID NOs: 325 and 324, respectively; (24) SEQ ID NOs: 272 and 306, respectively; (25) SEQ ID NOs: 272 and 307, respectively; (26) SEQ ID NOs: 272 and 308, respectively; (27) SEQ ID NOs: 272 and 309, respectively; (28) SEQ ID NOs: 273 and 306, respectively; (29) SEQ ID NOs: 273 and 307, respectively; (30) SEQ ID NOs: 273 and 308, respectively; (31) SEQ ID NOs: 273 and 309, respectively; (32) SEQ ID NOs: 274 and 310, respectively; (33) SEQ ID NOs: 274 and 311, respectively; (34) SEQ ID NOs: 275 and 310, respectively; (35) SEQ ID NOs: 275 and 311, respectively; (36) SEQ ID NOs: 275 and 312, respectively; (37) SEQ ID NOs: 276 and 310, respectively; (38) SEQ ID NOs: 276 and 311, respectively; (39) SEQ ID NOs: 276 and 312, respectively; (40) SEQ ID NOs: 277 and 313, respectively; (41) SEQ ID NOs: 276 and 314, respectively; (42) SEQ ID NOs: 276 and 315, respectively; (43) SEQ ID NOs: 276 and 316, respectively; (44) SEQ ID NOs: 276 and 317, respectively; (45) SEQ ID NOs: 281 and 310, respectively; (46) SEQ ID NOs: 282 and 310, respectively; (47) SEQ ID NOs: 278 and 310, respectively; (48) SEQ ID NOs: 279 and 310, respectively; (49) SEQ ID NOs: 280 and 310, respectively; (50) SEQ ID NOs: 280 and 311, respectively; (51) SEQ ID NOs: 283 and 319, respectively; (52) SEQ ID NOs: 284 and 319, respectively; (53) SEQ ID NOs: 283 and 318, respectively; (54) SEQ ID NOs: 284 and 318, respectively; (55) SEQ ID NOs: 288 and 310, respectively; (56) SEQ ID NOs: 290 and 310, respectively; (57) SEQ ID NOs:260 and 297, respectively; (58) SEQ ID NOs: 288 and 311, respectively; (59) SEQ ID NOs: 291 and 311, respectively; (60) SEQ ID NOs: 288 and 312, respectively; (61) SEQ ID NOs: 285 and 320, respectively; (62) SEQ ID NOs: 286 and 320, respectively; (63) SEQ ID NOs: 287 and 320, respectively; (64) SEQ ID NOs: 292 and 323, respectively; (65) SEQ ID NOs: 293 and 321, respectively; (66) SEQ ID NOs: 293 and 322, respectively; (67) SEQ ID NOs: 293 and 323, respectively; (68) SEQ ID NOs: 294 and 321, respectively; (69) SEQ ID NOs: 294 and 322, respectively; (70) SEQ ID NOs: 294 and 323, respectively; (71) SEQ ID NOs: 295 and 321, respectively; (72) SEQ ID NOs: 296 and 322, respectively; (73) SEQ ID NOs: 326 and 336, respectively; (74) SEQ ID NOs: 326 and 337, respectively; (75) SEQ ID NOs: 327 and 338, respectively; (76) SEQ ID NOs: 328 and 338, respectively; (77) SEQ ID NOs: 329 and 341, respectively; (78) SEQ ID NOs: 330 and 339, respectively; (79) SEQ ID NOs: 330 and 340, respectively; (80) SEQ ID NOs: 327 and 337, respectively; (81) SEQ ID NOs: 331 and 342, respectively; (82) SEQ ID NOs: 335 and 337, respectively; (83) SEQ ID NOs: 332 and 343, respectively; (84) SEQ ID NOs: 333 and 345, respectively; (85) SEQ ID NOs: 334 and 345, respectively; or (86) SEQ ID NOs: 334 and 344, respectively.

7. The isolated monoclonal antibody, or the antigen-binding portion thereof, of claim 2, comprising a heavy chain constant region and/or a light chain constant region, wherein the heavy chain const region is an IgG, IgA, IgD, IgE and IgM constant region with reduced or no FcR binding affinity.

8. (canceled)

9. The isolated monoclonal antibody, or the antigen-binding portion thereof, of claim 7, wherein the heavy chain constant region is an IgG1 or an IgG4 constant region.

10. The isolated monoclonal antibody, or the antigen-binding portion thereof, of claim 7, wherein the light chain constant region is kappa constant region.

11. The isolated monoclonal antibody, or the antigen-binding portion thereof, of claim 2, which is a mouse, chimeric or humanized antibody or antigen-binding portion thereof.

12. The isolated monoclonal antibody, or the antigen-binding portion thereof, of claim 2, which (a) binds human Ang2 protein or a fragment thereof, (b) binds monkey Ang2 protein or a fragment thereof, (c) binds mouse Ang2 protein or a fragment thereof, (d) inhibits Ang2 binding to Tie2, (e) inhibits Ang2 binding to integrins, and/or (f) activates Tie2 signaling.

13. A bispecific molecule comprising the antibody, or the antigen-binding portion thereof, of claim 2.

14. A nucleic acid molecule encoding the antibody, or the antigen-binding portion thereof, of claim 2.

15. An expression vector containing the nucleic acid molecule of claim 14.

16. A host cell containing the expression vector of claim 15.

17. A composition comprising the antibody, or the antigen-binding portion thereof, of claim 2.

18. The composition of claim 17, further comprising an anti-tumor agent, an anti-inflammatory agent, or an anti-infectious agent.

19. A method for treating an Ang2 related disease in a subject in need thereof, comprising administering to the subject a therapeutically effective amount of the composition of claim 17.

20. The method of claim 19, wherein the disease is cancer, an inflammatory disease or an infectious disease.

21.-22. (canceled)

23. The method of claim 20, wherein the inflammatory disease is sepsis, macular edema, diabetic retinopathy or age-related macular degeneration.

24.-26. (canceled)

27. A method for stabilizing the vasculature or reducing vascular inflammation in a subject in need thereof, comprising administering to the subject a therapeutically effective amount of the composition of claim 17.


SEQUENCE LISTING (347 sequences)

SEQ ID NO: 1 - protein, 5 aa, Artificial Sequence
  43F1C10-2 VH-CDR1
NYYMH

SEQ ID NO: 2 - protein, 5 aa, Artificial Sequence
  49E9G10 VH-CDR1
DYGMV

SEQ ID NO: 3 - protein, 5 aa, Artificial Sequence
  50B9C10 VH-CDR1
NYYIH

SEQ ID NO: 4 - protein, 6 aa, Artificial Sequence
  4D8F2 VH-CDR1
SDYAWN

SEQ ID NO: 5 - protein, 5 aa, Artificial Sequence
  50H8G3 VH-CDR1
DFYME

SEQ ID NO: 6 - protein, 5 aa, Artificial Sequence
  5A7B9 VH-CDR1
NYGVN

SEQ ID NO: 7 - protein, 5 aa, Artificial Sequence
  7F10B2 VH-CDR1
DYNMD

SEQ ID NO: 8 - protein, 5 aa, Artificial Sequence
  31E2D4 VH-CDR1
NYGMN

SEQ ID NO: 9 - protein, 5 aa, Artificial Sequence
  82B10C1 VH-CDR1
DYIIH

SEQ ID NO: 10 - protein, 5 aa, Artificial Sequence
  41H8A8 VH-CDR1
SNWMH

SEQ ID NO: 11 - protein, 5 aa, Artificial Sequence
  42C3A4 VH-CDR1
NYWMD

SEQ ID NO: 12 - protein, 5 aa, Artificial Sequence
  43B2A12 VH-CDR1
GYWMH

SEQ ID NO: 13 - protein, 5 aa, Artificial Sequence
  43C4A11 VH-CDR1
SYWMH

SEQ ID NO: 14 - protein, 5 aa, Artificial Sequence
  43D3A9 VH-CDR1
SNWIH

SEQ ID NO: 15 - protein, 5 aa, Artificial Sequence
  45C5A1 VH-CDR1
NYTMS

SEQ ID NO: 16 - protein, 5 aa, Artificial Sequence
  45C9F9-2 VH-CDR1
SYGVH

SEQ ID NO: 17 - protein, 5 aa, Artificial Sequence
  49C3B8 VH-CDR1
SDNWN

SEQ ID NO: 18 - protein, 5 aa, Artificial Sequence
  50C9C2 VH-CDR1
NYMMS

SEQ ID NO: 19 - protein, 5 aa, Artificial Sequence
  54E4A12 VH-CDR1
TYNVH

SEQ ID NO: 20 - protein, 5 aa, Artificial Sequence
  76B2E6 VH-CDR1
RFYIY

SEQ ID NO: 21 - protein, 5 aa, Artificial Sequence
  53E8B2 VH-CDR1
DYAMA

SEQ ID NO: 22 - protein, 5 aa, Artificial Sequence
  15B4C6 VH-CDR1
GYGVN

SEQ ID NO: 23 - protein, 17 aa, Artificial Sequence
  43F1C10-2 VH-CDR2
YIHPNNGDTSYNQKFKG

SEQ ID NO: 24 - protein, 17 aa, Artificial Sequence
  49E9G10 VH-CDR2
YIGSGRNTIYYVDTVKG

SEQ ID NO: 25 - protein, 17 aa, Artificial Sequence
  50B9C10 VH-CDR2
YIYPNNGDTSYNQKFKG

SEQ ID NO: 26 - protein, 17 aa, Artificial Sequence
  46F3H4 VH-CDR2
YIYPNNGDTTYNQKFKG

SEQ ID NO: 27 - protein, 16 aa, Artificial Sequence
  4D8F2 VH-CDR2
YISYSGSTSYNPSLKS

SEQ ID NO: 28 - protein, 19 aa, Artificial Sequence
  50H8G3 VH-CDR2
ASRNKAKDYTTEYSASVKG

SEQ ID NO: 29 - protein, 17 aa, Artificial Sequence
  5A7B9 VH-CDR2
WINSYSGVPTYADDFKG

SEQ ID NO: 30 - protein, 17 aa, Artificial Sequence
  7F10B2 VH-CDR2
TINPKNGETSDNQKFKA

SEQ ID NO: 31 - protein, 16 aa, Artificial Sequence
  15H10D12 VH-CDR2
YINYSGSTDYNPSLKS

SEQ ID NO: 32 - protein, 19 aa, Artificial Sequence
  25C5D6F3 VH-CDR2
ASRNKANDYTTEYSASVKG

SEQ ID NO: 33 - protein, 17 aa, Artificial Sequence
  17F3E8 VH-CDR2
YIHPNNGDTSYNQKFKD

SEQ ID NO: 34 - protein, 17 aa, Artificial Sequence
  82B10C1 VH-CDR2
YINPFSGGTNYNEKFKS

SEQ ID NO: 35 - protein, 17 aa, Artificial Sequence
  50H8E5C5 VH-CDR2
YIYPNNGDTNYNQKFKG

SEQ ID NO: 36 - protein, 17 aa, Artificial Sequence
  41H8A8 VH-CDR2
AIYPGNSDTSYNQKFKD

SEQ ID NO: 37 - protein, 16 aa, Artificial Sequence
  42A2A6 VH-CDR2
FINYSGTTSYNPSLKS

SEQ ID NO: 38 - protein, 19 aa, Artificial Sequence
  42C3A4 VH-CDR2
EIRLKSNNYATHYAESVKG

SEQ ID NO: 39 - protein, 17 aa, Artificial Sequence
  43B2A12 VH-CDR2
EIDPSDSFTYNNQKFKG

SEQ ID NO: 40 - protein, 17 aa, Artificial Sequence
  43C4A11 VH-CDR2
EIDPSDSYSYYNQKFKG

SEQ ID NO: 41 - protein, 17 aa, Artificial Sequence
  43D3A9 VH-CDR2
AIYPGNNDTSYNQKFKD

SEQ ID NO: 42 - protein, 19 aa, Artificial Sequence
  43F1C10 VH-CDR2
ASRDKANDYTTEYSASVKG

SEQ ID NO: 43 - protein, 19 aa, Artificial Sequence
  43G5G3 VH-CDR2
SSRNKANDYTTEYNASVKG

SEQ ID NO: 44 - protein, 17 aa, Artificial Sequence
  45C5A1 VH-CDR2
EISNSDGSAYYPDTVKG

SEQ ID NO: 45 - protein, 17 aa, Artificial Sequence
  45C9F9 VH-CDR2
WISTYSGVPTYADDFKG

SEQ ID NO: 46 - protein, 16 aa, Artificial Sequence
  45C9F9-2 VH-CDR2
VIWAGGSTNYNSALMS

SEQ ID NO: 47 - protein, 17 aa, Artificial Sequence
  46H3H3 VH-CDR2
YIGSGRSTIYYADTVKG

SEQ ID NO: 48 - protein, 16 aa, Artificial Sequence
  49C3B8 VH-CDR2
YISYSGSSYYNPSLKS

SEQ ID NO: 49 - protein, 17 aa, Artificial Sequence
  49E4F1 VH-CDR2
AIYPGNSDTDYNQKFKD

SEQ ID NO: 50 - protein, 17 aa, Artificial Sequence
  49G4D6 VH-CDR2
YIGSGRNTIYYADTVKG

SEQ ID NO: 51 - protein, 17 aa, Artificial Sequence
  50C9C2 VH-CDR2
EISNSDGSTYYPDTVKG

SEQ ID NO: 52 - protein, 17 aa, Artificial Sequence
  50F11D12 VH-CDR2
YISGGSKTVYYADTVKG

SEQ ID NO: 53 - protein, 16 aa, Artificial Sequence
  54E4A12 VH-CDR2
VIWNTGGTRYNSTLKS

SEQ ID NO: 54 - protein, 17 aa, Artificial Sequence
  76B2E6 VH-CDR2
YIYPGNGDTDYSEKFKG

SEQ ID NO: 55 - protein, 17 aa, Artificial Sequence
  53E8B2 VH-CDR2
TIIYDGSSTYYRDSVKG

SEQ ID NO: 56 - protein, 17 aa, Artificial Sequence
  60C4B9 VH-CDR2
TIIHNGGTTFYRDSVKG

SEQ ID NO: 57 - protein, 16 aa, Artificial Sequence
  15B4C6 VH-CDR2
LIWNNGSTDYNSALKS

SEQ ID NO: 58 - protein, 16 aa, Artificial Sequence
  55D3F10 VH-CDR2
SIIYDGSSTDYRDSVK

SEQ ID NO: 59 - protein, 17 aa, Artificial Sequence
  98C7H11 VH-CDR2
TIIHDGSTTYYRDSVKG

SEQ ID NO: 60 - protein, 13 aa, Artificial Sequence
  43F1C10-2 VH-CDR3
VSYSHYVAGAMDY

SEQ ID NO: 61 - protein, 11 aa, Artificial Sequence
  49E9G10 VH-CDR3
EDNGYSYAMDY

SEQ ID NO: 62 - protein, 13 aa, Artificial Sequence
  50B9C10VH-CDR3
VSYSNYVAGAMDY

SEQ ID NO: 63 - protein, 8 aa, Artificial Sequence
  4D8F2 VH-CDR3
GVRGDMDY

SEQ ID NO: 64 - protein, 11 aa, Artificial Sequence
  50H8G3 VH-CDR3
GSYSNYTWFAY

SEQ ID NO: 65 - protein, 11 aa, Artificial Sequence
  5A7B9 VH-CDR3
GENNYYGGSYD

SEQ ID NO: 66 - protein, 14 aa, Artificial Sequence
  7F10B2 VH-CDR3
NVDYSNYLFFPMDY

SEQ ID NO: 67 - protein, 11 aa, Artificial Sequence
  25C5D6F3 VH-CDR3
GSYTNYTWFAY

SEQ ID NO: 68 - protein, 13 aa, Artificial Sequence
  17F3E8 VH-CDR3
VSYSHYVAGALDN

SEQ ID NO: 69 - protein, 17 aa, Artificial Sequence
  82B10C1 VH-CDR3
WGPYYYSTYMRAYLMDA

SEQ ID NO: 70 - protein, 12 aa, Artificial Sequence
  41E4F2 VH-CDR3
VIYYSDYGAMDY

SEQ ID NO: 71 - protein, 12 aa, Artificial Sequence
  41F7H8 VH-CDR3
CVYGNYVGAMDY

SEQ ID NO: 72 - protein, 15 aa, Artificial Sequence
  41H8A8 VH-CDR3
EGITLVVATYWCFDV

SEQ ID NO: 73 - protein, 12 aa, Artificial Sequence
  42A2A6 VH-CDR3
WGYSDFLYYFDY

SEQ ID NO: 74 - protein, 16 aa, Artificial Sequence
  42C3A4 VH-CDR3
GAPLFGGYYKGVYFDY

SEQ ID NO: 75 - protein, 10 aa, Artificial Sequence
  43B2A12 VH-CDR3
SGLLRWFAYW

SEQ ID NO: 76 - protein, 11 aa, Artificial Sequence
  43C4A11 VH-CDR3
RWGYRYYAMDY

SEQ ID NO: 77 - protein, 15 aa, Artificial Sequence
  43D3A9 VH-CDR3
EGVTMVVATYWCFDV

SEQ ID NO: 78 - protein, 9 aa, Artificial Sequence
  43G5G3 VH-CDR3
ANDGYWFAY

SEQ ID NO: 79 - protein, 12 aa, Artificial Sequence
  45C5A1 VH-CDR3
HGGLRVYYAIDY

SEQ ID NO: 80 - protein, 13 aa, Artificial Sequence
  45C9F9 VH-CDR3
FGALMGYYVGFAY

SEQ ID NO: 81 - protein, 8 aa, Artificial Sequence
  45C9F9-2 VH-CDR3
DQLDGFDY

SEQ ID NO: 82 - protein, 8 aa, Artificial Sequence
  49C3B8 VH-CDR3
GLSHAMDY

SEQ ID NO: 83 - protein, 16 aa, Artificial Sequence
  49E4C2 VH-CDR3
GAPLFDGYYKGVYFDY

SEQ ID NO: 84 - protein, 15 aa, Artificial Sequence
  49E4F1 VH-CDR3
EGITLVVTSYWCFDV

SEQ ID NO: 85 - protein, 15 aa, Artificial Sequence
  4D11H2 VH-CDR3
EGITMVVATYWCFDV

SEQ ID NO: 86 - protein, 11 aa, Artificial Sequence
  50B9C10-2 VH-CDR3
GSYRNYTWFAY

SEQ ID NO: 87 - protein, 12 aa, Artificial Sequence
  50C9C2 VH-CDR3
HGGLRVYYAMDY

SEQ ID NO: 88 - protein, 12 aa, Artificial Sequence
  50F11D12 VH-CDR3
EGLRTYYYAMDY

SEQ ID NO: 89 - protein, 8 aa, Artificial Sequence
  54E4A12 VH-CDR3
GGGETGDY

SEQ ID NO: 90 - protein, 10 aa, Artificial Sequence
  76B2E6 VH-CDR3
RTTAGIRFAY

SEQ ID NO: 91 - protein, 12 aa, Artificial Sequence
  53E8B2 VH-CDR3
HGRVFGFGYFDY

SEQ ID NO: 92 - protein, 11 aa, Artificial Sequence
  43F1C10-2 VL-CDR1
KASQSVSNDVA

SEQ ID NO: 93 - protein, 11 aa, Artificial Sequence
  49E9G10 VL-CDR1
LASQTIGTWLA

SEQ ID NO: 94 - protein, 15 aa, Artificial Sequence
  82B10C1 VL-CDR1
RTSQNVDNYGITYMY

SEQ ID NO: 95 - protein, 11 aa, Artificial Sequence
  41E4F2 VL-CDR1
RASENIYSYLA

SEQ ID NO: 96 - protein, 11 aa, Artificial Sequence
  41F7H8 VL-CDR1
KAGQSVSNDVA

SEQ ID NO: 97 - protein, 11 aa, Artificial Sequence
  41H8A8VL-CDR1
KASQNVGTNVA

SEQ ID NO: 98 - protein, 10 aa, Artificial Sequence
  42A2A6 VL-CDR1
RASSSVSYIH

SEQ ID NO: 99 - protein, 11 aa, Artificial Sequence
  42C3A4 VL-CDR1
QASQSVSNEVA

SEQ ID NO: 100 - protein, 10 aa, Artificial Sequence
  43B2A12 VL-CDR1
RASSSVNYMY

SEQ ID NO: 101 - protein, 11 aa, Artificial Sequence
  43C4A11 VL-CDR1
KASQNVGTAVA

SEQ ID NO: 102 - protein, 11 aa, Artificial Sequence
  43G5G3 VL-CDR1
LASQTVGTWLA

SEQ ID NO: 103 - protein, 11 aa, Artificial Sequence
  45C5A1 VL-CDR1
LASQTIGPWLA

SEQ ID NO: 104 - protein, 11 aa, Artificial Sequence
  45C9F9 VL-CDR1
KASEDIYNRLA

SEQ ID NO: 105 - protein, 16 aa, Artificial Sequence
  49C3B8 VL-CDR1
RSSQSIVHTNGNTYLE

SEQ ID NO: 106 - protein, 11 aa, Artificial Sequence
  49E4C2 VL-CDR1
KASQSVSNEVA

SEQ ID NO: 107 - protein, 11 aa, Artificial Sequence
  54E4A12 VL-CDR1
RASQNIYKYLN

SEQ ID NO: 108 - protein, 16 aa, Artificial Sequence
  76B2E6 VL-CDR1
RSSQSFVHSDGNTYLN

SEQ ID NO: 109 - protein, 11 aa, Artificial Sequence
  27H11B5 VL-CDR1
KASQSVTNDVA

SEQ ID NO: 110 - protein, 11 aa, Artificial Sequence
  53E8B2 VL-CDR1
QASQDIGDWVA

SEQ ID NO: 111 - protein, 11 aa, Artificial Sequence
  60C4B9 VL-CDR1
QASQDIGDWLA

SEQ ID NO: 112 - protein, 15 aa, Artificial Sequence
  15B4C6 VL-CDR1
RASKSVSSSGYNFIY

SEQ ID NO: 113 - protein, 11 aa, Artificial Sequence
  33A3E2 VL-CDR1
LTSQTIGTWLA

SEQ ID NO: 114 - protein, 7 aa, Artificial Sequence
  43F1C10-2 VL-CDR2
FASNRYT

SEQ ID NO: 115 - protein, 7 aa, Artificial Sequence
  49E9G10 VL-CDR2
AATSLAD

SEQ ID NO: 116 - protein, 7 aa, Artificial Sequence
  4D8F2 VL-CDR2
YASNRYT

SEQ ID NO: 117 - protein, 7 aa, Artificial Sequence
  7F10B2 VL-CDR2
YASNRFT

SEQ ID NO: 118 - protein, 7 aa, Artificial Sequence
  82B10C1 VL-CDR2
EGSNLAS

SEQ ID NO: 119 - protein, 7 aa, Artificial Sequence
  41E4F2 VL-CDR2
NAKTLAE

SEQ ID NO: 120 - protein, 7 aa, Artificial Sequence
  41H8A8 VL-CDR2
SASYRYS

SEQ ID NO: 121 - protein, 7 aa, Artificial Sequence
  42A2A6 VL-CDR2
ATSNLAS

SEQ ID NO: 122 - protein, 7 aa, Artificial Sequence
  42C3A4 VL-CDR2
YASSRYT

SEQ ID NO: 123 - protein, 7 aa, Artificial Sequence
  43B2A12 VL-CDR2
YTSHLAP

SEQ ID NO: 124 - protein, 7 aa, Artificial Sequence
  43C4A11 VL-CDR2
SASNRYT

SEQ ID NO: 125 - protein, 7 aa, Artificial Sequence
  45C9F9 VL-CDR2
GATSLET

SEQ ID NO: 126 - protein, 7 aa, Artificial Sequence
  49C3B8 VL-CDR2
KVSNRFS

SEQ ID NO: 127 - protein, 7 aa, Artificial Sequence
  54E4A12 VL-CDR2
YTNSLQT

SEQ ID NO: 128 - protein, 7 aa, Artificial Sequence
  76B2E6 VL-CDR2
NISNRLS

SEQ ID NO: 129 - protein, 7 aa, Artificial Sequence
  53E8B2 VL-CDR2
GADRLAD

SEQ ID NO: 130 - protein, 7 aa, Artificial Sequence
  60C4B9 VL-CDR2
GATSLAD

SEQ ID NO: 131 - protein, 7 aa, Artificial Sequence
  15B4C6 VL-CDR2
LASNLES

SEQ ID NO: 132 - protein, 7 aa, Artificial Sequence
  55D3F10 VL-CDR2
GADTLAD

SEQ ID NO: 133 - protein, 9 aa, Artificial Sequence
  43F1C10-2 VL-CDR3
QQDYSSPYT

SEQ ID NO: 134 - protein, 9 aa, Artificial Sequence
  49E9G10 VL-CDR3
QQLYSIPLT

SEQ ID NO: 135 - protein, 9 aa, Artificial Sequence
  4D8F2 VL-CDR3
QQDYSSPFT

SEQ ID NO: 136 - protein, 9 aa, Artificial Sequence
  5A7B9 VL-CDR3
QQDYSSPLT

SEQ ID NO: 137 - protein, 8 aa, Artificial Sequence
  7F10B2 VL-CDR3
QQDYSSRT

SEQ ID NO: 138 - protein, 9 aa, Artificial Sequence
  82B10C1 VL-CDR3
QQSKDYPWT

SEQ ID NO: 139 - protein, 9 aa, Artificial Sequence
  41E4F2 VL-CDR3
QHHYGTPWT

SEQ ID NO: 140 - protein, 9 aa, Artificial Sequence
  41H8A8 VL-CDR3
QQYNNYPYT

SEQ ID NO: 141 - protein, 9 aa, Artificial Sequence
  42A2A6 VL-CDR3
QQWSNNPWT

SEQ ID NO: 142 - protein, 9 aa, Artificial Sequence
  42C3A4 VL-CDR3
QQDYNSPYT

SEQ ID NO: 143 - protein, 9 aa, Artificial Sequence
  43B2A12 VL-CDR3
QQFTSSPYT

SEQ ID NO: 144 - protein, 9 aa, Artificial Sequence
  43C4A11 VL-CDR3
QQYSSYPYT

SEQ ID NO: 145 - protein, 9 aa, Artificial Sequence
  43D3A9 VL-CDR3
QQYNNFPYT

SEQ ID NO: 146 - protein, 9 aa, Artificial Sequence
  43G5G3 VL-CDR3
QQIFSVPYT

SEQ ID NO: 147 - protein, 10 aa, Artificial Sequence
  45C5A1 VL-CDR3
QQLYSDPLYT

SEQ ID NO: 148 - protein, 10 aa, Artificial Sequence
  45C9F9 VL-CDR3
QQYWSTPPYT

SEQ ID NO: 149 - protein, 9 aa, Artificial Sequence
  46H3H3 VL-CDR3
QQLYSTPLT

SEQ ID NO: 150 - protein, 9 aa, Artificial Sequence
  49C3B8 VL-CDR3
FQGSHVPPT

SEQ ID NO: 151 - protein, 9 aa, Artificial Sequence
  49E4F1 VL-CDR3
QQYNSYPYT

SEQ ID NO: 152 - protein, 9 aa, Artificial Sequence
  4D11H2 VL-CDR3
QQYNSFPYT

SEQ ID NO: 153 - protein, 8 aa, Artificial Sequence
  54E4A12 VL-CDR3
FQYSSWYT

SEQ ID NO: 154 - protein, 9 aa, Artificial Sequence
  76B2E6 VL-CDR3
GQASKIPLT

SEQ ID NO: 155 - protein, 9 aa, Artificial Sequence
  53E8B2 VL-CDR3
LQDYSPPFT

SEQ ID NO: 156 - protein, 9 aa, Artificial Sequence
  60C4B9 VL-CDR3
LQDSDPPYT

SEQ ID NO: 157 - protein, 9 aa, Artificial Sequence
  15B4C6 VL-CDR3
QHSRDLPLT

SEQ ID NO: 158 - protein, 9 aa, Artificial Sequence
  55D3F10 VL-CDR3
LQDSSAPYT

SEQ ID NO: 159 - protein, 9 aa, Artificial Sequence
  98C7H11 VL-CDR3
LQDYDPPYT

SEQ ID NO: 160 - protein, 122 aa, Artificial Sequence
  43F1C10-2 VH
EVQLHQSGPELVKPGASVKMSCKASGYTFTNYYMHWVKQSHGKSIEWIGYIHPNNGDTSYNQKFKGKATLTVDKSSSTAYMELRSLTSEDSAVYYCAVVSYSHYVAGAMDYWGQGTSVTVSS

SEQ ID NO: 161 - protein, 120 aa, Artificial Sequence
  49E9G10 VH
EVKLVESGGGLVQPGGSRKLSCAASGFTFSDYGMVWVRQAPGKGLEWVAYIGSGRNTIYYVDTVKGRFTISRDNPKNTLFLQMSSLRSEDTAMYYCAREDNGYSYAMDYWGQGTSVTVSS

SEQ ID NO: 162 - protein, 122 aa, Artificial Sequence
  50B9C10 VH
EVQLHQSGPELVKPGASVKMSCKASGYTFTNYYIHWVKQSHGKSIEWIGYIYPNNGDTSYNQKFKGKATLTVDKSSSTAYMELRSLTSEDSAVYYCAVVSYSNYVAGAMDYWGQGTSVTVSS

SEQ ID NO: 163 - protein, 122 aa, Artificial Sequence
  46F3H4 VH
EVQLHQSGPELVKPGASVKMSCKASGYTFTNYYIHWVKQSHGKSIEWIGYIYPNNGDTTYNQKFKGKATLTVDKSSSIAYMELRSLTSEDSAVYYCAVVSYSNYVAGAMDYWGQGTSVTVSS

SEQ ID NO: 164 - protein, 117 aa, Artificial Sequence
  4D8F2 VH
DVQLQESGPGLVKPSQSLSLTCTVTGYSITSDYAWNWIRQFPGNKLEWMGYISYSGSTSYNPSLKSRISITRDTSKNQFFLQLNSVTTEDTATYYCARGVRGDMDYWGQGTSVTVSS

SEQ ID NO: 165 - protein, 122 aa, Artificial Sequence
  50H8G3 VH
EVNLVESGGGLVQPGGSLRLSCATSGFTFSDFYMEWVRQPPGKRLEWIAASRNKAKDYTTEYSASVKGRFIVSRDTSQSILYLQMNALRAEDTAIYYCARGSYSNYTWFAYWGQGTLVTVSA

SEQ ID NO: 166 - protein, 120 aa, Artificial Sequence
  5A7B9 VH
QIQLVQSGPELKKPGETVKISCKASGYTFTNYGVNWVKQAPGKGLKWMGWINSYSGVPTYADDFKGRFAFSLETSASTAYLQINNLKNEDTATYFCARGENNYYGGSYDWGQGTTLTVSS

SEQ ID NO: 167 - protein, 122 aa, Artificial Sequence
  5F7D7 VH
EVQLHQSGPELVKPGASVKMSCKASGYTFTNYYMHWVKQSHGKSIEWIGYIYPNNGDTSYNQKFKGKATLTVDKSSSTAYMELRSLTSEDSAVYYCAVVSYSNYVAGAMDYWGQGTSVTVSS

SEQ ID NO: 168 - protein, 123 aa, Artificial Sequence
  7F10B2 VH
EVQLQQSGPELVKPGSSVKISCKTSGYTFTDYNMDWVKQSHGKSLEWIGTINPKNGETSDNQKFKAKATLTVDKSSSTAYMELRSLTSEDSAVYYCAKNVDYSNYLFFPMDYWGQGTSVTVSS

SEQ ID NO: 169 - protein, 120 aa, Artificial Sequence
  31E2D4 VH
QIQLVQSGPELKKPGETVKISCKASGYTLTNYGMNWVKQAPGKGLKWMGWINSYSGVPTYADDFKGRFAFSLETSASTAYLQINNLKNEDTATYFCARGENNYYGGSYDWGQGTTLTVSS

SEQ ID NO: 170 - protein, 117 aa, Artificial Sequence
  15H10D12 VH
DVQLQESGPGLVKPSQSLSLTCTVTGYSITSDYAWNWIRQFPGNRLEWMGYINYSGSTDYNPSLKSRISITRDTSKNQFFLQLNSVTTEDTATYYCARGVRGDMDYWGQGTSVTVSS

SEQ ID NO: 171 - protein, 122 aa, Artificial Sequence
  25C5D6F3 VH
EVNLVESGGGLVQPGGSLRLSCATSGFTFSDFYMEWVRQPPGKRLEWIAASRNKANDYTTEYSASVKGRFIVSRDTSQSILYLQMNALRAEDTAIYYCTRGSYTNYTWFAYWGQGTLVTVSA

SEQ ID NO: 172 - protein, 122 aa, Artificial Sequence
  17F3E8 VH
EVQLHQSGPELVKPGASVKMSCKASGYTFTNYYIHWVKQSHGKTIEWIGYIHPNNGDTSYNQKFKDKATLTVDKSSSTAYMDLRSLTSEDSAVYYCSVVSYSHYVAGALDNWGQGTSVTVSS

SEQ ID NO: 173 - protein, 126 aa, Artificial Sequence
  82B10C1 VH
QVKLLQSGAALVKPGDSVKMSCKASGYTFTDYIIHWVKQSHGKSLEWIGYINPFSGGTNYNEKFKSKATLTVDKSSSTAYMEFSRLTSDDSAIYYCARWGPYYYSTYMRAYLMDAWGQGASVTVSS

SEQ ID NO: 174 - protein, 122 aa, Artificial Sequence
  50H8E5C5 VH
EVQLHQSGPELVKPGASVKMSCKASGYTFTNYYMHWVKQSHGKSIEWIGYIYPNNGDTNYNQKFKGKATLTVDKSSSTAYMELRSLTSEDSAVYSCAVVSYSNYVAGAMDYWGQGTSVTVSS

SEQ ID NO: 175 - protein, 121 aa, Artificial Sequence
  41E4F2 VH
DVQLQESGPGLVKPSQSLSLTCTVTGYSITSDYAWNWIRQFPGNKLEWMGYISYSGSTSYNPSLKSRISITRDTSKNQFFLQLNSVTTEDTATYYCASVIYYSDYGAMDYWGQGTSVTVSS

SEQ ID NO: 176 - protein, 121 aa, Artificial Sequence
  41F7H8 VH
DVQFQESGPGLVKPSQSLSLTCTVTGYSITSDYAWNWIRQFPGNKLEWMGYISYSGSTSYNPSLKSRISITRDTSKNQFFLQLNSVTTEDTATYYCARCVYGNYVGAMDYWGQGTSVTVSS

SEQ ID NO: 177 - protein, 124 aa, Artificial Sequence
  41H8A8 VH
EVQLQQSGTELARPGASVKMSCKASGYTFTSNWMHWVKQRPGQGLEWIGAIYPGNSDTSYNQKFKDKAKLTAVTSASTAYMELSSLTNEDSAVYYCTREGITLVVATYWCFDVWGTGTTVTVSS

SEQ ID NO: 178 - protein, 121 aa, Artificial Sequence
  42A2A6 VH
DVQLQESGPGLVKPSQSLSLTCTVTGYSITSDYAWNWIRQFPGNKLEWMGFINYSGTTSYNPSLKSRISLTRDTSKNQFFLQLNSVTTEDTATYYCARWGYSDFLYYFDYWGQGTTLTVSS

SEQ ID NO: 179 - protein, 127 aa, Artificial Sequence
  42C3A4 VH
EVKLEESGGGLVQPGGSMKLSCVASGITFSNYWMDWVRQSPEKGLEWVAEIRLKSNNYATHYAESVKGRFTISRDDSKNSVYLQMNNLRAEDTGIYYCTRGAPLFGGYYKGVYFDYWGQGTTLTVSS

SEQ ID NO: 180 - protein, 118 aa, Artificial Sequence
  43B2A12 VH
QVQLQQPGAELVKPGASVKLSCKASGYNFTGYWMHWVKQRPGQGLEWIGEIDPSDSFTYNNQKFKGKATLTVDKSSNTAYMQLSSLTSEDSAVYYCARSGLLRWFAYWGQGTLVTVSA

SEQ ID NO: 181 - protein, 120 aa, Artificial Sequence
  43C4A11 VH
QVQLQQPGAELVKPGASVKLSCKASGYTFTSYWMHWVKQRPGQGLEWIGEIDPSDSYSYYNQKFKGKATLTVDKSSSTAYMQLSSLTSEDSAVYYCARRWGYRYYAMDYWGQGTSVTVSS

SEQ ID NO: 182 - protein, 124 aa, Artificial Sequence
  43D3A9 VH
EVQLQQSGAELARPGASVKMSCKASGYTFTSNWIHWVKQRPGQGLEWIGAIYPGNNDTSYNQKFKDKAKLTAVTSANTAYMELSSLTNEDSAVYYCTREGVTMVVATYWCFDVWGTGTTVTVSS

SEQ ID NO: 183 - protein, 122 aa, Artificial Sequence
  43F1C10 VH
EVNLVESGGGLVQPGGSLRLSCATSGFTFSDFYMEWVRQPPGKRLEWIAASRDKANDYTTEYSASVKGRFIVSRDTSQSILYLQMNALRAEDTAIYYCARGSYSNYTWFAYWGQGTLVTVSA

SEQ ID NO: 184 - protein, 120 aa, Artificial Sequence
  43G5G3 VH
EVNLVESGGGLVQPGGSLRLSCATSGFTFSDFYMEWVRQPPGKRLEWIASSRNKANDYTTEYNASVKGRFIVSRDTSQSILYLQMNALRAEDTAIYYCARANDGYWFAYWGQGTLVTVSA

SEQ ID NO: 185 - protein, 121 aa, Artificial Sequence
  45C5A1 VH
EVKLVESGGGLVQPGGSLKLSCAASGFTFSNYTMSWVRQTPEKRLELVAEISNSDGSAYYPDTVKGRFTISRDNAKKTLYLQMSSLKSEDTAMYYCARHGGLRVYYAIDYWGQGTSVTVSS

SEQ ID NO: 186 - protein, 122 aa, Artificial Sequence
  45C9F9 VH
QIQLVQSGPELKKPGETVKISCKASGYTFTNYGMNWVKQAPGKGLKWMGWISTYSGVPTYADDFKGRFAFSLETSASTAYLQINNLKNEDSATYFCSRFGALMGYYVGFAYWGQGTLVTVSA

SEQ ID NO: 187 - protein, 116 aa, Artificial Sequence
  45C9F9-2 VH
QVQLKESGPGLVAPSQSLSITCTVSGFSLTSYGVHWVRQPPGKGLEWLGVIWAGGSTNYNSALMSRLSISKDNSKSQVFLKMNSLQTDDTAMYYCARDQLDGFDYWGQGTTLTVSS

SEQ ID NO: 188 - protein, 120 aa, Artificial Sequence
  46H3H3 VH
EVKLVESGGGLVQPGGSRKLSCAASGFTFSDYGMVWVRQAPGKGLEWVAYIGSGRSTIYYADTVKGRFTISRDNPKNTLFLQMSSLRSEDTAMYYCAREDNGYSYAMDYWGQGTSVTVSS

SEQ ID NO: 189 - protein, 116 aa, Artificial Sequence
  49C3B8 VH
EVQLQESGPSLVKPSQTLSLTCSVTGDSITSDNWNWIRKFPGNKLEYMGYISYSGSSYYNPSLKSRISITRDTSKNQYYLQLNSVTSEDTATYYCARGLSHAMDYWGQGTSVTVSS

SEQ ID NO: 190 - protein, 127 aa, Artificial Sequence
  49E4C2 VH
EVKLEESGGGLVQPGGSMKLSCVASGITFSNYWMDWVRQSPEKGLEWVAEIRLKSNNYATHYAESVKGRFTISRDDSKSSVYLQMNNLRAEDTGIYYCTRGAPLFDGYYKGVYFDYWGQGTTLTVSS

SEQ ID NO: 191 - protein, 124 aa, Artificial Sequence
  49E4F1 VH
EVQLQQSGTELARPGASVKMSCKASGYTFTSNWMHWVKQRPGQGLEWIGAIYPGNSDTDYNQKFKDKAKLTAVTSASTAYMELSSLTDEDSAVYYCTREGITLVVTSYWCFDVWGTGTTVTVSS

SEQ ID NO: 192 - protein, 120 aa, Artificial Sequence
  49G4D6 VH
EVKLVESGGGLVQPGGSRKLSCAASGFTFSDYGMVWVRQAPGKGLEWVAYIGSGRNTIYYADTVKGRFTISRDNPKNTLFLQMSSLRSEDTAMYYCAREDNGYSYAMDYWGQGTSVTVSS

SEQ ID NO: 193 - protein, 124 aa, Artificial Sequence
  4D11H2 VH
EVQLQQSGTELARPGASVKMSCKASGYTFTSNWMHWVKQRPGQGLEWIGAIYPGNSDTSYNQKFKDKAKLTAVTSASTAYMELSSLTNEDSAVYYCTREGITMVVATYWCFDVWGTGTTVTVSS

SEQ ID NO: 194 - protein, 122 aa, Artificial Sequence
  50B9C10-2 VH
EVNLVESGGGLVQPGGSLRLSCATSGFTFSDFYMEWVRQPPGKRLEWIAASRNKANDYTTEYSASVKGRFIVSRDTSQSILYLQMNALRAEDTAIYYCARGSYRNYTWFAYWGQGTLVTVSA

SEQ ID NO: 195 - protein, 121 aa, Artificial Sequence
  50C9C2 VH
EVKLVESGGGLVQPGGSLKLSCAASGFTFSNYMMSWVRQTPEKRLELVAEISNSDGSTYYPDTVKGRFTISRDNAKNTLYLQMSSLKSEDTAMYYCARHGGLRVYYAMDYWGQGTSVTVSS

SEQ ID NO: 196 - protein, 121 aa, Artificial Sequence
  50F11D12 VH
EVKLVESGGGSVQPGGSRKLSCAASGFTFSDYGMVWVRQAPGKGLEWIGYISGGSKTVYYADTVKGRFTMSRDNPQNTLFLQMSSLRSEDTAMYYCAREGLRTYYYAMDYWGQGTSVTVSS

SEQ ID NO: 197 - protein, 116 aa, Artificial Sequence
  54E4A12 VH
QVQLKESGPGLVQPSQTLSLTCTVAGFSLTTYNVHWVRQPPGKGLEWMGVIWNTGGTRYNSTLKSRLSISKDTSKSQVFLKMNSLQTEDTATYYCARGGGETGDYWGQGVMVTVSS

SEQ ID NO: 198 - protein, 122 aa, Artificial Sequence
  5F7D7-2 VH
EVNLVESGGGLVQPGGSLRLSCATSGFTFSDFYMEWVRQPPGKRLEWIAASRNKANDYTTEYSASVKGRFIVSRDTSQSILYLQMNALRAEDTAIYYCARGSYSNYTWFAYWGQGTLVTVSA

SEQ ID NO: 199 - protein, 119 aa, Artificial Sequence
  76B2E6 VH
QVHLQQSGAELATPGTSVKLSCKASGYSFTRFYIYWVKQRPGQGLEWIGYIYPGNGDTDYSEKFKGKATLTTDTSSSTAYLLLGSLTPEDSAYYFCATRTTAGIRFAYWGQGTLVTVSS

SEQ ID NO: 200 - protein, 120 aa, Artificial Sequence
  41A6A6 VH
EVKLVESGGGLVQPGGSRKLSCAASGFTFSDYGMVWVRQAPGKGLEWVAYIGSGRSTIYYADTVKGRFTISRDNPKNTLFLQMSSLGSEDTAMYYCAREDNGYSYAMDYWGQGTSVTVSS

SEQ ID NO: 201 - protein, 120 aa, Artificial Sequence
  39C1C4 VH
EVKLVESGGGLVQPGGSRKLSCAASGFTFSDYGMVWVRQAPGKGLEWVAYIGSGRNTIYYADTVKGRFTISRDNPKNTLFLQMSSLRSEDTAMYYCVREDNGYSYAMDYWGQGTSVTVSS

SEQ ID NO: 202 - protein, 120 aa, Artificial Sequence
  49B8G4 VH
EVKLVESGGGLVQPGGSRKLSCAASGFTFSDYGMVWVRQAPGKGLEWIAYIGSGRNTIYYADTVKGRFTISRDNPKNTLFLQMSSLRSEDTAMYYCAREDNGYSYAMDYWGQGTSVTVSS

SEQ ID NO: 203 - protein, 121 aa, Artificial Sequence
  27H11B5 VH
DVQLQESGPGLVKPSQSLSLTCTVTGYSITSDYAWNWIRQFPGNKLEWMGYISYSGSTSYNPSLKSRISITRDTSKNQFFLQLNSVTTEDTATYYCARCVYGNYVGAMDYWGQGTSVTVSS

SEQ ID NO: 204 - protein, 121 aa, Artificial Sequence
  53E8B2 VH
EVQLVESGGGLVQPGRSLKFSCAASGFTFSDYAMAWVRQAPKKGLEWVATIIYDGSSTYYRDSVKGRFTISRDNAKSTLYLQMDSLRSEDTATYYCSRHGRVFGFGYFDYWGQGVMVTVSS

SEQ ID NO: 205 - protein, 121 aa, Artificial Sequence
  60C4B9 VH
EVQLVESGGGLVQPGRSLKFSCAASGFTFSDYAMAWVRQAPKKGLEWVATIIHNGGTTFYRDSVKGRFTISRDNAKTTLYLQMDILRSEDTATYYCVRHGRVFGFGYFDYWGQGVMVTVSS

SEQ ID NO: 206 - protein, 121 aa, Artificial Sequence
  73H11G10 VH
EVQLVESGGGLVQPGRSLKFSCAASGFTFSDYAMAWVRQAPKKGLEWVATIIYDGSSTYYRDSVKGRFTISRDNAKSTLYLQMESLRPEDTATYHCSRHGRIFGFGYFDYWGQGVMVTVSS

SEQ ID NO: 207 - protein, 120 aa, Artificial Sequence
  15B4C6 VH
QVQLKESGPGLVAPSQSLSITCTVSGFSLNGYGVNWVRQPPGKGLEWLGLIWNNGSTDYNSALKSRLTISKDNSKSLVFLKMNSLQTDDTARYYCARDPLYLLRGAMDYWGQGTSVTVSS

SEQ ID NO: 208 - protein, 121 aa, Artificial Sequence
  55D3F10 VH
EVQLVESGGGLVQPGRSLKFSCVVSGFTFSDYAMAWVRQAPKKGLEWVASIIYDGSSTDYRDSVKGRFTISRDNAKSTLYLQMDGLRSEDTATYYCARHGRVFGFGYFDYWGQGVMVTVSS

SEQ ID NO: 209 - protein, 121 aa, Artificial Sequence
  98C7H11 VH
EVQLVESGGGLVQPGRSLKFSCAASGFTFSDYAMAWVRQAPKKGLEWVATIIHDGSTTYYRDSVKGRFTISRDNAKTTLYLQMDILRSEDTATYYCVRHGRVFGFGYFDYWGQGVMVTVSS

SEQ ID NO: 210 - protein, 107 aa, Artificial Sequence
  43F1C10-2 VL
SIVMTQTPKFLLVSAGDRVTITCKASQSVSNDVAWYQQKPGQSPKLLIYFASNRYTGVPDRFTGSGYGTDFTFTISTVQAEDLAVYFCQQDYSSPYTFGGGTKLEIK

SEQ ID NO: 211 - protein, 107 aa, Artificial Sequence
  49E9G10 VL
DIQMTQSPASQSASLGESVTITCLASQTIGTWLAWYQQKPGKSPQLLIYAATSLADGVPSRFSGSGSGTKFSFKISSLQAEDFGSYYCQQLYSIPLTFGVGTKLELK

SEQ ID NO: 212 - protein, 107 aa, Artificial Sequence
  50B9C10 VL
SIVMTQTPKFLLVSAGDRVTITCKASQSVSNDVAWYQQKPGQSPKLLMYFASNRYTGVPDRFTGSGYGTDFTFTISTVQAEDLAVYFCQQDYSSPYTFGGGTKLEIK

SEQ ID NO: 213 - protein, 107 aa, Artificial Sequence
  4D8F2 VL
SIVMTQTPKFLLVSAGDRVTITCKASQSVSNDVAWYQQKPGQSPKLLIYYASNRYTGVPDRFTGSGYGTDFTFTISTVQAEDLAVYFCQQDYSSPFTFGSGTKLEIK

SEQ ID NO: 214 - protein, 107 aa, Artificial Sequence
  5A7B9 VL
SIVMTQTPKFLLVSAGDRVTITCKASQSVSNDVAWYQQKPGQSPKLLIYYASNRYTGVPDRFTGSGYGTDFTFTISTVQAEDLAVYFCQQDYSSPLTFGAGTKLELK

SEQ ID NO: 215 - protein, 107 aa, Artificial Sequence
  5F7D7 VL
SIVMTQTPKFLLVSAGDRVTVTCKASQSVSNDVAWYQQKPGQSPKLLIYFASNRYTGVPDRFTGSGYGTDFTFTISTVQAEDLAVYFCQQDYSSPYTFGGGTKLEIK

SEQ ID NO: 216 - protein, 106 aa, Artificial Sequence
  7F10B2 VL
SIVMTQTPKFLLVSAGDRVTITCKASQSVSNDVAWYQQKPGQSPKLLIYYASNRFTGVPDRFTGSGYGTDFTFTITTVQAEDLAVYFCQQDYSSRTFGGGTKLEIK

SEQ ID NO: 217 - protein, 107 aa, Artificial Sequence
  31E2D4 VL
SIVMTQTPKFLLVSAGDRVTITCKASQSVSNDVAWYQQKPGQSPKLLIYYASNRYTGVPDRFTGSGYGTDFTFTISTVQAEDLAVYFCQQDYSSPLTFGAGTKLELR

SEQ ID NO: 218 - protein, 107 aa, Artificial Sequence
  15H10D12 VL
SIVMTQTPKFLLVSAGDRVTITCKASQSVSNDVAWYQQKPGQSPKLLIYYASNRYTGVPDRFTGSGYGTDFTFTISTVQADDLAVYFCQQDYSSPFTFGSGTKLEIK

SEQ ID NO: 219 - protein, 110 aa, Artificial Sequence
  82B10C1 VL
DIVLTQSPALAVSLEQRVTISCRTSQNVDNYGITYMYWYQQKSRQQPKLLIYEGSNLASGIPARFSGSGSGTDFTLTIDPVEADDIATYYCQQSKDYPWTFGGGTKLELK

SEQ ID NO: 220 - protein, 107 aa, Artificial Sequence
  41E4F2 VL
DIQMTQSPASLSASVGETVTITCRASENIYSYLAWYQQKQGKSPQLLVYNAKTLAEGVPSRFSGSGSGTQFSLKINSLQPEDFGSYYCQHHYGTPWTFGGGTKLEIK

SEQ ID NO: 221 - protein, 107 aa, Artificial Sequence
  41F7H8 VL
SIVMTQTPKFLLVSAGDRVTITCKAGQSVSNDVAWYQQKPGQSPKLLIYYASNRYTGVPDRFTGSGYGTDFTFTISTVQAEDLAVYFCQQDYSSPLTFGAGTKLELK

SEQ ID NO: 222 - protein, 107 aa, Artificial Sequence
  41H8A8 VL
DIVTTQSQKFMSTSVGDRVSVTCKASQNVGTNVAWYQQKPGQSPKALIYSASYRYSGVPDRFTGSGSGTDFTLTISNVQSEDLAEYFCQQYNNYPYTFGGGTKLEIK

SEQ ID NO: 223 - protein, 106 aa, Artificial Sequence
  42A2A6 VL
QIVLSQSPAILSASPGEKVTMTCRASSSVSYIHWYQQKPGYSPKPWIYATSNLASGVPARFSGGGSGTSYSLTISRVEAEDAATYYCQQWSNNPWTFGGGTKLEIK

SEQ ID NO: 224 - protein, 107 aa, Artificial Sequence
  42C3A4 VL
SFVMTQTPKSLLASAGDRVTITCQASQSVSNEVAWYQQKPGQSPKLLIYYASSRYTGVPDRFAGSGYGTDFTFTISTVQAEDLAVYFCQQDYNSPYTFGGGTKLEIK

SEQ ID NO: 225 - protein, 106 aa, Artificial Sequence
  43B2A12 VL
ENVLTQSPAIMSASLGEKVTMSCRASSSVNYMYWYQQKSYASPKLWIYYTSHLAPGVPGRFSGSGSGNSYFLTISSMEGEDAATYYCQQFTSSPYTFGGGTKLEIK

SEQ ID NO: 226 - protein, 107 aa, Artificial Sequence
  43C4A11 VL
DIVMTQSQKFMSTSVGDRVSITCKASQNVGTAVAWYQQKPGQSPKSLIYSASNRYTGVPDRLTGSGSGTDFTLTISNMQSEDLADYFCQQYSSYPYTFGGGTKLEIK

SEQ ID NO: 227 - protein, 107 aa, Artificial Sequence
  43D3A9 VL
DIVMTQSQKFMSTSVGDRVSVTCKASQNVGTNVAWYQQKPGQSPKALIYSASYRYSGVPDRFTGSGSGTDFTLTISNVQSEDLAEYFCQQYNNFPYTFGGGTKLEIK

SEQ ID NO: 228 - protein, 107 aa, Artificial Sequence
  43G5G3 VL
DIQMTQSPASQSASLGESVTITCLASQTVGTWLAWYQQKPGKSPQLLIFAATSLADGVPSRFSGSGSGTKFSFKISSLQAEDFVSYYCQQIFSVPYTFGGGTKLEIK

SEQ ID NO: 229 - protein, 108 aa, Artificial Sequence
  45C5A1 VL
DIQMTQSPASQSASLGESVTITCLASQTIGPWLAWYQQKPGKSPQLLIYAATSLADGVPSRFSGSGSGTKFSFKISSLQAEDFVSYYCQQLYSDPLYTFGGGTKLEMK

SEQ ID NO: 230 - protein, 108 aa, Artificial Sequence
  45C9F9 VL
DIQMTQSSSSFSVSLGDRVTITCKASEDIYNRLAWYQQKPGNAPRLLISGATSLETGVPSRFSGSGSGKDYTLSITSLQTEDVATYYCQQYWSTPPYTFGGGTKLEIK

SEQ ID NO: 231 - protein, 107 aa, Artificial Sequence
  46H3H3 VL
DIQMTQSPASQSASLGESVTITCLASQTIGTWLAWYQQKPGKSPQLLIYAATSLADGVPSRFSGSGSGTKFSFKISSLQAEDFVSYYCQQLYSTPLTFGAGTKLELK

SEQ ID NO: 232 - protein, 112 aa, Artificial Sequence
  49C3B8 VL
DVLMTQTPLSLPVSLGDQASISCRSSQSIVHTNGNTYLEWYLQKPGQSPKLLIYKVSNRFSGVPDRFSGSGSGTDFTLKISRVEAEDLGVYYCFQGSHVPPTFGGGTKLEIK

SEQ ID NO: 233 - protein, 107 aa, Artificial Sequence
  49E4C2 VL
STVMTQTPKSLLASAGDRVAITCKASQSVSNEVAWYQQKPGLSPKLLIYYASNRYTGVPDRFTGSGYGTDFTFTISTVQAEDLAVYFCQQDYNSPYTFGGGTKLEIK

SEQ ID NO: 234 - protein, 107 aa, Artificial Sequence
  49E4F1 VL
DIVMTQSQKFMSTSVGDRVSVTCKASQNVGTNVAWYQQKPGQSPKALIYSASYRYSGVPDRFTGSGSGTDFTLTISNVQSEDLAEYFCQQYNSYPYTFGGGTKLEIK

SEQ ID NO: 235 - protein, 107 aa, Artificial Sequence
  49G4D6 VL
DIQMTQSPASQSASLGESVTITCLASQTIGTWLAWYQQKPGKSPQLLIYAATSLADGVPSRFSSSGSGTKFSFKISSLQAEDFVSYYCQQLYSIPLTFGAGTKLELK

SEQ ID NO: 236 - protein, 107 aa, Artificial Sequence
  4D11H2 VL
DIVMTQSQKFMSTSVGDRVSVTCKASQNVGTNVAWYQQKPGQSPKALIYSASYRYSGVPDRFTGSGSGTDFTLTISNVQSEDLAEYFCQQYNSFPYTFGGGTKLEIK

SEQ ID NO: 237 - protein, 107 aa, Artificial Sequence
  50F11D12 VL
DIQMTQSPASQSASLGESVTITCLASQTIGPWLAWYQQKPGKSPQLLIYAATSLADGVPSRFSGSGSGTKFSFKISSLQAEDFVSYYCQQLYSTPLTFGGGTKLEIK

SEQ ID NO: 238 - protein, 112 aa, Artificial Sequence
  76B2E6 VL
DVVMTQTPVSLSVSLGGQVSISCRSSQSFVHSDGNTYLNWFLQKPGQSPQLLIYNISNRLSGVPDRFSGSGSGTDFTLKISRVEHDDLGVFYCGQASKIPLTFGSGTKLEIK

SEQ ID NO: 239 - protein, 107 aa, Artificial Sequence
  41A6A6 VL
DIQMTQSPASQSASLGESVTITCLASQTIGTWLAWYQQKPGKSPQLLIYAATSLADGVPSRFSGSGSGTKFSFKISSLQAEDFVSYYCQQLYSIPLTFGAGTKLELK

SEQ ID NO: 240 - protein, 107 aa, Artificial Sequence
  49B8G4 VL
DIQMTQSPASQSASLGESVTVTCLASQTIGTWLAWYQQKPGKSPQLLIYAATSLADGVPSRFSGSGSGTKFSFKISSLQAEDFGSYYCQQLYSIPLTFGVGTKLELK

SEQ ID NO: 241 - protein, 107 aa, Artificial Sequence
  27H11B5 VL
SIVMTQTPKFLLVSAGDRVTITCKASQSVTNDVAWYQQKPGQSPKLLIYYASNRYTGVPDRFTGSGYGTDFTFTISTVQTEDLAVYFCQQDYSSPLTFGAGTKLELK

SEQ ID NO: 242 - protein, 107 aa, Artificial Sequence
  53E8B2 VL
DIQMTQSPASLSASLEEIVTITCQASQDIGDWVAWFQQRPGKSPQLLIFGADRLADGVPSRFSGSRSGTQFLLKINRLQVEDPGIYYCLQDYSPPFTFGPGTQLELK

SEQ ID NO: 243 - protein, 107 aa, Artificial Sequence
  60C4B9 VL
DIQMTQSPASLSASLEEIVTITCQASQDIGDWLAWFHQKPGKSPHLLIYGATSLADGVPSRFSGSGSGTQFSLRISGLQVEDPGIYYCLQDSDPPYTFGAGTKLELK

SEQ ID NO: 244 - protein, 111 aa, Artificial Sequence
  15B4C6 VL
DIVLTQSPASLTVSLGQRATISCRASKSVSSSGYNFIYWYQQKPGQPPKLLIYLASNLESGVPARFSGSGSGTDFTLNIHPVEEEDAATYYCQHSRDLPLTFGAGTKLELK

SEQ ID NO: 245 - protein, 107 aa, Artificial Sequence
  55D3F10 VL
NIQMTQSPASLSASLEEVVTITCQASQDIGDWLAWFQQKPGKSPQLLIYGADTLADGVPSRFSGSRSGTQFSLKISRLQVEDPGIYYCLQDSSAPYTFGAGTKLELK

SEQ ID NO: 246 - protein, 107 aa, Artificial Sequence
  98C7H11 VL
DIQMTQSPASLSASLEEIVTITCQASQDIGDWLAWFHQKPGKSPHLLIYGATSLADGVPSRFSGSGSGTQFSLKISGLQVEDPGIYYCLQDYDPPYTFGAGTELELK

SEQ ID NO: 247 - protein, 107 aa, Artificial Sequence
  33A3E2 VL
DIQMTQSPASQSASLGESVTITCLTSQTIGTWLAWYQQKPGKSPQLLIYAATSLADGVPSRFSGSGSGTKFSFKISSLQAEDFVSYYCQQLYSIPLTFGAGTKLELK

SEQ ID NO: 248 - protein, 106 aa, Artificial Sequence
  54E4A12 VL
DIQMTQSPSLLSASVGDRVTLSCRASQNIYKYLNWYQQKLGEAPKLLIHYTNSLQTGIPSRFSGSGSGTDFTLTISSLQPEDVAIYFCFQYSSWYTFGAGTKLELK

SEQ ID NO: 249 - protein, 5 aa, Artificial Sequence
  85E10B9 VH-CDR1
NHYLH

SEQ ID NO: 250 - protein, 20 aa, Artificial Sequence
  85E10B9 VH-CDR2
WIGWIGPTNGDTSYAQKFKD

SEQ ID NO: 251 - protein, 10 aa, Artificial Sequence
  85E10B9 VH-CDR3
EGYFTNYFDY

SEQ ID NO: 252 - protein, 11 aa, Artificial Sequence
  85E10B9 VL-CDR1
KASQNVIKYLA

SEQ ID NO: 253 - protein, 7 aa, Artificial Sequence
  85E10B9 VL-CDR2
NASSLQT

SEQ ID NO: 254 - protein, 8 aa, Artificial Sequence
  85E10B9 VL-CDR3
LQYNSWWT

SEQ ID NO: 255 - protein, 119 aa, Artificial Sequence
  85E10B9 VH
QVQLQQSGAELVKPAASVTLSCKASGYTFTNHYLHWVKQSPSQGLEWIGWIGPTNGDTSYAQKFKDKATMTVDKSASTTYLQLSSLTSEDSAVYYCAREGYFTNYFDYWGQGVMVTVSS

SEQ ID NO: 256 - protein, 106 aa, Artificial Sequence
  85E10B9 VL
DIHMTQSPSFLSASVGDRVTLSCKASQNVIKYLAWYQQKLGEAPKLLIYNASSLQTGIPSRFSGSGSGADFTLTISSLQPEDVATYFCLQYNSWWTFGGGTKLELK

SEQ ID NO: 257 - protein, 327 aa, Artificial Sequence
  Human IgG4 heavy chain constant region
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 258 - protein, 107 aa, Artificial Sequence [sequence omitted]

SEQ ID NO: 259 - protein, 330 aa, Artificial Sequence [sequence omitted]

SEQ ID NO: 260 - protein, 120 aa, Artificial Sequence
  46H3H3-VH1
EVQLVESGGGLVQPGGSLRLSCAASGFTFSDYGMVWVRQAPGKGLEWVSYIGSGRSTIYYADTVKGRFTISRDNAKNSLYLQMNSLRAEDTAVYYCAREDNGYSYAMDYWGQGTLVTVSS

SEQ ID NO: 261 - protein, 120 aa, Artificial Sequence
  46H3H3-VH2
QVQLVESGGGLVKPGGSLRLSCAASGFTFSDYGMVWIRQAPGKGLEWVSYIGSGRSTIYYADTVKGRFTISRDNAKNSLYLQMNSLRAEDTAVYYCAREDNGYSYAMDYWGQGTLVTVSS

SEQ ID NO: 262 - protein, 120 aa, Artificial Sequence
  46H3H3-VH3
EVQLVESGGGLVKPGGSLRLSCAASGFTFSDYGMVWVRQAPGKGLEWVSYIGSGRSTIYYADTVKGRFTISRDNAKNSLYLQMNSLRAEDTAVYYCAREDNGYSYAMDYWGQGTLVTVSS

SEQ ID NO: 263 - protein, 127 aa, Artificial Sequence
  42C3A4-VH1
EVQLVESGGGLVQPGGSLKVSCAASGITFSNYWMDWVRQASGKGLEWVGEIRLKSNNYATHYAESVKGRFTISRDDSKNTAYLQMNSLKTEDTAVYYCTRGAPLFGGYYKGVYFDYWGQGTLVTVSS

SEQ ID NO: 264 - protein, 127 aa, Artificial Sequence
  42C3A4-VH2
EVQLVESGGGLVQPGGSLRLSCAASGITFSNYWMDWVRQAPGKGLEWVAEIRLKSNNYATHYAESVKGRFTISRDNAKNSLYLQMNSLRAEDTAVYYCARGAPLFGGYYKGVYFDYWGQGTLVTVSS

SEQ ID NO: 265 - protein, 127 aa, Artificial Sequence
  42C3A4-VH3
EVQLVESGGGLVKPGGSLRLSCAASGITFSNYWMDWVRQAPGKGLEWVGEIRLKSNNYATHYAESVKGRFTISRDDSKNTLYLQMNSLKTEDTAVYYCTTGAPLFGGYYKGVYFDYWGQGTLVTVSS

SEQ ID NO: 266 - protein, 117 aa, Artificial Sequence
  4D8F2-VH1
QVQLQESGPGLVKPSGTLSLTCAVSGYSITSDYAWNWVRQPPGKGLEWIGYISYSGSTSYNPSLKSRVTISVDKSKNQFSLKLSSVTAADTAVYYCARGVRGDMDYWGQGTLVTVSS

SEQ ID NO: 267 - protein, 117 aa, Artificial Sequence
  4D8F2-VH1.1
QVQLQESGPGLVKPSGTLSLTCAVSGYSITSDYAWNWVRQPPGKGLEWIGYISYSGSTSYNPSLKSRVTISVDKSKNQFSLKLSSVTAADTAVYYCARGVRADMDYWGQGTLVTVSS

SEQ ID NO: 268 - protein, 117 aa, Artificial Sequence
  4D8F2-VH1.2
QVQLQESGPGLVKPSGTLSLTCAVSGYSITSDYAWNWVRQPPGKGLEWIGYISYSGSTSYNPSLKSRVTISVDKSKNQFSLKLSSVTAADTAVYYCARGVRSDMDYWGQGTLVTVSS

SEQ ID NO: 269 - protein, 117 aa, Artificial Sequence
  4D8F2-VH1.3
QVQLQESGPGLVKPSGTLSLTCAVSGYSITSDYAWNWVRQPPGKGLEWIGYISYSGSTSYNPSLKSRVTISVDKSKNQFSLKLSSVTAADTAVYYCARGVRGEMDYWGQGTLVTVSS

SEQ ID NO: 270 - protein, 117 aa, Artificial Sequence
  4D8F2-VH2
QVQLQESGPGLVKPSQTLSLTCTVSGYSITSDYAWNWIRQPPGKGLEWIGYISYSGSTSYNPSLKSRVTISVDTSKNQFSLKLSSVTAADTAVYYCARGVRGDMDYWGQGTLVTVSS

SEQ ID NO: 271 - protein, 117 aa, Artificial Sequence
  4D8F2-VH3
QVQLQQWGAGLLKPSETLSLTCAVYGYSITSDYAWNWIRQPPGKGLEWIGYISYSGSTSYNPSLKSRVTISVDTSKNQFSLKLSSVTAADTAVYYCARGVRGDMDYWGQGTLVTVSS

SEQ ID NO: 272 - protein, 121 aa, Artificial Sequence
  42A2A6-VH1
QVQLQESGPGLVKPSQTLSLTCTVSGYSITSDYAWNWIRQPPGKGLEWIGFINYSGTTSYNPSLKSRVTISVDTSKNQFSLKLSSVTAADTAVYYCARWGYSDFLYYFDYWGQGTLVTVSS

SEQ ID NO: 273 - protein, 121 aa, Artificial Sequence
  42A2A6-VH2
QVQLQESGPGLVKPSETLSLTCTVSGYSITSDYAWNWIRQPPGKGLEWIGFINYSGTTSYNPSLKSRVTISVDTSKNQFSLKLSSVTAADTAVYYCARWGYSDFLYYFDYWGQGTLVTVSS

SEQ ID NO: 274 - protein, 120 aa, Artificial Sequence
  5A7B9-VH1
EVQLVESGSELKKPGASVKVSCKASGYTFTNYGVNWVRQAPGQGLEWMGWINSYSGVPTYADDFKGRFVFSLDTSVSTAYLQISSLKAEDTAVYYCARGENNYYGGSYDWGQGTTVTVSS

SEQ ID NO: 275 - protein, 120 aa, Artificial Sequence
  5A7B9-VH2
QVQVVQSGAEVKQPGASVKVSCKASGYTFTNYGVNWVRQAPGQGLEWMGWINSYSGVPTYADDFKGRVTMTRDTSTSTAYMELRSLRSDDTAVYYCARGENNYYGGSYDWGQGTLVTVSS

SEQ ID NO: 276 - protein, 120 aa, Artificial Sequence
  5A7B9-VH3
QVQLVQSGAEVKKPGASVKVSCKASGYTFTNYGVNWVRQAPGQRLEWMGWINSYSGVPTYADDFKGRVTITRDTSASTAYMELSSLRSEDTAVYYCARGENNYYGGSYDWGQGTLVTVSS

SEQ ID NO: 277 - protein, 120 aa, Artificial Sequence
  5A7B9-VH4
QVQLVQSGAEVKKPGASVKVSCKASGYTFTNYGVNWVRQATGQGLEWMGWINSYSGVPTYADDFKGRVTMTRNTSISTAYMELSSLRSEDTAVYYCARGENNYYGGSYDWGQGTTVTVSS

SEQ ID NO: 278 - protein, 120 aa, Artificial Sequence
  5A7B9-VH3.1
QVQLVQSGAEVKKPGASVKVSCKASGYTFTNYGVNWVRQAPGQRLEWMGWINSYSGVPTYADDFKGRVTITRDTSASTAYLELSSLRSEDTAVYYCARGENNYYGGSYDWGQGTLVTVSS

SEQ ID NO: 279 - protein, 120 aa, Artificial Sequence
  5A7B9-VH3.2
QVQLVQSGAEVKKPGASVKVSCKASGYTFTNYGVNWVRQAPGQRLEWMGWINSYSGVPTYADDFKGRFTITRDTSASTAYMELSSLRSEDTAVYYCARGENNYYGGSYDWGQGTLVTVSS

SEQ ID NO: 280 - protein, 120 aa, Artificial Sequence
  5A7B9-VH3.3
QVQLVQSGAEVKKPGASVKVSCKASGYTFTNYGVNWVRQAPGQRLEWMGWINSYSGVPTYADDFKGRFTITRDTSASTAYLELSSLRSEDTAVYYCARGENNYYGGSYDWGQGTLVTVSS

SEQ ID NO: 281 - protein, 120 aa, Artificial Sequence
  5A7B9-VH3.6
QVQLVQSGAEVKKPGASVKVSCKASGYTFTNYGVNWVRQAPGQRLEWMGWINSYSGVPTYADDFKGRFTFTRDTSASTAYMELSSLRSEDTAVYYCARGENNYYGGSYDWGQGTLVTVSS

SEQ ID NO: 282 - protein, 120 aa, Artificial Sequence
  5A7B9-VH3.7
QVQLVQSGAEVKKPGASVKVSCKASGYTFTNYGVNWVRQAPGQRLEWMGWINSYSGVPTYADDFKGRFTFTLDTSASTAYMELSSLRSEDTAVYYCARGENNYYGGSYDWGQGTLVTVSS

SEQ ID NO: 283 - protein, 122 aa, Artificial Sequence
  17F3E8-VH1.4
EVQLVESGAEVKKPGASVKVSCKASGYTFTNYYIHWVRQAPGQGLEWIGYIHPNNGDTSYNQKFKDRATLTVDTSTSTVYMELSSLRSEDTAVYYCAVVSYSHYVAGALDNWGQGTLVTVSS

SEQ ID NO: 284 - protein, 122 aa, Artificial Sequence
  17F3E8-VH1.5
EVQLVESGAEVKKPGASVKVSCKASGYTFTNYYIHWVRQAPGQGIEWIGYIHPNNGDTSYNQKFKDRATLTVDTSTSTVYMELSSLRSEDTAVYYCAVVSYSHYVAGALDNWGQGTLVTVSS

SEQ ID NO: 285 - protein, 122 aa, Artificial Sequence
  5F7D7-VH3
QVQLVQSGAEVKKPGASVKVSCKASGYTFTNYYMHWVRQAPGQRLEWIGYIYPNNGDTSYNQKFKGRVTITVDTSASTAYMELSSLRSEDTAVYYCAVVSYSNYVAGAMDYWGQGTLVTVSS

SEQ ID NO: 286 - protein, 122 aa, Artificial Sequence
  5F7D7-VH5
QVQLVQSGAEVKKPGASVKVSCKASGYTFTNYYMHWVRQAHGQSLEWIGYIYPNNGDTSYNQKFKGRVTITVDTSASTAYMELSSLRSEDTAVYYCAVVSYSNYVAGAMDYWGQGTLVTVSS

SEQ ID NO: 287 - protein, 122 aa, Artificial Sequence
  5F7D7-VH6
QVQLVQSGAEVKKPGASVKVSCKASGYTFTNYYMHWVRQAHGKSLEWIGYIYPNNGDTSYNQKFKGRATITVDTSASTAYMELSSLRSEDTAVYYCAVVSYSNYVAGAMDYWGQGTLVTVSS

SEQ ID NO: 288 - protein, 120 aa, Artificial Sequence
  31E2D4-VH1
EVQLVESGSELKKPGASVKVSCKASGYTLTNYGMNWVRQAPGQGLEWMGWINSYSGVPTYADDFKGRFVFSLDTSVSTAYLQISSLKAEDTAVYYCARGENNYYGGSYDWGQGTTVTVSS

SEQ ID NO: 289 - protein, 120 aa, Artificial Sequence
  31E2D4-VH2
QVQVVQSGAEVKQPGASVKVSCKASGYTLTNYGMNWVRQAPGQGLEWMGWINSYSGVPTYADDFKGRVTMTRDTSTSTAYMELRSLRSDDTAVYYCARGENNYYGGSYDWGQGTLVTVSS

SEQ ID NO: 290 - protein, 120 aa, Artificial Sequence
  31E2D4-VH3
QVQLVQSGAEVKKPGASVKVSCKASGYTLTNYGMNWVRQAPGQRLEWMGWINSYSGVPTYADDFKGRVTITRDTSASTAYMELSSLRSEDTAVYYCARGENNYYGGSYDWGQGTLVTVSS

SEQ ID NO: 291 - protein, 120 aa, Artificial Sequence
  31E2D4-VH4
QVQLVQSGAEVKKPGASVKVSCKASGYTLTNYGMNWVRQATGQGLEWMGWINSYSGVPTYADDFKGRVTMTRNTSISTAYMELSSLRSEDTAVYYCARGENNYYGGSYDWGQGTTVTVSS

SEQ ID NO: 292 - protein, 123 aa, Artificial Sequence
  7F10B2-VH1
EVQLVESGAEVRKPGASVKVSCKASGYTFTDYNMDWVRQAPGQGLEWMGTINPKNGETSDNQKFKARVTMTRDTSTSTVYMELSSLRSEDTAVYYCARNVDYSNYLFFPMDYWGQGTLVTVSS

SEQ ID NO: 293 - protein, 123 aa, Artificial Sequence
  7F10B2-VH2
QVQLVQSGAEVKKPGASVKVSCKASGYTFTDYNMDWVRQAPGQRLEWMGTINPKNGETSDNQKFKARVTITRDTSASTAYMELSSLRSEDTAVYYCARNVDYSNYLFFPMDYWGQGTLVTVSS

SEQ ID NO: 294 - protein, 123 aa, Artificial Sequence
  7F10B2-VH3
QVQLVQSGAEVKKPGASVKVSCKASGYTFTDYNMDWVRQAPGQGLEWMGTINPKNGETSDNQKFKARVTMTRDTSISTAYMELSRLRSDDTAVYYCARNVDYSNYLFFPMDYWGQGTLVTVSS

SEQ ID NO: 295 - protein, 123 aa, Artificial Sequence
  7F10B2-VH5
QVQLVQSGAEVKKPGASVKVSCKASGYTFTDYNMDWVRQAPGQGLEWMGTINPKNGETSDNQKFKARVTVTTDTSTSTAYMELRSLRSDDTAVYYCARNVDYSNYLFFPMDYWGQGTTVTVSS

SEQ ID NO: 296 - protein, 123 aa, Artificial Sequence
  7F10B2-VH6
QVQLVQSGAEVKKPGSSVKVSCKASGYTFTDYNMDWVRQAPGQGLEWMGTINPKNGETSDNQKFKARVTITADKSTSTAYMELSSLRSEDTAVYYCARNVDYSNYLFFPMDYWGQGTTVTVSS

SEQ ID NO: 297 - protein, 107 aa, Artificial Sequence
  46H3H3-VL1
DIQMTQSPSSLSASVGDRVTITCLASQTIGTWLAWYQQKPGKAPKLLIYAATSLADGVPSRFSGSGSGTDFTLTISSLQPEDFATYYCQQLYSTPLTFGGGTKLEIK

SEQ ID NO: 298 - protein, 107 aa, Artificial Sequence
  46H3H3-VL2
DIQMTQSPSSVSASVGDRVTITCLASQTIGTWLAWYQQKPGKAPKLLIYAATSLADGVPSRFSGSGSGTDFTLTISSLQPEDFATYYCQQLYSTPLTFGGGTKVEIK

SEQ ID NO: 299 - protein, 107 aa, Artificial Sequence
  46H3H3-VL3
DIQMTQSPSTLSASVGDRVTITCLASQTIGTWLAWYQQKPGKAPKLLIYAATSLADGVPSRFSGSGSGTEFTLTISSLQPDDFATYYCQQLYSTPLTFGGGTKVEIK

SEQ ID NO: 300 - protein, 107 aa, Artificial Sequence
  42C3A4-VL1
DIQMTQSPSSLSASVGDRVTITCQASQSVSNEVAWYQQKPGKAPKLLIYYASSRYTGVPSRFSGSGSGTDFTFTISSLQPEDIATYYCQQDYNSPYTFGQGTKLEIK

SEQ ID NO: 301 - protein, 107 aa, Artificial Sequence
  42C3A4-VL2
DIQMTQSPSSLSASVGDRVTITCQASQSVSNEVAWYQQKPGKAPKLLIYYASSRYTGVPSRFSGSGSGTDFTLTISSLQPEDFATYYCQQDYNSPYTFGQGTKLEIK

SEQ ID NO: 302 - protein, 107 aa, Artificial Sequence
  42C3A4-VL3
DIQMTQSPSSLSASVGDRVTITCQASQSVSNEVAWYQQKPGKVPKLLIYYASSRYTGVPSRFSGSGSGTDFTLTISSLQPEDVATYYCQQDYNSPYTFGQGTKLEIK

SEQ ID NO: 303 - protein, 107 aa, Artificial Sequence
  4D8F2-VL1
DIQMTQSPSSLSASVGDRVTITCKASQSVSNDVAWYQQKPGKAPKLLIYYASNRYTGVPSRFSGSGSGTDFTLTISSLQPEDFATYYCQQDYSSPFTFGQGTKLEIK

SEQ ID NO: 304 - protein, 107 aa, Artificial Sequence
  4D8F2-VL2
DIQLTQSPSSLSASVGDRVTITCKASQSVSNDVAWYQQKPGKAPKLLIYYASNRYTGVPSRFSGSGSGTDFTFTISSLQPEDFATYYCQQDYSSPFTFGQGTKVEIK

SEQ ID NO: 305 - protein, 107 aa, Artificial Sequence
  4D8F2-VL3
EIVMTQSPATLSVSPGERATLSCKASQSVSNDVAWYQQKPGQAPRLLIYYASNRYTGIPARFSGSGSGTEFTLTINSLQSEDFAVYYCQQDYSSPFTFGQGTKLEIK

SEQ ID NO: 306 - protein, 106 aa, Artificial Sequence
  42A2A6-VL1
EIVLTQSPATLSLSPGERATLSCRASSSVSYIHWYQQKPGQAPRLLIYATSNLASGIPARFSGSGSGTDFTLTISSLEPEDFAVYYCQQWSNNPWTFGQGTKVEIK

SEQ ID NO: 307 - protein, 106 aa, Artificial Sequence
  42A2A6-VL2
EIVLTQSPGTLSLSPGERATLSCRASSSVSYIHWYQQKPGQAPRLLIYATSNLASGIPDRFSGSGSGTDFTLTISRLEPEDFAVYYCQQWSNNPWTFGGGTKVEIK

SEQ ID NO: 308 - protein, 106 aa, Artificial Sequence
  42A2A6-VL3
DIQMTQSPSSLSASVGDRVTITCRASSSVSYIHWYQQKPGKAPKLLIYATSNLASGVPSRFSGSGSGTDFTLTISSLQPEDFATYYCQQWSNNPWTFGGGTKLEIK

SEQ ID NO: 309 - protein, 106 aa, Artificial Sequence
  42A2A6-VL4
EIVMTQSPATLSVSPGERATLSCRASSSVSYIHWYQQKPGQAPRLLIYATSNLASGIPARFSGSGSGTEFTLTINSLQSEDFAVYYCQQWSNNPWTFGQGTKLEIK

SEQ ID NO: 310 - protein, 107 aa, Artificial Sequence
  5A7B9-VL1
DIQMTQSPSSLSASVGDRVTITCKASQSVSNDVAWYQQKPGKAPKLLIYYASNRYTGVPSRFSGSGSGTDFTLTISSLQPEDFATYYCQQDYSSPLTFGGGTKLEIK

SEQ ID NO: 311 - protein, 107 aa, Artificial Sequence
  5A7B9-VL2
DIQLTQSPSSLSASVGDRVTITCKASQSVSNDVAWYQQKPGKAPKLLIYYASNRYTGVPSRFSGSGSGTDFTFTISSLQPEDFATYYCQQDYSSPLTFGQGTKVEIK

SEQ ID NO: 312 - protein, 107 aa, Artificial Sequence
  5A7B9-VL3
EIVMTQSPATLSVSPGERATLSCKASQSVSNDVAWYQQKPGQAPRLLIYYASNRYTGIPARFSGSGSGTEFTLTINSLQSEDFAVYYCQQDYSSPLTFGQGTKLEIK

SEQ ID NO: 313 - protein, 107 aa, Artificial Sequence
  5A7B9-VL4
EIVLTQSPATLSLSPGERATLSCKASQSVSNDVAWYQQKPGQAPRLLIYYASNRYTGIPARFSGSGSGTDFTLTISSLEPEDFAVYYCQQDYSSPLTFGGGTKVEIK

SEQ ID NO: 314 - protein, 107 aa, Artificial Sequence
  5A7B9-VL1.1
DIQMTQSPSSLSASVGDRVTITCKASQSVSNDVAWYQQKPGKAPKLLIYYASNRYTGVPSRFSGSGSGTDFTLTISSLQPEDFATYFCQQDYSSPLTFGGGTKLEIK

SEQ ID NO: 315 - protein, 107 aa, Artificial Sequence
  5A7B9-VL1.2
DIQMTQSPSSLSASVGDRVTITCKASQSVSNDVAWYQQKPGKAPKLLIYYASNRYTGVPSRFSGSGSGTDFTFTISSLQPEDFATYFCQQDYSSPLTFGGGTKLEIK

SEQ ID NO: 316 - protein, 107 aa, Artificial Sequence
  5A7B9-VL1.3
DIQMTQSPSSLSASVGDRVTITCKASQSVSNDVAWYQQKPGKAPKLLIYYASNRYTGVPSRFSGSGSGTDFTFTISSLQPEDFATYYCQQDYSSPLTFGGGTKLEIK

SEQ ID NO: 317 - protein, 107 aa, Artificial Sequence
  5A7B9-VL2.1
DIQLTQSPSSLSASVGDRVTITCKASQSVSNDVAWYQQKPGKAPKLLIYYASNRYTGVPSRFSGSGSGTDFTFTISSLQPEDFATYFCQQDYSSPLTFGQGTKVEIK

SEQ ID NO: 318 - protein, 107 aa, Artificial Sequence
  5F7D7-VL2.1
DIQLTQSPSSLSASVGDRVTITCKASQSVSNDVAWYQQKPGKAPKLLIYFASNRYTGVPSRFSGSGSGTDFTFTISSLQPEDFATYFCQQDYSSPYTFGQGTKVEIK

SEQ ID NO: 319 - protein, 107 aa, Artificial Sequence
  5F7D7-VL2
DIQLTQSPSSLSASVGDRVTITCKASQSVSNDVAWYQQKPGKAPKLLIYFASNRYTGVPSRFSGSGSGTDFTFTISSLQPEDFATYYCQQDYSSPYTFGQGTKVEIK

SEQ ID NO: 320 - protein, 107 aa, Artificial Sequence
  5F7D7-VL5
DIQMTQSPSSLSASVGDRVTITCKASQSVSNDVAWYQQKPGKAPKLLIYFASNRYTGVPSRFSGSGYGTDFTFTISSLQPEDIATYFCQQDYSSPYTFGGGTKLEIK

SEQ ID NO: 321 - protein, 106 aa, Artificial Sequence
  7F10B2-VL1
DIQMTQSPSSLSASVGDRVTITCKASQSVSNDVAWYQQKPGKAPKLLIYYASNRFTGVPSRFSGSGSGTDFTFTISSLQPEDIATYYCQQDYSSRTFGGGTKVEIK

SEQ ID NO: 322 - protein, 106 aa, Artificial Sequence
  7F10B2-VL2
EIVMTQSPATLSVSPGERATLSCKASQSVSNDVAWYQQKPGQAPRLLIYYASNRFTGIPARFSGSGSGTDFTLTISSLQSEDFAVYYCQQDYSSRTFGQGTKVEIK

SEQ ID NO: 323 - protein, 106 aa, Artificial Sequence
  7F10B2-VL3
DIQMTQSPSSLSASVGDRVTITCKASQSVSNDVAWYQQKPGKAPKLLIYYASNRFTGVPSRFSGSGSGTDFTLTISSLQPEDFATYYCQQDYSSRTFGGGTKVEIK

SEQ ID NO: 324 - protein, 107 aa, Artificial Sequence
  49E4C2 -VL1
DIQMTQSPSSLSASVGDRVTITCKASQSVSNEVAWYQQKPGKAPKLLIYYASNRYTGVPSRFSGSGSGTDFTFTISSLQPEDIATYYCQQDYNSPYTFGGGTKLEIK

SEQ ID NO: 325 - protein, 127 aa, Artificial Sequence
  49E4C2 -VH1
EVQLVESGGGLVQPGGSLKVSCAASGITFSNYWMDWVRQASGKGLEWVGEIRLKSNNYATHYAESVKGRFTISRDDSKNTAYLQMNSLKTEDTAVYYCTRGAPLFDGYYKGVYFDYWGQGTLVTVSS

SEQ ID NO: 326 - protein, 121 aa, Artificial Sequence
  53E8B2-VH1
QVQLVESGGGVVQPGRSLRLSCAASGFTFSDYAMAWVRQAPGKGLEWVATIIYDGSSTYYRDSVKGRFTISRDNSKNTLYLQMNSLRAEDTAVYYCARHGRVFGFGYFDYWGQGTLVTVSS

SEQ ID NO: 327 - protein, 121 aa, Artificial Sequence
  53E8B2-VH2
EVQLVESGGGLVQPGGSLRLSCAASGFTFSDYAMAWVRQAPGKGLEWVSTIIYDGSSTYYRDSVKGRFTISRDNSKNTLYLQMNSLRAEDTAVYYCAKHGRVFGFGYFDYWGQGTLVTVSS

SEQ ID NO: 328 - protein, 121 aa, Artificial Sequence
  53E8B2-VH3
EVQLVESGGGLVQPGGSLRLSCAASGFTFSDYAMAWVRQAPGKGLEWVATIIYDGSSTYYRDSVKGRFTISRDNAKNSLYLQMNSLRAEDTAVYYCARHGRVFGFGYFDYWGQGTLVTVSS

SEQ ID NO: 329 - protein, 119 aa, Artificial Sequence
  85E10B9-VH1
QVQLVQSGAEVKKPGASVKVSCKASGYTFTNHYLHWVRQAPGQGLEWMGWIGPTNGDTSYAQKFKDRVTMTRDTSISTAYMELSRLRSDDTAVYYCAREGYFTNYFDYWGQGTLVTVSS

SEQ ID NO: 330 - protein, 119 aa, Artificial Sequence
  85E10B9-VH3
QVQLVQSGAEVKKPGASVKVSCKASGYTFTNHYLHWVRQAPGQRLEWMGWIGPTNGDTSYAQKFKDRVTITRDTSASTAYMELSSLRSEDTAVYYCAREGYFTNYFDYWGQGTLVTVSS

SEQ ID NO: 331 - protein, 121 aa, Artificial Sequence
  60C4B9-VH1
QVQLVESGGGVVQPGRSLRLSCAASGFTFSDYAMAWVRQAPGKGLEWVATIIHNGGTTFYRDSVKGRFTISRDNSKNTLYLQMNSLRAEDTAVYYCARHGRVFGFGYFDYWGQGTLVTVSS

SEQ ID NO: 332 - protein, 121 aa, Artificial Sequence
  98C7H11-VH3
EVQLVESGGGLVQPGGSLRLSCAASGFTFSDYAMAWVRQAPGKGLEWVATIIHDGSTTYYRDSVKGRFTISRDNAKNSLYLQMNSLRAEDTAVYYCARHGRVFGFGYFDYWGQGTLVTVSS

SEQ ID NO: 333 - protein, 121 aa, Artificial Sequence
  55D3F10-VH1
QVQLVESGGGVVQPGRSLRLSCAASGFTFSDYAMAWVRQAPGKGLEWVASIIYDGSSTDYRDSVKGRFTISRDNSKNTLYLQMNSLRAEDTAVYYCARHGRVFGFGYFDYWGQGTLVTVSS

SEQ ID NO: 334 - protein, 121 aa, Artificial Sequence
  55D3F10-VH2
EVQLVESGGGLVQPGGSLRLSCAASGFTFSDYAMAWVRQAPGKGLEWVSSIIYDGSSTDYRDSVKGRFTISRDNSKNTLYLQMNSLRAEDTAVYYCAKHGRVFGFGYFDYWGQGTLVTVSS

SEQ ID NO: 335 - protein, 121 aa, Artificial Sequence
  73H11G10-VH1
QVQLVESGGGVVQPGRSLRLSCAASGFTFSDYAMAWVRQAPGKGLEWVATIIYDGSSTYYRDSVKGRFTISRDNSKNTLYLQMNSLRAEDTAVYYCARHGRIFGFGYFDYWGQGTLVTVSS

SEQ ID NO: 336 - protein, 107 aa, Artificial Sequence
  53E8B2-VL3
DIQMTQSPSTLSASVGDRVTITCQASQDIGDWVAWYQQKPGKAPKVLIYGADRLADGVPSRFSGSGSGTEFTLTISSLQPDDFATYYCLQDYSPPFTFGPGTKLEIK

SEQ ID NO: 337 - protein, 107 aa, Artificial Sequence
  53E8B2-VL6
DIQMTQSPSSLSASVGDRVTITCQASQDIGDWVAWYQQKPGKAPKRLIYGADRLADGVPSRFSGSGSGTEFTLTISSLQPEDFATYYCLQDYSPPFTFGQGTKVEIK

SEQ ID NO: 338 - protein, 107 aa, Artificial Sequence
  53E8B2-VL2
DIQMTQSPSSVSASVGDRVTITCQASQDIGDWVAWYQQKPGRAPKLLIYGADRLADGVPSRFSGSGSGTDFTLTISSLQPEDFATYYCLQDYSPPFTFGQGTKLEIK

SEQ ID NO: 339 - protein, 106 aa, Artificial Sequence
  85E10B9-VL1
DIQMTQSPSSLSASVGDRVTITCKASQNVIKYLAWYQQKPGKVPKLLIYNASSLQTGVPSRFSGSGSGTDFTLTISSLQPEDVATYYCLQYNSWWTFGGGTKVEIK

SEQ ID NO: 340 - protein, 106 aa, Artificial Sequence
  85E10B9-VL2
DIQLTQSPSFLSASVGDRVTITCKASQNVIKYLAWYQQKPGKAPKLLIYNASSLQTGVPSRFSGSGSGTEFTLTISSLQPEDFATYYCLQYNSWWTFGGGTKVEIK

SEQ ID NO: 341 - protein, 106 aa, Artificial Sequence
  85E10B9-VL6
DIQMTQSPSSLSASVGDRVTITCKASQNVIKYLAWYQQKPGKAPKRLIYNASSLQTGVPSRFSGSGSGTEFTLTISSLQPDDFATYYCLQYNSWWTFGQGTKVEIK

SEQ ID NO: 342 - protein, 107 aa, Artificial Sequence
  60C4B9-VL6
DIQMTQSPSSLSASVGDRVTITCQASQDIGDWLAWYQQKPGKAPKRLIYGATSLADGVPSRFSGSGSGTEFTLTISSLQPEDFATYYCLQDSDPPYTFGQGTKVEIK

SEQ ID NO: 343 - protein, 107 aa, Artificial Sequence
  98C7H11-VL2
DIQMTQSPSTLSASVGDRVTITCQASQDIGDWLAWYQQKPGKAPKLLIYGATSLADGVPSRFSGSGSGTEFTLTISSLQPDDFATYYCLQDYDPPYTFGQGTKLEIK

SEQ ID NO: 344 - protein, 107 aa, Artificial Sequence
  55D3F10-VL2
DIQMTQSPSSLSASVGDRVTITCQASQDIGDWLAWYQQKPGKAPKLLIYGADTLADGVPSRFSGSGSGTDFTLTISSLQPEDFATYYCLQDSSAPYTFGQGTKVEIK

SEQ ID NO: 345 - protein, 107 aa, Artificial Sequence
  55D3F10-VL6
DIQMTQSPSSLSASVGDRVTITCQASQDIGDWLAWYQQKPGKAPKRLIYGADTLADGVPSRFSGSGSGTEFTLTISSLQPEDFATYYCLQDSSAPYTFGQGTKVEIK

SEQ ID NO: 346 - protein, 12 aa, Artificial Sequence
  73H11G10 VH-CDR3
HGRIFGFGYFDY

SEQ ID NO: 347 - protein, 12 aa, Artificial Sequence
  15B4C6 VH-CDR3
DPLYLLRGAMDY